(19) United States
(12) Patent Application Publication
(10) Pub. No.: US 2007/0154899 A1
(43) Pub. Date: Jul. 5, 2007

(54) Biodetection by nucleic acid-templated chemistry

(76) Inventors: James M. Coull, Westford, MA (US); Andrew M. Stern, Boston, MA (US); Lawrence A. Haff, Westborough, MA (US); Barbara S. Fox, Wayland, MA (US); Yumei Huang, Billerica, MA (US)

Correspondence Address: GOODWIN PROCTER LLP;PATENT ADMINISTRATOR, EXCHANGE PLACE, BOSTON, MA, 02109-2881, US

(21) Appl. No.: 11/441,804

(22) Filed: May 26, 2006

Related U.S. Application Data
(60) Provisional application No. 60/685,047, filed on May 26, 2005.
(60) Provisional application No. 60/701,165, filed on Jul. 21, 2005.
(60) Provisional application No. 60/713,038, filed on Aug. 31, 2005.
(60) Provisional application No. 60/724,743, filed on Oct. 7, 2005.
(60) Provisional application No. 60/758,837, filed on Jan. 13, 2006.
(60) Provisional application No. 60/786,247, filed on Mar. 27, 2006.

(51) Int. Cl. C12Q 1/68; G01N 33/53

(52) U.S. Cl. 435/6; 435/7.1

(57) ABSTRACT

The invention provides compositions and methods for the detection of biological targets, (e.g. nucleic acids and proteins) by nucleic acid templated chemistry, for example, by generating fluorescent, chemiluminescent and/or chromophoric signals.

### RELATED APPLICATIONS

This application claims the benefit of and priority to U.S. Patent Applications Ser. Nos. 60/685,047, filed May 26, 2005; 60/701,165, filed Jul. 21, 2005; 60/713,038, filed Aug. 31, 2005; 60/724,743, filed Oct. 7, 2005; 60/758,837, filed Jan. 13, 2006; and 60/786,247, filed Mar. 27, 2006, the entire disclosure of each of which is incorporated by reference herein for all purposes.

### FIELD OF THE INVENTION

The present invention relates generally to probes and their use in biodetection and diagnostics. More particularly, the invention relates to compositions and methods of nucleic acid templated chemistry (e.g., synthesis of fluorescent, chemiluminescent and chromophoric compounds) in biodetection and diagnostics (e.g., the detection of nucleic acids and proteins).

### BACKGROUND

Fluorescent and colored compounds have been used in the fields of biological research and medicine to detect the presence, absence, state, quantity, and composition of biomolecules. Assays using fluorescent and colored compounds may be performed in vitro, in situ, or in vivo. Examples of commonly used in vitro assays for detection of DNA and RNA are real-time and end-point PCR, DNA sequencing, and DNA microarray technologies.

Nucleic Acid Detection

Common to DNA and RNA detection assays is the requirement for DNA probes and/or primers that bear fluorescent labels. These are typically created by enzymatic and/or chemical synthesis. Other examples of in vitro fluorescent assays include ELISA assays in which an antibody is labeled with a fluorophore. An example of an in situ fluorescent assay is the labeling of whole cells (live or dead) with fluorescently modified antibodies so that they may be detected, imaged, and isolated, for example using a flow sorter. Most recently, there have been efforts to utilize fluorescence as a minimally-invasive detection technology in whole animals. Essentially an antibody or some other bioactive molecule is labeled with a near-IR or IR fluorescent compound and, following injection into the animal; the localization of fluorescence is detected using proper illumination and imaging equipment. In this way cancers and other diseases can be found and monitored without the need for exploratory surgery. The foregoing are just a few examples that illustrate the pervasiveness of fluorescence as a technology for biodetection.

Typically, for most of these types of assays there is a need to remove unbound probe or antibody by a washing step to achieve adequate signal to noise and sensitivity. This adds steps to the assay procedure that result in additional time and cost (reagents and possibly equipment). For DNA/RNA amplification assays such as RT-PCR, washing steps are not required since the target is amplified, effectively reducing the complexity of the sample while providing plenty of analyte for the assay. Yet, even PCR suffers some limitations. For example, the number of analytes that may be detected in a single assay is limited to four or less and the assay requires expensive and power-hungry equipment which limits its applicability to use in the laboratory, and particularly in the field. It would be advantageous to have an assay technology that was as sensitive and specific as PCR, yet was more robust and portable. In the case of in vivo imaging, a “biological” wash step is performed as some period of time is required following injection and before the imaging, to allow the bioactive compound to find its target and to allow excess reagent to clear the body.

Protein Detection

Proteins play a central role in many biological reactions, which are basically composed of intermolecular action and molecular recognition involving various proteins. A common method employed in the identification and quantitative determination of protein uses two-dimensional electrophoresis and mass spectrometry. Another method employs liquid chromatography and mass spectrometry. For the detection of interaction and the identification of proteins, antibody chips have also been used, which are provided with a number of antibodies spotted on the plane surface. Conventional methods using electrophoresis have problems in terms of resolution and detection sensitivity.

U.S. Patent Publication No. 20020064779 by Landegren et al. describes a proximity ligation assay wherein two probes that bind to the target to be detected are enzymatically ligated to the ends of two oligonucleotides that are attached to the two binding probes. The joined oligos are amplified to determine the presence of the target molecule. U.S. Patent Application Publication No. 2005/0009050 by Nadeau et al. describes the similar principle of forming an ampilicon.

U.S. Patent Application Publication No. 20050095627 by Kolman et al. describes a proximity-based assay in which two binding partners linked to two oligonucleotides form a hybrid, partially double stranded DNA structure, upon binding to a target. The partially double stranded structure can then be extended with a DNA polymerase to produce a product which can be further amplified by PCR.

There exists a need for new fluorescent and calorimetric technologies that address many of the shortcomings inherent in the above-mentioned biodetection methods. Many existing detection methods require amplification. There also exists a need for discovery of new fluorescent compounds.

### SUMMARY OF THE INVENTION

The present invention is based, in part, upon the discovery that nucleic acid-templated chemistry can be applied in detection of biological targets, e.g., nucleic acids, proteins, autoantibodies, cells, etc. The present invention is based, in part, upon the discovery that fluorescent, chemiluminescent and chromophoric compounds and reactions generating fluorescent, chemiluminescent and chromophoric signals can be synthesized by nucleic acid-templated chemistry. Such methods, compounds, chemical reactions, and other compositions are useful in detection of biological molecules such as nucleic acids and proteins. Assays of this invention using fluorescent, chemiluminescent and colored compounds may be performed in vitro, in situ, or in vivo.

In one aspect, the present invention relates to a method for detecting a target nucleotide sequence. The method includes (a) providing (1) a first probe comprising (i) a first oligonucleotide sequence and (ii) a first reactive group linked to the first oligonucleotide sequence, and (2) a second probe comprising (i) a second oligonucleotide sequence and (ii) a second reactive group linked to the second oligonucleotide sequence, wherein the first oligonucleotide sequence and the second oligonucleotide sequence are complementary to two separate regions of the target nucleotide; (b) combining the first probe and the second probe with a sample to be tested for the presence of the target nucleotide sequence under conditions where the first probe and the second probe hybridize to their respective complementary regions of the target nucleotide sequence if present in the sample thereby bringing into reactive proximity the first reactive group and the second reactive group; and (c) detecting a reaction between the first reactive group and the second reactive group thereby determining the presence of the target nucleotide sequence.

In another aspect, the invention relates to a method for detecting a target nucleotide sequence. The method includes a) providing a set of probe pairs each probe pair comprising (1) a first probe comprising (i) a first nucleotide sequence and (ii) a first reactive group linked to the first oligonucleotide sequence, and (2) a second probe comprising (i) a second oligonucleotide sequence and (ii) a corresponding second reactive group linked to the second oligonucleotide sequence, wherein the first oligonucleotide sequence and the second oligonucleotide sequence are complementary to two separate regions of the target nucleotide; b) combining the set of probe pairs with a sample to be tested for the presence of the target nucleotide sequence under conditions where each of the first probes and the second probes of the probe pairs hybridizes to its respective complementary region of the target nucleotide sequence if present in the sample thereby bringing into reactive proximity the corresponding pairs of the first and second reactive groups; and c) detecting one or more reactions between the pairs of the first reactive groups and the corresponding second reactive groups thereby determining the presence of the target nucleotide sequence.

In yet another aspect, the invention relates to a method for performing nucleic acid-templated chemistry. The method includes performing multiple nucleic acid-templated chemical reactions that are templated by a single template nucleotide sequence, e.g., under substantially similar conditions and/or substantially simultaneously.

In yet another aspect, the invention provides a method for detecting a biological target. The method includes the following. A first probe is provided. The first probe includes (1) a first binding moiety having binding affinity to the biological target, (2) a first oligonucleotide sequence, and (3) a first reactive group associated with the first oligonucleotide sequence. A second probe is provided which includes (1) a second binding moiety having binding affinity to the biological target, (2) a second oligonucleotide sequence, and (3) a second reactive group associated with the second oligonucleotide sequence. The second oligonucleotide is capable of hybridizing to the first oligonucleotide sequence. The second reactive group is reactive to the first reactive group when brought into reactive proximity of one another. The first and second probes are combined with a sample to be tested for the presence of the biological target under conditions where the first and the second binding moieties bind to the biological target. The second oligonucleotide is allowed to hybridize to the first oligonucleotide sequence to bring into reactive proximity the first and the second reactive groups. A reaction between the first and the second reactive groups is detected thereby determining the presence of the biological target. In one embodiment, the reaction between the first and the second reactive groups produces a fluorescent moiety. In another embodiment, the reaction between the first and the second reactive groups produces a chemiluminescent and/or chromophoric moiety.

In yet another aspect, the invention provides a method for detecting a biological target. The method includes the following. A binding complex is provided of the biological target with a first probe. The first probe includes (1) a first binding moiety having binding affinity to the biological target, (2) a first oligonucleotide sequence, and (3) a first reactive group associated with the first oligonucleotide sequence. The binding complex is contacted with a second probe. The second probe includes (1) a second binding moiety having binding affinity to the biological target, (2) a second oligonucleotide sequence, and (3) a second reactive group associated with the second oligonucleotide sequence. The second oligonucleotide is capable of hybridizing to the first oligonucleotide sequence and the second reactive group is reactive to the first reactive group when brought into reactive proximity of one another. The second oligonucleotide is allowed to hybridize to the first oligonucleotide to bring into reactive proximity the first and the second reactive groups. A reaction is detected between the first and the second reactive groups thereby to determine whether the biological target is present in the sample.

In yet another aspect, the invention provides a method for detecting the presence of a biological target. The method includes the following. A first probe and a second probe are allowed to bind to the target. The first probe includes (1) a first binding moiety having binding affinity to the biological target. (2) a first oligonucleotide sequence, and (3) a first reactive group associated with the first oligonucleotide sequence. The second probe includes (1) a second binding moiety having binding affinity to the biological target, (2) a second oligonucleotide sequence, and (3) a second reactive group associated with the second oligonucleotide sequence. The second oligonucleotide is capable of hybridizing to the first oligonucleotide sequence. The second reactive group is reactive to the first reactive group when brought into reactive proximity of one another. The second oligonucleotide is allowed to hybridize to the first oligonucleotide sequence thereby bringing into reactive proximity the first and the second reactive groups. A reaction between the first and the second reactive groups is detected to determine whether the biological target is present in the sample. In one embodiment, the reaction between the first and the second reactive groups produces a fluorescent moiety. In another embodiment, the reaction between the first and the second reactive groups produces a chemiluminescent and/or cliromophoric moiety.

In yet another aspect, the invention provides a method for detecting the presence of a biological target. The method includes the following. A first probe is provided, which includes (1) a first binding moiety having binding affinity to the biological target, and (2) a first oligonucleotide zip code sequence. A second probe is provided, which includes (1) a second binding moiety having binding affinity to the biological target, and (2) a second oligonucleotide zip code sequence. The first probe is hybridized to a first reporter probe that includes (1) an anti-zip code sequence of oligonucleotides complementary to the first oligonucleotide zip code sequence, (2) a first reporter oligonucleotide, and (3) a first reactive group. The second probe is hybridized to a second reporter probe that includes (1) an anti-zip code sequence of oligonucleotides complementary to the second oligonucleotide zip code sequence, (2) a second reporter oligonucleotide, and (3) a second reactive group. The second reporter oligonucleotide is capable of hybridizing to the first reporter oligonucleotide sequence and the second reactive group is reactive to the first reactive group when brought into reactive proximity of one another. The first and the second probes are contacted with a sample to be tested for the presence of the biological target. The first and the second probes are allowed to bind to the biological target if present in the sample, whereby the second reporter oligonucleotide hybridizes to the first reporter oligonucleotide sequence to bring into reactive proximity the first and the second reactive groups. A reaction between the first and the second reactive groups is detected thereby to determine whether the biological target is present in the sample.

It is worth pointing out the methods of the invention do not require enzymatic or chemical ligation of the first and/or the second oligonucleotide sequences.

In yet another aspect, the invention provides a kit useful for detection of a biological analyte. The kit includes a first probe that includes (1) a first binding moiety having binding affinity to the biological analyte, (2) a first oligonucleotide sequence, and (3) a first reactive group associated with the first oligonucleotide sequence; and a second probe that includes (1) a second binding moiety having binding affinity to the biological analyte, (2) a second oligonucleotide sequence, and (3) a second reactive group associated with the second oligonucleotide sequence. The second oligonucleotide is capable of hybridizing to the first oligonucleotide sequence. The second reactive group is reactive to the first reactive group when brought into reactive proximity of one another.

In yet another aspect, the invention provides a kit useful for detection of a biological analyte. The kit includes a first probe that includes (1) a first binding moiety having binding affinity to the biological target, and (2) a first oligonucleotide zip code sequence; and a second probe that includes (1) a second binding moiety having binding affinity to the biological target, and (2) a second oligonucleotide zip code sequence. The first probe is hybridizable to a first reporter probe comprising (1) an anti-zip code sequence of oligonucleotides complementary to the first oligonucleotide zip code sequence, (2) a first reporter oligonucleotide, and (3) a first reactive group. The second probe is hybridizable to a second reporter probe comprising (1) an anti-zip code sequence of oligonucleotides complementary to the second oligonucleotide zip code sequence, (2) a second reporter oligonucleotide, and (3) a second reactive group. The second reporter oligonucleotide is capable of hybridizing to the first reporter oligonucleotide sequence and the second reactive group is reactive to the first reactive group when brought into reactive proximity of one another.

The invention encompasses a kit that provides one, two or more of the probes described herein. More particularly, the invention encompasses a kit that provides one, two or more of the probes that utilize nucleic acid-templated chemistry for the generation of detectable signals as a way for detecting the presence of a biological target or targets, for example, one or more nucleic acids, one or more proteins, one or more autoantibodies, and/or one or more cells.

The foregoing aspects and embodiments of the invention may be more fully understood by reference to the following figures, detailed description and claims.

### Definitions

The term, “DNA programmed chemistry” or “DPC”, as used herein, refers to nucleic acid-templated chemistry, for example, sequence specific control of chemical reactants to yield specific products accomplished by (1) providing one or more templates, which have associated reactive group(s); (2) contacting one or more transfer groups (reagents) having an anti-codon (e.g., complementary sequence with one or more templates) and reactive group(s) under conditions to allow for hybridization to the templates and (3) reaction of the reactive groups to yield products. For example, in a one-step nucleic acid-templated reaction, hybridization of a “template” and a “complementary” oligonucleotide bring together reactive groups followed by a chemical reaction that results in the desired product. Structures of the reactants and products need not be related to those of the nucleic acids comprising the template and transfer group oligonucleotides. See, e.g., U.S. Patent Application Publication Nos. 2004/0180412 A1 (U.S. Ser. No. 10/643,752; Aug. 19, 2003) by Liu et al. and 2003/0113738 A1 (U.S. Ser. No. 10/101,030; Mar. 19, 2002), by Liu et al.; Gartner, et al., 2004, Science, vol. 305, pp. 1601-1605; Doyon, et al., 2003, JACS, vol. 125, pp. 12372-12373, all of which are expressly incorporated herein by reference in their entireties. See, also, “Turn Over Probes and Use Thereof” by Coull et al., PCT International Patent Application PCT/US06/16999, filed on May 3, 2006.

The terms, “nucleic acid”, “oligonucleotide” (sometimes simply referred to as “oligo”) or “polynucleotide” or as used herein refer to a polymer of nucleotides. The polymer may include, without limitation, natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyluridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). Nucleic acids and oligonucleotides may also include other polymers of bases having a modified backbone, such as a locked nucleic acid (LNA), a peptide nucleic acid (PNA), a threose nucleic acid (TNA).

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, two or more steps or actions may be conducted simultaneously.

### BRIEF DESCRIPTION OF THE FIGURES

The invention may be further understood from the following figures in which:

FIG. 1 is a schematic representation of a method for the detection of nucleic acid targets under one embodiment of the present invention.

FIG. 2 is a schematic representation of an example of detection of low copy number genes via gene painting.

FIG. 3 is a schematic representation of an example of detection of nucleic acid targets by a co-factor release assay.

FIG. 4 is a schematic representation of a method for the detection of a biological target under one embodiment of the present invention.

FIG. 5 is a schematic representation of a method for the detection of a biological target under one embodiment of the present invention.

FIG. 6 shows examples of hybridization as affected by concentration, temperature, and the presence or absence of a single base pair mismatch.

FIG. 7 shows exemplary oligonucleotides used in certain melting curve experiments

FIG. 8 is a schematic representation of a method for the detection of a biological target under one embodiment of the present invention.

FIG. 9 is a schematic representation of a method for the detection of platelet derived growth factor (PDGF) under one embodiment of the present invention.

FIG. 10 shows exemplary embodiment of a splinted, zip-coded detection system with aptamers as target binding moieties.

FIG. 11 shows exemplary embodiment of a splinted, zip-coded detection system with antibodies as target binding moieties.

FIG. 12 is a schematic representation of a method for the detection of a protein target under one embodiment of the present invention.

FIG. 13 shows general structures of polymethine dyes, cyanines and hemicyanines.

FIG. 14 is shows an example of fluorescence signal generation and biological target detection via triphenylphosphine (TPP) and azidocoumarin (AzC) reporter chemistry.

FIG. 15 shows an example of fluorescence signal generation and biological target detection via TPP and AzC reporter chemistry.

FIG. 16 shows certain examples of melt curves illustrating the effect of oligonucleotide concentration on Tₘ.

FIG. 17 shows certain examples with DNA hybridization melting curves of biotinylated oligonucleotides with and without avidin.

FIG. 18 shows certain examples of Tₘ changes of complementary biotinylated oligos upon binding to avidin.

FIG. 19 shows certain examples of the effect of salt and magnesium concentrations upon Tₘ of oligonucleotides+/−biotin.

FIG. 20 shows certain examples of the melting temperature behavior of biotinylated oligonucleotides at different ratios of oligonucleotides to avidin.

FIG. 21 shows certain examples of melting curves of 5′ and 3′ (−) biotin-strand oligos duplexed with biotin-5′ (+) strand oligo in the absence and presence of avidin.

FIG. 22 shows certain examples of melting curves of AT-rich biotinylated oligo dimers with and without avidin.

FIG. 23 is a schematic representation of a method for the detection of a biological target under one embodiment of the present invention.

FIG. 24 shows examples of experimental results on detection of a biological target under one embodiment of the present invention.

FIG. 25A and FIG. 25B show examples of experimental results (the effect of formamide in the reaction mixture) on detection of a biological target under one embodiment of the present invention.

FIG. 26A and FIG. 26B show examples of experimental results (the effect of formamide in the reaction mixture) on detection of a biological target under one embodiment of the present invention.

FIG. 27 shows examples of experimental results (the effect of formamide in the reaction mixture) on detection of a biological target under one embodiment of the present invention.

FIG. 28 shows examples of experimental results (time course of reaction mixtures) on detection of a biological target under one embodiment of the present invention.

FIG. 29 shows examples of experimental results (time course of reaction mixtures) on detection of a biological target under one embodiment of the present invention.

FIG. 30 shows examples of experimental results (probe ratios) on detection of a biological target under one embodiment of the present invention.

FIG. 31 shows an example of detection of PDGF by a zip-coded detection system.

FIG. 32 shows experiments on ratios of aptamers and reporters.

FIG. 33 illustrates an embodiment of a “one-piece” detection system for the detection of PDGF.

FIG. 34 shows exemplary embodiment of a splinted, zip-coded detection system with antibodies as target binding moieties.

FIG. 35 shows a MALDI-MS spectrum of a reaction mixture.

FIG. 36 shows absorption and fluorescence emission spectra of a reaction mixture.

FIG. 37 shows absorption and fluorescence emission spectra of a purified hemicyanine.

FIG. 38 shows an electrospray mass data of a compound.

### DETAILED DESCRIPTION OF THE INVENTION

In its simplest sense, the invention is to generate a detectable signal via a nucleic acid-templated reaction that indicates the presence of a target analyte, e.g., a nucleic acid or a protein, More particularly, the present invention provides an exciting approach to the generation of florescent, chemiluminescent or clromophoric compounds and signals and to utilize such tecnology in biodetection and/or diagnostic applications. Creation and detection of a colored, florescent or chemiluminescent compound or precursor due to the formation or cleavage of a chemical bond, or the chemical transformation of a functional group, directly as the result of a nucleic acid-templated chemical reaction, provide a unique technology that may be applied to many are as including bioterror agent detection and disease diagnostics.

Thus, a hybridization event between probes is followed by a chemical reaction that is mediated by the DNA templates (oligonucleotides), which substantially increases the rate of a chemical reaction due to proximity effect and is able to mediate a variety of chemical reactions, Therefore, the presence of a target biomolecule (e.g., nucleic acid or protein) leads to the onset of a detectable chemical reaction. As a result, the present invention provides easy to use and high signal to noise biological target detection.

Nucleic Acid Detection

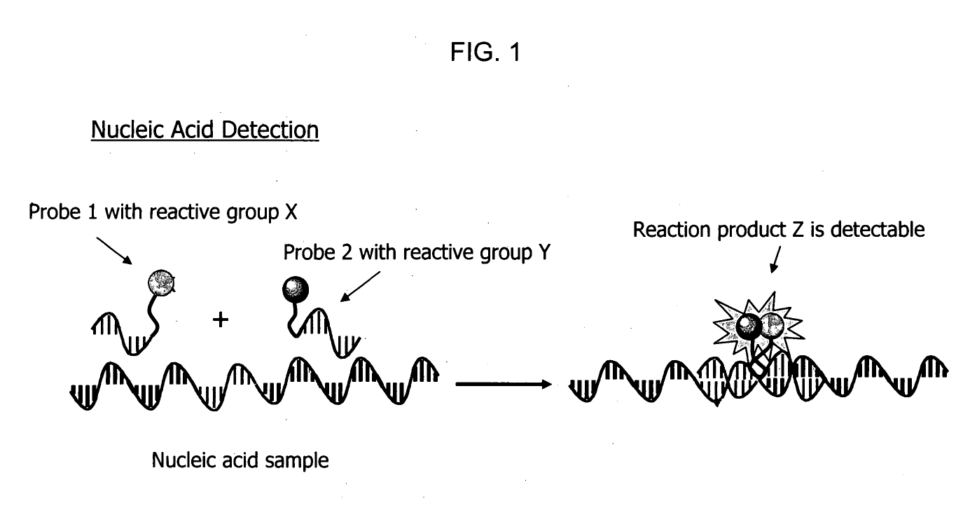

FIG. 1 illustrates an embodiment of detection of a nucleic acid. Two oligonucleotide probes bind to a DNA or RNA target (an analyte, for example, in a sample believed to contain a bioterror or other infectious agents). The two probes are labeled with chemically reactive species X and Y. Upon hybridization, X and Y react to create a signal-generating compound Z (e.g., fluorescent, chemiluminescent or colored compound). Z may or may not covalently link the two probes, and if not, Z may be linked to either probe. Z may be released from the oligonucleotides upon its formation.

If the fluorophore or chromophore is released, it may be separated from the hybridization complex and analyzed independently, or it may be removed once detected so that additional rounds of interrogation of the sample can be conducted (e.g., turnover of probes). If the fluorophore or chromophore is not released, it may also be separated from the rest of the reaction mixture, for example, migrating as a double-stranded structure which can be resolved by gel electrophoresis, for example. The fluorophore attached to the DNA probes on the DNA or RNA target may be attached to a solid-phase such as the surface of a bead, glass slide (microarray), etc. or be in solution, in which case the reaction constitutes a homogeneous assay.

Thus, in one aspect, the present invention relates to a method for detecting a target nucleotide sequence. The method includes (a) providing (1) a first probe comprising (i) a first oligonucleotide sequence and (ii) a first reactive group linked to the first oligonucleotide sequence, and (2) a second probe comprising (i) a second oligonucleotide sequence and (ii) a second reactive group linked to the second oligonucleotide sequence, wherein the first oligonucleotide sequence and the second oligonucleotide sequence are complementary to two separate regions of the target nucleotide; (b) combining the first probe and the second probe with a sample to be tested for the presence of the target nucleotide sequence under conditions where the first probe and the second probe hybridize to their respective complementary regions of the target nucleotide sequence if present in the sample thereby bringing into reactive proximity the first reactive group and the second reactive group; and (c) detecting a reaction between the first reactive group and the second reactive group thereby determining the presence of the target nucleotide sequence.

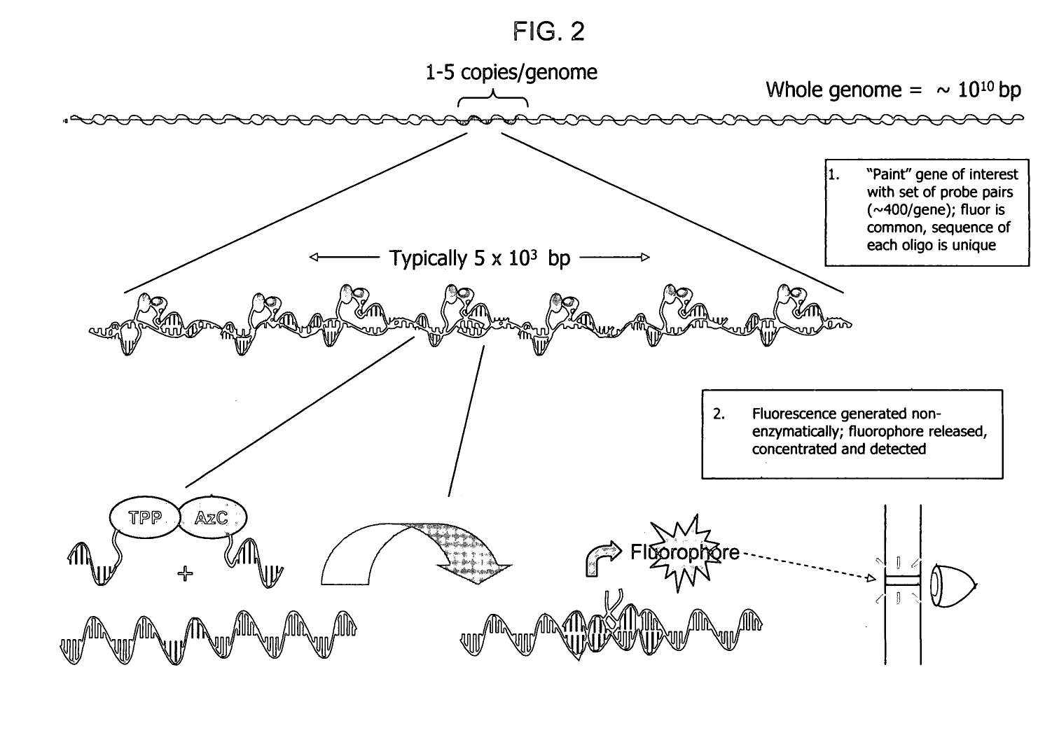

FIG. 2 illustrates an example of detection of a nucleic acid sequence by nucleic acid-templated chemistry enabled detection of low copy number genes. The gene of interest is “painted” with a set of probe pairs (e.g., ˜400/gene). The number of probe pairs can be between, e.g., 2, 5, 10 and 1,000, 5,000 or 10,000. The chemical reactions between the probe pairs (the first reactive groups and the corresponding second reactive groups) may be identical throughout the probe pairs and may be different. Different groups of probe pairs generating different fluorophores can be targeted against different sequences in the target.

The embodiment illustrated in FIG. 2 also may be applied to applications other than biodetection. The principle of multiple nucleic acid-templated reactions occurring on a single DNA template is not limited to generation of fluorescent signal.

Thus, in another aspect, the invention relates to a method for detecting a target nucleotide sequence. The method includes a) providing a set of probe pairs each probe pair comprising (1) a first probe comprising (i) a first nucleotide sequence and (ii) a first reactive group linked to the first oligonucleotide sequence, and (2) a second probe comprising (i) a second oligonucleotide sequence and (ii) a corresponding second reactive group linked to the second oligonucleotide sequence, wherein the first oligonucleotide sequence and the second oligonucleotide sequence are complementary to two separate regions of the target nucleotide; b) combining the set of probe pairs with a sample to be tested for the presence of the target nucleotide sequence under conditions where each of the first probes and the second probes of the probe pairs hybridizes to its respective complementary region of the target nucleotide sequence if present in the sample thereby bringing into reactive proximity the corresponding pairs of the first and second reactive groups; and c) detecting one or more reactions between the pairs of the first reactive groups and the corresponding second reactive groups thereby determining the presence of the target nucleotide sequence.

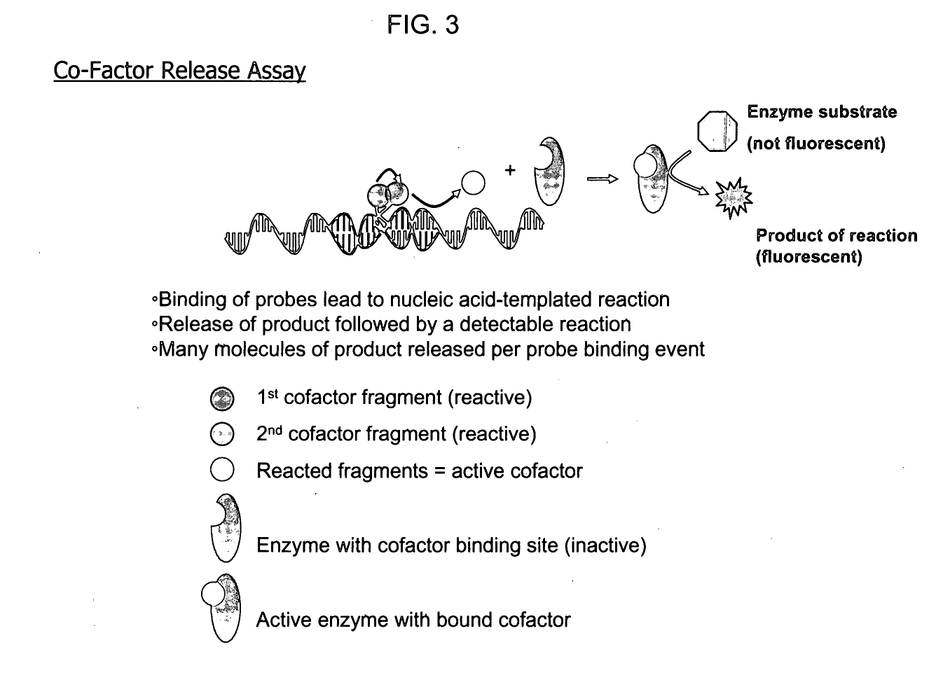

FIG. 3 illustrates an example of another embodiment where an indirect detection scheme involves the nucleic acid-templated reaction followed by a co-factor release and a subsequent detectable reaction.

Protein Detection

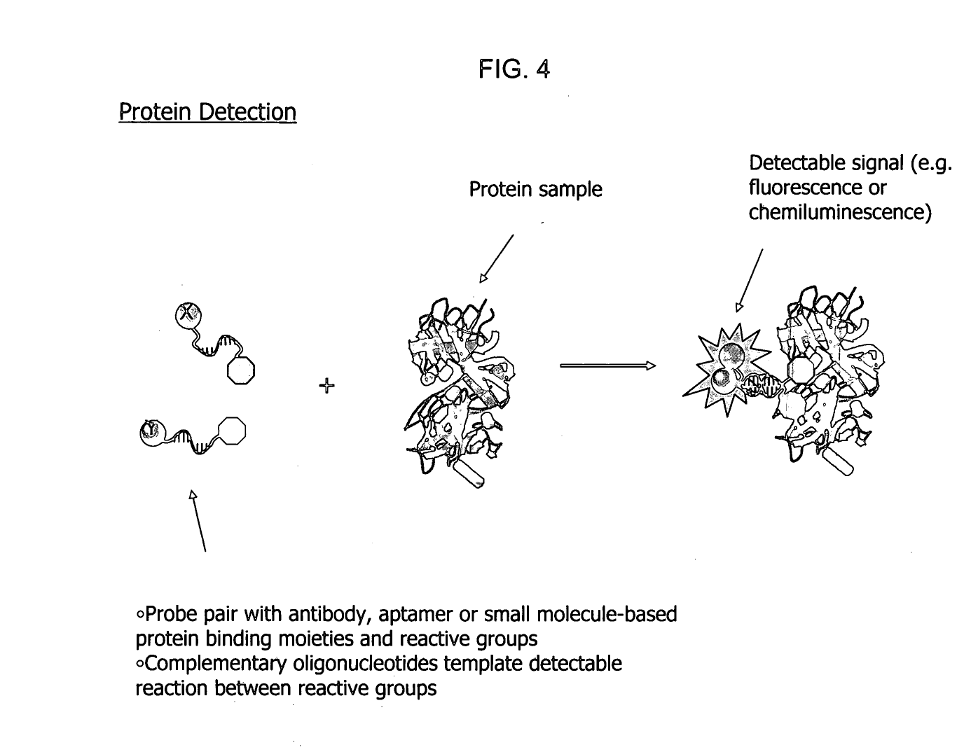

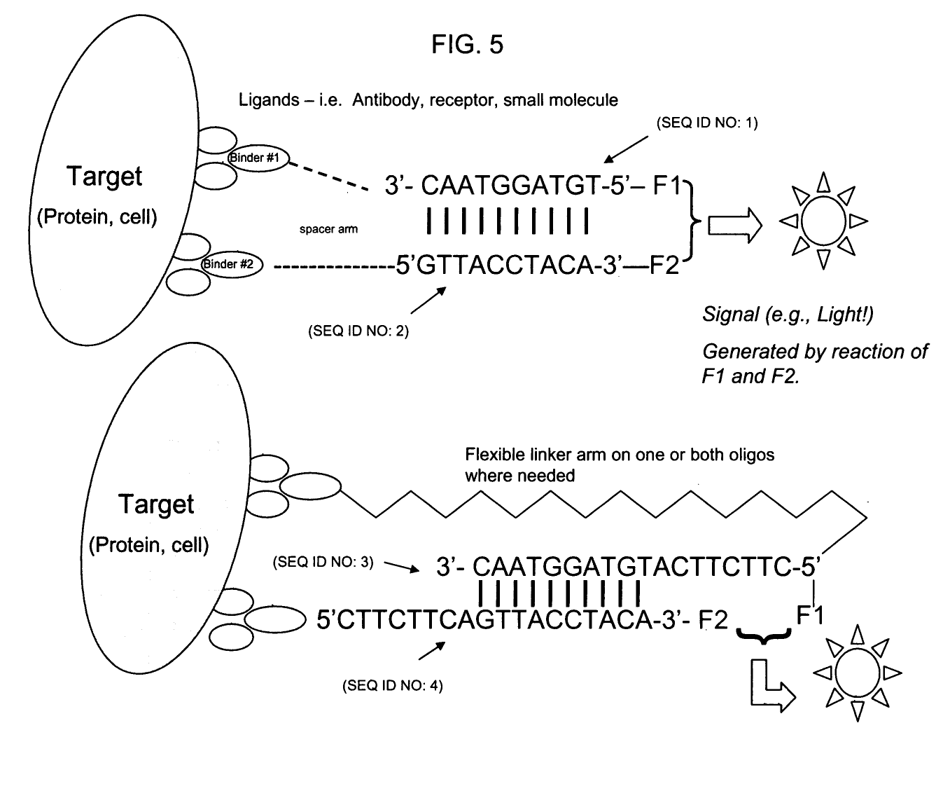

FIG. 4 and FIG. 5 illustrate one embodiment of the invention for the detection of a protein target.

FIG. 4 shows an embodiment of detection of a protein target by the present invention. Two probes contain target binding moieties, complementary oligonucleotides, and chemically reactive species X and Y, respectively. Upon hybridization, X and Y react to create a signal generating (e.g., fluorescent) compound, which may or may not covalently link both probes. The reaction product of X and Y may also be released as an unbound, soluble compound into the solution. The protein target may be attached to a solid-phase such as the surface of a bead, glass slide (microarray), etc., or be in solution. The target binding moieties may be aptamers, antibodies, antibody fragments (i.e., Fab), receptor proteins, or small molecules, for example.

More particularly illustrated in FIG. 5 is an example of the dual-probe approach with two probes, each carrying a “prefluorophore” precursor (F1 and F2) and containing a binding moiety for a target and an oligonucleotide sequence that is designed to anneal to each other. In this embodiment, the detection is performed under conditions such that the prefluorophore oligos will not anneal to each other in the absence of a target. These conditions are generally selected such that the ambient temperature is higher than the Tₘ of the oligonucleotide pairs in the absence of the target (so that the oligo pairs will not anneal in the absence of the intended target analyte). In the presence of the intended target, however, the localized high concentration of the oligos then shifts the Tₘ of their double stranded complex upwards so that hybridization occurs, which is followed by a signal-generating nucleic acid-templated reaction (a reaction between F1 and F2). The signal-generating nucleic acid-templated reaction is accelerated both due to the localized higher concentration of the prefluorophores, but may also be facilitated by the proximity and orientation of the prefluorophore groups towards one another. This configuration of signal generation has the potential to enable creation of kits for the detection of various biomolecules, cells, surfaces and for the design of in situ assays. The signal generation does not require enzymes and the homogeneous format requires no sample manipulation.

In FIG. 5, two oligonucleotides are shown, each of which is linked through an optional spacer arm to a separate binder, as shown in this case is an antibody but may be other binders such as aptamers or small molecules. Each antibody recognizes a separate epitope on a common target analyte such as a protein. Spacer arms can be added to one or both oligonucleotides between the oligo and the binder. In certain cases, this spacer arm may be required to meet proximity requirements to achieve a desired reactivity. Spacer arms in principle can be any suitable groups, for example, linear or branched aliphatic carbon chains C3 to C5, C10, C15, C20, C25, C30, C35, C40, or C100 groups, a DNA sequence of 1 to 10, 15, 20, 30, 50 or 100 bases long, or polyethylene glycol oligomers of the appropriate length.

The prefluorophores may reside in an “end of helix” configuration (FIG. 5 top), one attached to the 5′ end of an oligo and other to the 3′ end. (Other configurations can be applied, including placing the two prefluorophores within the sequence or having one oligo hybridize to a partial hairpin structure (e.g., 100 Angstroms long), for example.) In the first example, one oligonucleotide is attached 5′ to a spacer arm and a target binder, and the other 3′ to a spacer arm and separate target binder. Spacer arms, which can consist of non-complementary DNA sequences, or synthetic spacer arms such as oligomers of ethylene glycol, can be added to meet proximity requirements. Such spacer arms can be very flexible, which has the advantage of overcoming any steric hindrance to binding that might occur with a rigid spacer. A suitably long spacer arm design can permit both oligonucleotides to be linked 5′ to their binders (FIG. 5 bottom), or both linked 3′, as long as the oligonucleotides can anneal in the antiparallel configuration and allow the reactive groups to react with each other. An optimal spacer arm length may be designed for each target. Spacer arms which are excessively long should be avoided as they may reduce specificity in the system or a reduced increased Tₘ effect.

The proximity effect afforded by tethering the pair of oligonucleotides may affect the kinetics of annealing of two complementary oligonucleotide sequences compared to the two oligonucleotides free in solution. More importantly, a localized high concentration shifts the melting curve upwards compared to the free complex, i.e. increase the Tₘ of the complex. In a bulk solution, it is known that Tₘ has dependence upon total oligonucleotide concentration as illustrated in the equation below. Wetmur, Criti. Rev. in Biochem. And Mol. Biol., 26, 227-259 (1991).
Tₘ=(1000*ΔH)/(A+ΔS+R ln(Cₜ/4)−273.15+16.6 log Na⁺)
where ΔH and ΔS are the enthalpy and entropy for helix formation, R is the molar gas constant, C₁ is the total concentration of oligomers, and Na⁺ is the molar concentration of sodium ion in the solution.

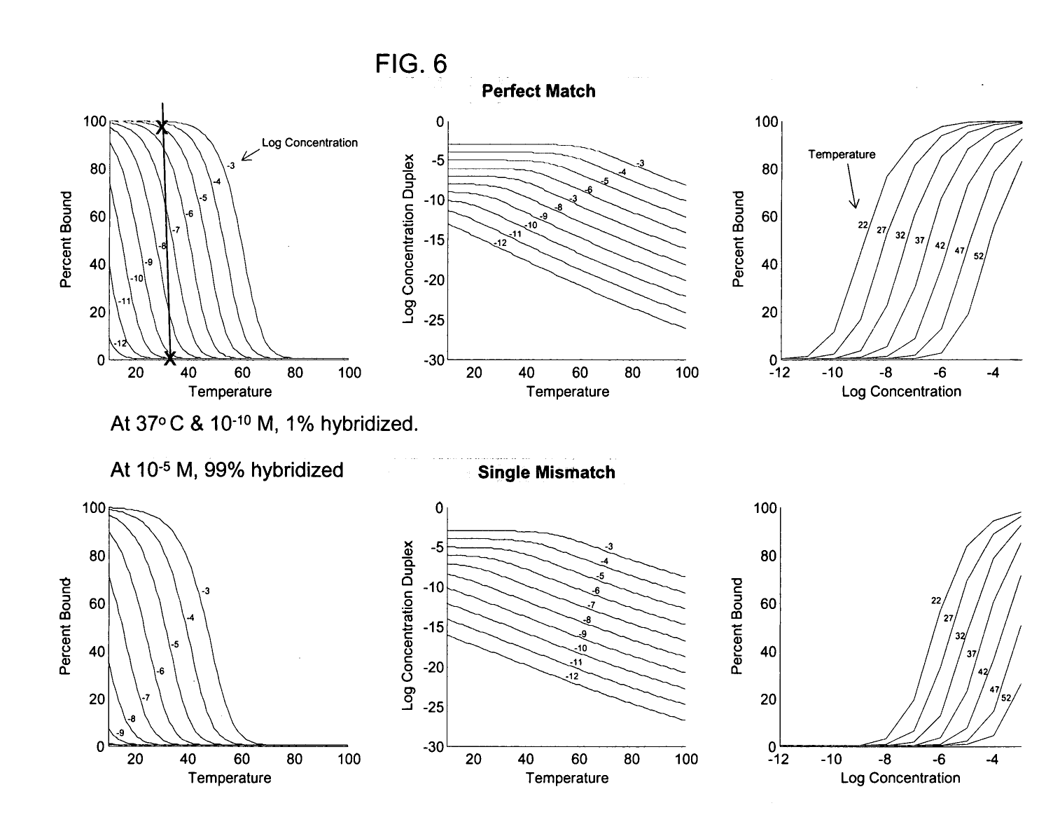

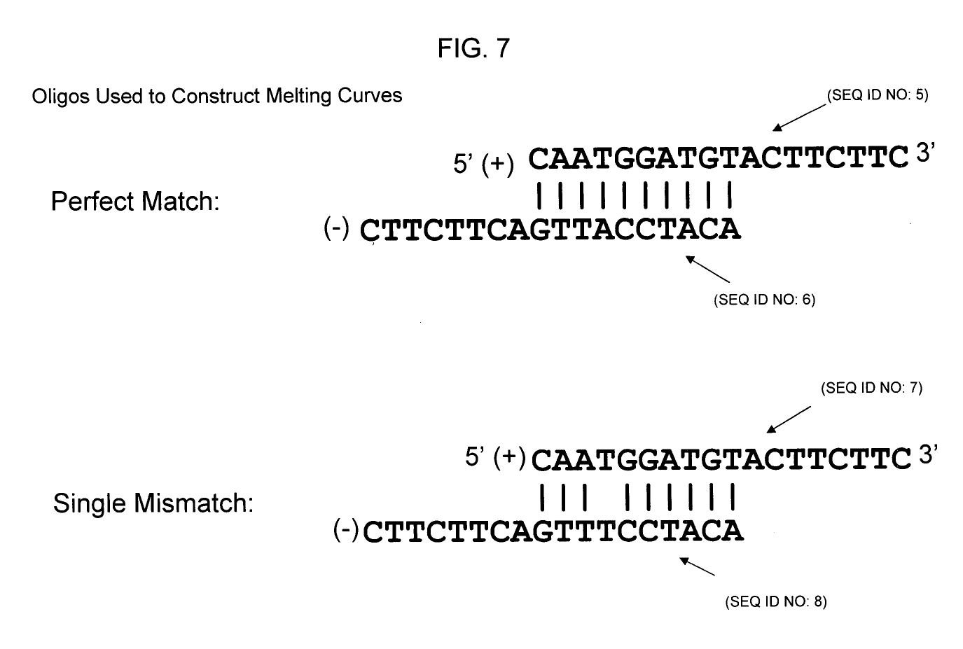

FIG. 6 shows the slope of Tₘ vs. concentration within the range of short oligonucleotides in 0.1 M salt has a dependence of about +7° C. per 10-fold increase in concentration of oligonucleotides (sequences in FIG. 7) based on the above equation. So, for example, a 1000-fold increase in local concentration would be expected to raise Tₘ by about +21° C.

Reaction products of F1 and F2 may be released from the hybridization complex as a result of the chemical transformation. Thus, the fluorophore or chromophore may be separated from the hybridization complex and analyzed independently, or the fluorophore or chromophore and the annealed oligonucleotides may be removed once detected so that additional rounds of interrogation of the sample can be conducted. The reaction between F1 and F2 may or may not covalently link the two probes once the product(s) is formed.

Thus, in one aspect, the invention provides a method for detecting a biological target. The method includes the following. A first probe is provided. The first probe includes (1) a first binding moiety having binding affinity to the biological target, (2) a first oligonucleotide sequence, and (3) a first reactive group associated with the first oligonucleotide sequence. A second probe is provided which includes (1) a second binding moiety having binding affinity to the biological target, (2) a second oligonucleotide sequence, and (3) a second reactive group associated with the second oligonucleotide sequence. The second oligonucleotide is capable of hybridizing to the first oligonucleotide sequence. The second reactive group is reactive to the first reactive group when brought into reactive proximity of one another. The first and second probes are combined with a sample to be tested for the presence of the biological target under conditions where the first and the second binding moieties bind to the biological target. The second oligonucleotide is allowed to hybridize to the first oligonucleotide sequence to bring into reactive proximity the first and the second reactive groups. A reaction between the first and the second reactive groups is detected thereby determining the presence of the biological target. In one embodiment, the reaction between the first and the second reactive groups produces a fluorescent moiety. In another embodiment, the reaction between the first and the second reactive groups produces a chemiluminescent and/or chromophoric moiety.

In another aspect, the invention provides a method for detecting a biological target. The method includes the following. A binding complex is provided of the biological target with a first probe. The first probe includes (1) a first binding moiety having binding affinity to the biological target, (2) a first oligonucleotide sequence, and (3) a first reactive group associated with the first oligonucleotide sequence. The binding complex is contacted with a second probe. The second probe includes (1) a second binding moiety having binding affinity to the biological target, (2) a second oligonucleotide sequence, and (3) a second reactive group associated with the second oligonucleotide sequence. The second oligonucleotide is capable of hybridizing to the first oligonucleotide sequence and the second reactive group is reactive to the first reactive group when brought into reactive proximity of one another. The second oligonucleotide is allowed to hybridize to the first oligonucleotide to bring into reactive proximity the first and the second reactive groups. A reaction is detected between the first and the second reactive groups thereby to determine whether the biological target is present in the sample.

In yet another aspect, the invention provides a method for detecting the presence of a biological target. The method includes the following. A first probe and a second probe are allowed to bind to the target. The first probe includes (1) a first binding moiety having binding affinity to the biological target, (2) a first oligonucleotide sequence, and (3) a first reactive group associated with the first oligonucleotide sequence. The second probe includes (1) a second binding moiety having binding affinity to the biological target, (2) a second oligonucleotide sequence, and (3) a second reactive group associated with the second oligonucleotide sequence. The second oligonucleotide is capable of hybridizing to the first oligonucleotide sequence. The second reactive group is reactive to the first reactive group when brought into reactive proximity of one another. The second oligonucleotide is allowed to hybridize to the first oligonucleotide sequence thereby bringing into reactive proximity the first and the second reactive groups. A reaction between the first and the second reactive groups is detected to determine whether the biological target is present in the sample. In one embodiment, the reaction between the first and the second reactive groups produces a fluorescent moiety. In another embodiment, the reaction between the first and the second reactive groups produces a chemiluminescent and/or chromophoric moiety.

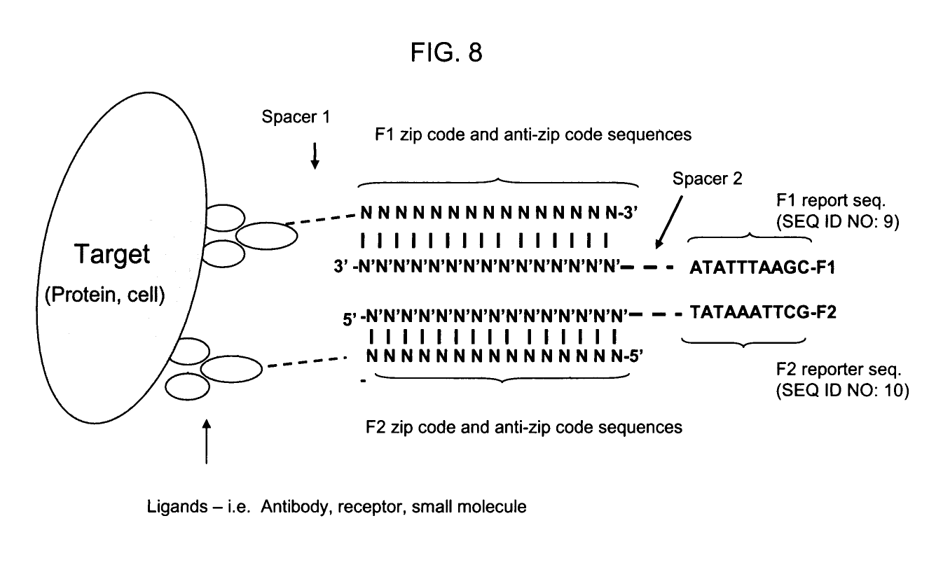

FIG. 8 illustrates another embodiment of the invention, which employs a “zip-coded” splint architecture for nucleic acid template-based biodetection. In this embodiment, instead of the target binding moieties being directly linked (optionally via spacer groups) to the complementary oligonucleotides that hybridize and set up nucleic acid templated reactions, the target binding moieties is linked to a “zip code” oligonucleotide sequence. Each of the corresponding reporter oligonucleotide has a complementary, “anti-zip code” sequence (in addition to a “reporter” sequence that set up nucleic acid-templated reaction). The nucleic acid-templated chemical reactions are set up by the hybridization of the reporter oligos, which are linked to reactive groups that react and generate detectable signals. It is important that each oligonucleotide sequence of the probes is complementary only to its intended hybridization partner and not complementary to other oligonucleotides in the detection system.

This zip-coded architecture supports creating a single reporter-oligonucleotide conjugate which would assemble with different downstream reporter oligonucleotides through an anti-zip code sequence. Libraries of different reporters linked to a unique anti-zip code may be tested simply by mixing each one with stoicheometric amounts of the binder-zip code oligonucleotide conjugate with its complementary zip code.

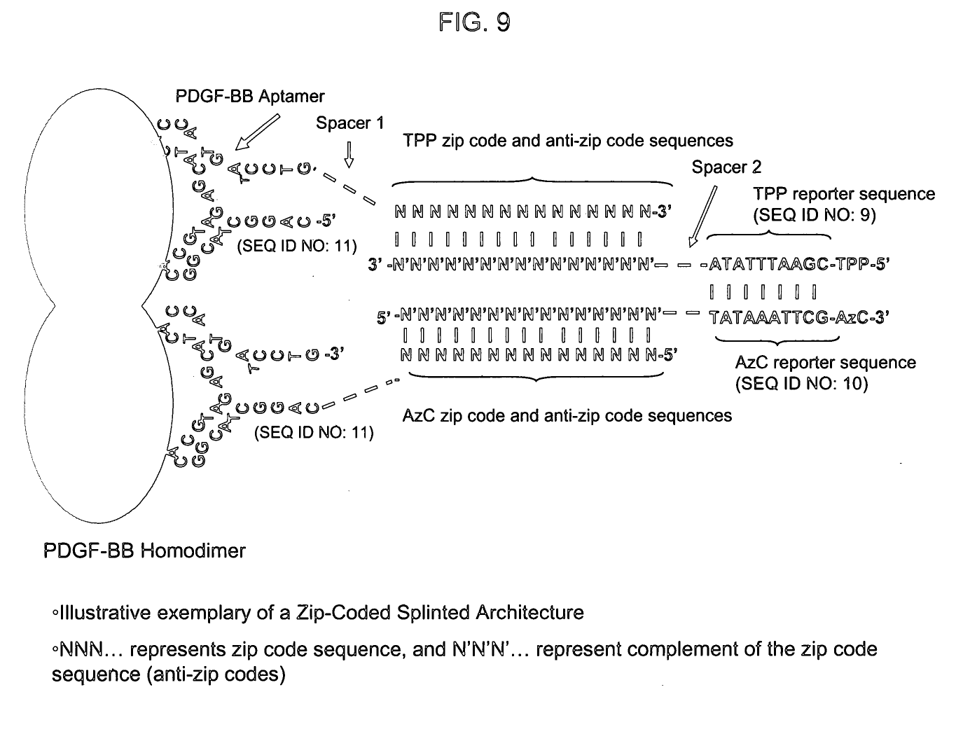

FIG. 9 is an illustration of a zip-coded splinted architecture approach where the target binding moieties are two aptamers. In this example for detection of platelet derived growth factor (PDGF) with illustrative oligo sequences and reporter chemistry (e.g., triphenylphosphine, TPP, and 7-azidocoumarin, AzC), the TPP reporter oligonucleotide self-assembles to the PDGF aptamer oligonucleotide through hybridization of zip code sequence (NNN . . . ) to the complementary anti zip code sequence (N′N′N′ . . . ) on the TPP reporter oligonucleotide. The reporter oligonucleotide terminates with an exemplary 10-base reporter sequence and a 5′-TPP group. A separate pair of oligonucleotides, with different zip codes and anti-zip codes (complementary to each other pairwise), also self-assembles to provide the AzC reporter sequence and a 3′-AzC group. The AzC oligonucleotides are complementary and antiparallel to the TPP oligonucleotides so the TPP and AzC groups terminate end-to-end when the TPP and AzC oligonucleotides anneal to each other.

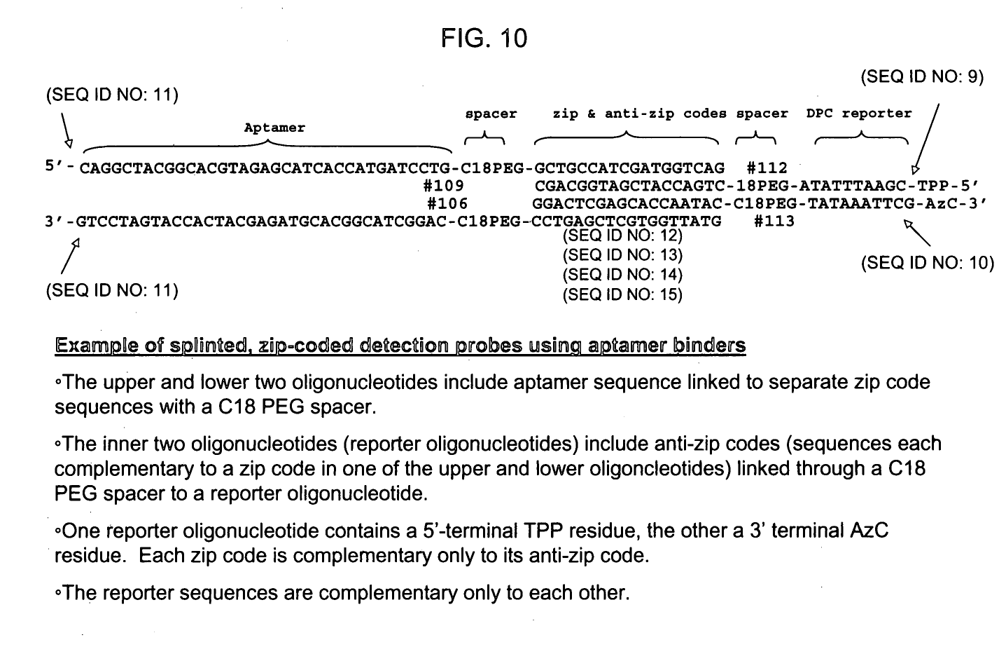

FIG. 10 illustrates in more detail the zip-coded splinted architecture approach for detection of PDGF with illustrative oligo sequences and reporter chemistry (TPP and AzC). The TPP pair includes, first, a PDGF-aptamer on the 5′-end, a C18 polyethylene-glycol based spacer, and an 18-mer zip code sequence. The TPP reporter sequence includes a complementary anti-zip code sequence on its 3′ terminus, a C18 PEG spacer, and a ten base pair reporter sequence terminating in a 5′ TPP group. The AzC pair of oligonucleotides includes a 3′-aptamer linked through a C18 PEG spacer to a separate zip code, and a detection oligonucleotide linked to a 5′ anti-zip code, a C18 PEG spacer, and a reporter oligonucleotide (complementary to the TPP oligonucleotide) terminating in a 3′ AzC group.

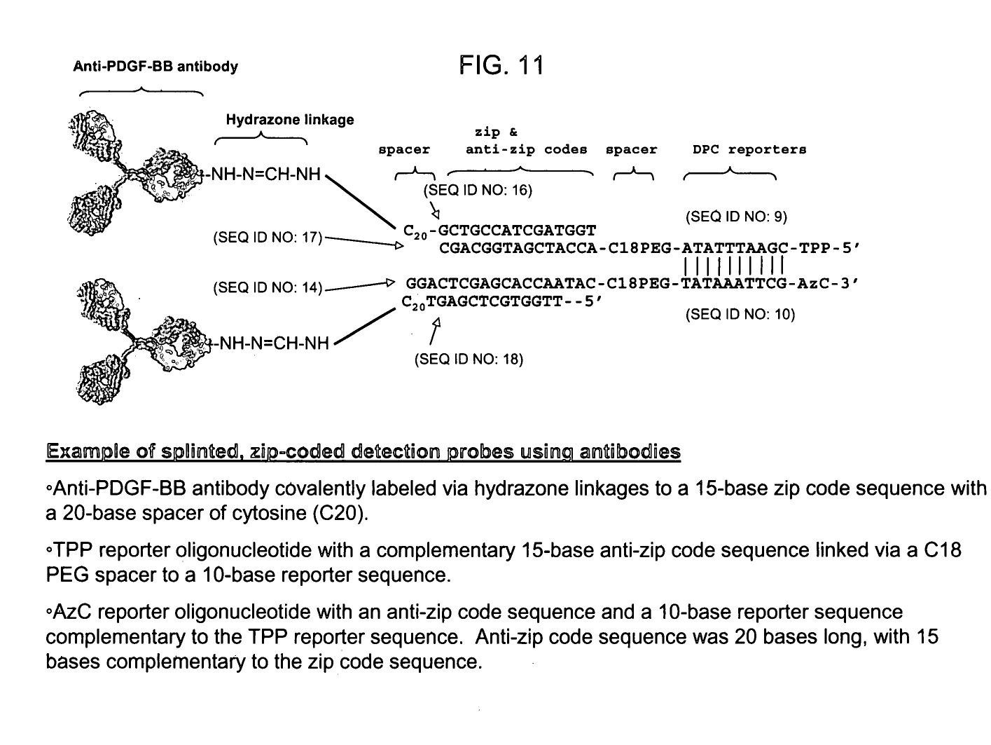

FIG. 11 illustrates an example of the corresponding architect where antibodies are used instead of aptamers as target binding moieties.

One advantage of the “zip coded” approach is the ability to create the reporter oligonucleotides separately, and have them assemble together with binders under conditions retaining the activities of both the binders and of the nucleic acid template-activated chemistry.

The zip-coded system is based upon two pairs of oligonucleotides, with each pair being held together by the base-pairing of a unique zip code and an anti-zip code pair. “Zip codes” are oligonucleotide sequences which bind specifically to their complementary sequences, and preferably are designed such they are not complementary to known genomic sequences (relevant if the sample may contain genomic DNA), have similar Tₘ values, lack significant secondary structure, and do not anneal to other zip code or anti-zip code sequences in the detection system.

Thus, another aspect of the invention provides a method for detecting the presence of a biological target. The method includes the following. A first probe is provided, which includes (1) a first binding moiety having binding affinity to the biological target, and (2) a first oligonucleotide zip code sequence. A second probe is provided, which includes (1) a second binding moiety having binding affinity to the biological target, and (2) a second oligonucleotide zip code sequence. The first probe is hybridized to a first reporter probe that includes (1) an anti-zip code sequence of oligonucleotides complementary to the first oligonucleotide zip code sequence, (2) a first reporter oligonucleotide, and (3) a first reactive group. The second probe is hybridized to a second reporter probe that includes (1) an anti-zip code sequence of oligonucleotides complementary to the second oligonucleotide zip code sequence, (2) a second reporter oligonucleotide, and (3) a second reactive group. The second reporter oligonucleotide is capable of hybridizing to the first reporter oligonucleotide sequence and the second reactive group is reactive to the first reactive group when brought into reactive proximity of one another. The first and the second probes are contacted with a sample to be tested for the presence of the biological target. The first and the second probes are allowed to bind to the biological target if present in the sample, whereby the second reporter oligonucleotide hybridizes to the first reporter oligonucleotide sequence to bring into reactive proximity the first and the second reactive groups. A reaction between the first and the second reactive groups is detected thereby to determine whether the biological target is present in the sample.

It is worth pointing out the methods of the invention do not require enzymatic or chemical ligation of the first and/or the second oligonucleotide sequences.

Factors that may be considered in optimizing a design of a zip-coded architecture include, for example, (1) spacer groups (e.g., oligonucleotides and/or non-base groups) between the aptamer/antibody and zip codes (spacer 1), e.g., to allow hybridization partners to reach each other, to prevent any steric hindrance; (2) Length of a zip code sequence in order to form a sufficiently stable annealing to the anti-zip code sequence to form the complex; and (3) Spacer groups (spacer 2) between the anti-zip code and the reporter sequence, e.g., to prevent any steric hindrance.

The binders (target binding moieties) attached to the oligonucleotides may be any chemical moieties that specifically bind to a target molecule and allow the design of the invention to work. Examples include a wide range of functionalities, such as (1) antibodies: e.g., IgG, IgM, IgA, IgE, Fab's, Fab', F(ab)₂, Dab, Fv or ScFv fragments; (2) small molecule binders, such as inhibitors, drugs, cofactors; (3) receptors for protein detection, and vice versa; (4) DNA, RNA, PNA aptamers; (5) DNA sequences for DNA-binding and regulatory proteins; (6) peptides representing protein binding motifs; (7) peptides discovered through phage display, random synthesis, mutagenesis; (8) naturally binding protein pairs and complexes; (9) antigens (for antibody detection); and (10) a single polyclonal antibody separately attached to two oligonucleotides may serve as two separate binders of different specificity.

The target binding moieties attached to the oligonucleotides may be of heterogeneous types directed against different sites within the same target. For example, the two binders may be two different antibodies, an antibody and a receptor, an antibody and a small molecule binder, a receptor and a peptide, an aptamer and a cofactor, or any other combination.

The target analytes can be of any type, provided the target supports two (or more) binding sites. The two binding sites may be identical or not identical. In the case of identical sites, the benefits of increased specificity obtained with two non-identical binders will not be obtained. Molecules which exist in equilibrium with a monomeric form and a homodimeric or higher polymerization phase may be detected by a pair of probes containing the same binder but different complementary DNA sequences. Suitable targets include proteins, cell surfaces, antibodies, antigens, viruses, bacteria, organic surfaces, membranes, organelles, in situ analysis of fixed cells, protein complexes. The invention may be particularly suited for the detection of fusion proteins (e.g., BCR-ABL in the presence of BCR and ABL).

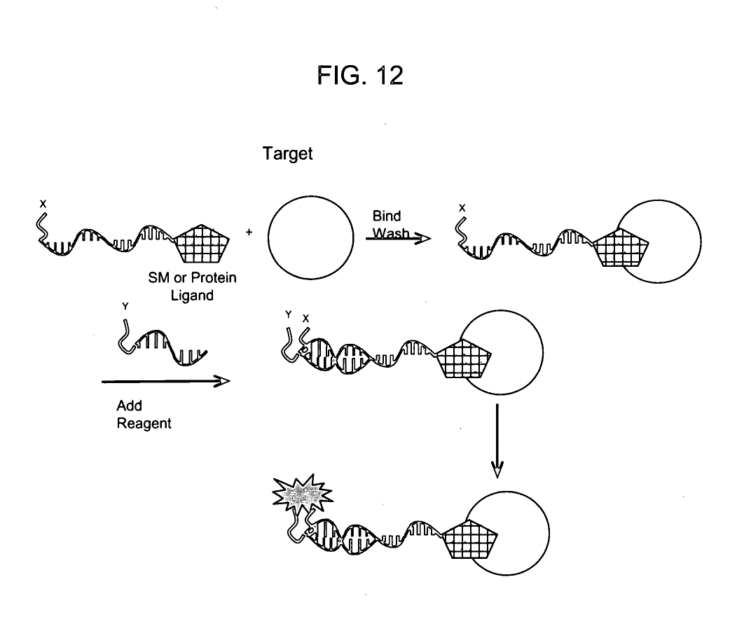

FIG. 12 shows an embodiment of how a protein or small molecule binding assay may be reported using the synthesis of a fluorophore or chromophore via nucleic acid-templated chemistry. In this example protein binders such as an aptamers, an antibody, or a small molecule binder, represented by a pentagon is conjugated to an oligonucleotide (a “template”) having a reactive group X on its terminus. The sample is mixed with binder-template and if the analyte of interest is present (represented by a circle) a complex is formed. Excess binder-template is removed, and a probe bearing a reactive group Y and an oligonucleotide complementary to the above template is added. Hybridization of the oligonucleotides sets up a reaction between X and Y, creating a detectable signal molecule (e.g., a fluorophore or chromophore).

The signal molecule (represented by a star) may remain attached to the probe-template hybrid, or may be released from the complex. The analyte may be attached to a solid-phase or may be free in solution so long as excess binder-template is removed before addition of the probe bearing Y.

Because the template and the probe uniquely encode the synthesis of the reporter, and many different reporters can be envisioned, a multiplex system may be designed. For example, a range of fluorophores with spaced (e.g. evenly spaced) emission may be created, allowing two, three, four, five or more analytes to be detected simultaneously. Moreover, a system may be designed in which both colored and fluorescent compounds are created simultaneously.

In the design of the probes, one consideration is the Tₘ of the two reporter sequences carrying the reactive groups. Since the Tₘ of the duplex should be below room temperature in the absence of a target, this sequence normally should be short, for example 6-15 bases and/or A-T rich. A typical reporter length of 10 base pairs might have a Tₘ of around 30° C. at a low salt concentration. Therefore, it is often necessary even with a short sequence to add 10% to 40% volume/volume formamide to further lower the temperature below assay temperature, or to elevate the assay temperature. Very short reporter oligonucleotides may suffer from a lack of specificity and exhibit some binding to zip code sequences (when these are employed) which is undesirable.

Another factor in the design of the probes is the length of oligonucleotide in between the binding moiety and the reporter sequence, including any zip code sequences. These must be long enough for the reporter oligonucleotides to reach each other and anneal. The sequences may, be interspersed with polyethylene glycol (PEG) linkers that are flexible and may afford additional protection against any steric hindrance. For example, total lengths of oligonucleotides may be around 35 bases long. Oligonucleotides containing 0, 1, or 2 C18 PEG spacers, or homopolymer tracts may also be utilized (i.e. C₁₀).

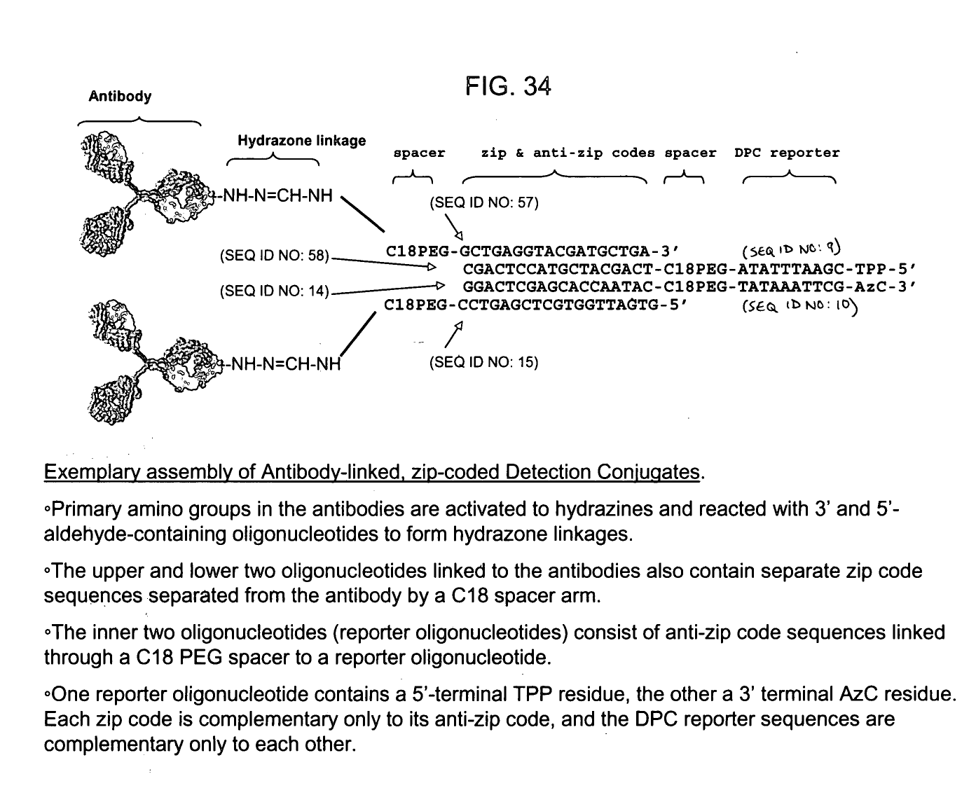

A third consideration is the length of zip and anti-zip sequences when these are employed (i.e. FIG. 9 and FIG. 34). Aside from the need for each zip code to anneal only to its anti-zip code, and not any other zip code, anti-zip code, or reporter sequence, an important parameter is the Tₘ of the duplex between the zip codes and anti-zip codes. The Tₘ should be substantially higher than the highest temperature that will be used in the assay in order that the reporter oligonucleotides remain firmly attached to the binding moiety. In practice, zip codes of about twice the length of the reporter sequences (i.e. total length of 15-30 bases) are desirable and generally meet these criteria.

Regarding signal generation, nucleic acid-templated chemistry may be used to create or destroy a label that effects an optical signal, e.g., creating or destroying a fluorescent, chemiluminescent, or calorimetric molecule. Additionally, a detection reaction may be designed to create or destroy a product that directly or indirectly creates a detectable label, for example, a product that catalyzes a reaction that creates an optical label; inhibits a reaction that creates an optical label; is a fluorescence quencher; is a fluorescent energy transfer molecule; creates a Ramen label; creates an electrochemiluminescent label (i.e. ruthernium bipyridyl); produces an electron spin label molecule.

Furthermore, a detection reaction may be designed to involve a “label-less” detection. Nucleic acid templated chemistry can be used to create or destroy a molecule discernable by an inherent native property of the molecule, for example, a product that creates light-scattering label or aggregation; is detectable by microcalorimetry; is detectable (e.g. an epitope) by surface plasmon resonance (i.e. binding to an immobilized antibody); creation or destruction of an epitope recognized by an antibody (i.e. ELISA); with discernable mass, measured by mass spectrometry; of altered size, discernable by light scattering, gel electrophoresis or size exclusion chromatography; of altered hydrophobicity or ionic content discerned by chromatography; of altered affinity to an affinity chromatography separation.

Another aspect of the invention provides a kit useful for detection of a biological analyte. The kit includes a first probe that includes (1) a first binding moiety having binding affinity to the biological analyte, (2) a first oligonucleotide sequence, and (3) a first reactive group associated with the first oligonucleotide sequence; and a second probe that includes (1) a second binding moiety having binding affinity to the biological analyte, (2) a second oligonucleotide sequence, and (3) a second reactive group associated with the second oligonucleotide sequence. The second oligonucleotide is capable of hybridizing to the first oligonucleotide sequence. The second reactive group is reactive to the first reactive group when brought into reactive proximity of one another.

In yet another aspect, the invention provides a kit useful for detection of a biological analyte. The kit includes a first probe that includes (1) a first binding moiety having binding affinity to the biological target, and (2) a first oligonucleotide zip code sequence; and a second probe that includes (1) a second binding moiety having binding affinity to the biological target, and (2) a second oligonucleotide zip code sequence. The first probe is hybridizable to a first reporter probe comprising (1) an anti-zip code sequence of oligonucleotides complementary to the first oligonucleotide zip code sequence, (2) a first reporter oligonucleotide, and (3) a first reactive group. The second probe is hybridizable to a second reporter probe comprising (1) an anti-zip code sequence of oligonucleotides complementary to the second oligonucleotide zip code sequence, (2) a second reporter oligonucleotide, and (3) a second reactive group. The second reporter oligonucleotide is capable of hybridizing to the first reporter oligonucleotide sequence and the second reactive group is reactive to the first reactive group when brought into reactive proximity of one another.

The invention encompasses a kit that provides one, two or more of the probes described herein. More particularly, the invention encompasses a kit that provides one, two or more of the probes that utilize nucleic acid-templated chemistry for the generation of detectable signals as a way for detecting the presence of a biological target (e.g., nucleic acid and proteins).

### Reporter Chemistries

Coumarins

Coumarins may be used in reporter chemistry, particularly coumarins bearing electron donating substituents at the 7-position. The scheme below illustrates how the reduction of a 7-azidocoumarin (known to be non-fluorescent) to the 7-aminoderivative (fluorescent) can be accomplished using nucleic acid-templated chemistry. 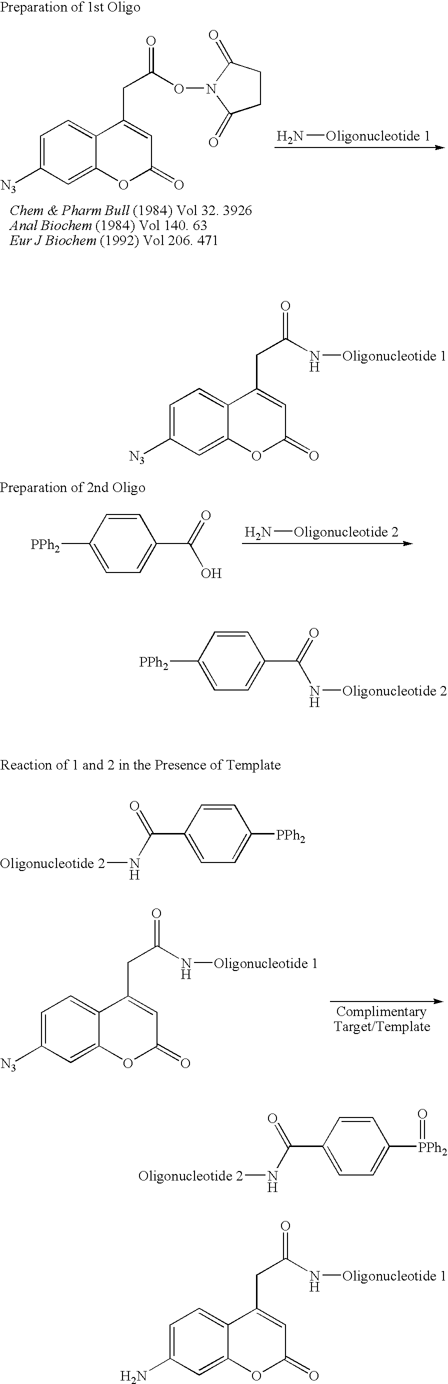
Fluorescamine

Following on with the use of phosphines to reduce azides to amines, one can react the resulting amine with a free (not attached to DNA) reagent to form a fluorescent amine derivative. A prime example is fluorescamine which is intrinsically non-fluorescent but produces a blue-green fluorescent product upon reaction with a primary or secondary amine. 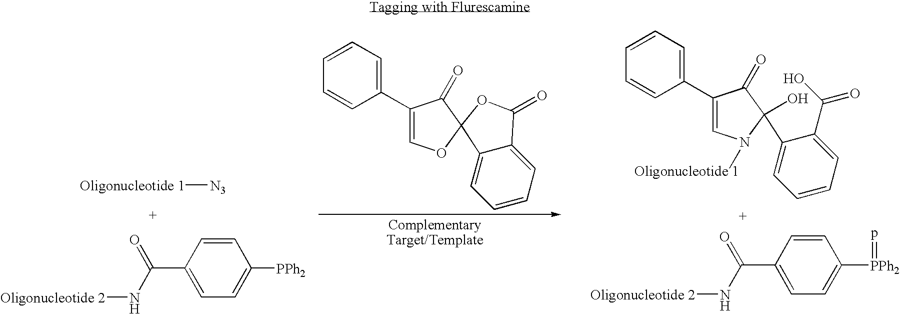
Isoindole Derivatives

The reaction or trapping of two functional groups that are in close proximity with a derivatizing reagent may also be utilized. These two functional groups may be on two different oligos and be brought together by the hybridization event, or they may both be on a first oligo whereby a second oligo is used to unmask or transform one or more of the groups into a species that can be derivatized. This is illustrated below for the formation of isoindoles from o-dialdehydes and ketones which are commonly used as amine detection reagents. The detection limit for 3-(4-carboxybenzoyl)quinoline-2-carboxaldehyde (CBQCA)-derivatized amines is reported to be in the attomole range. 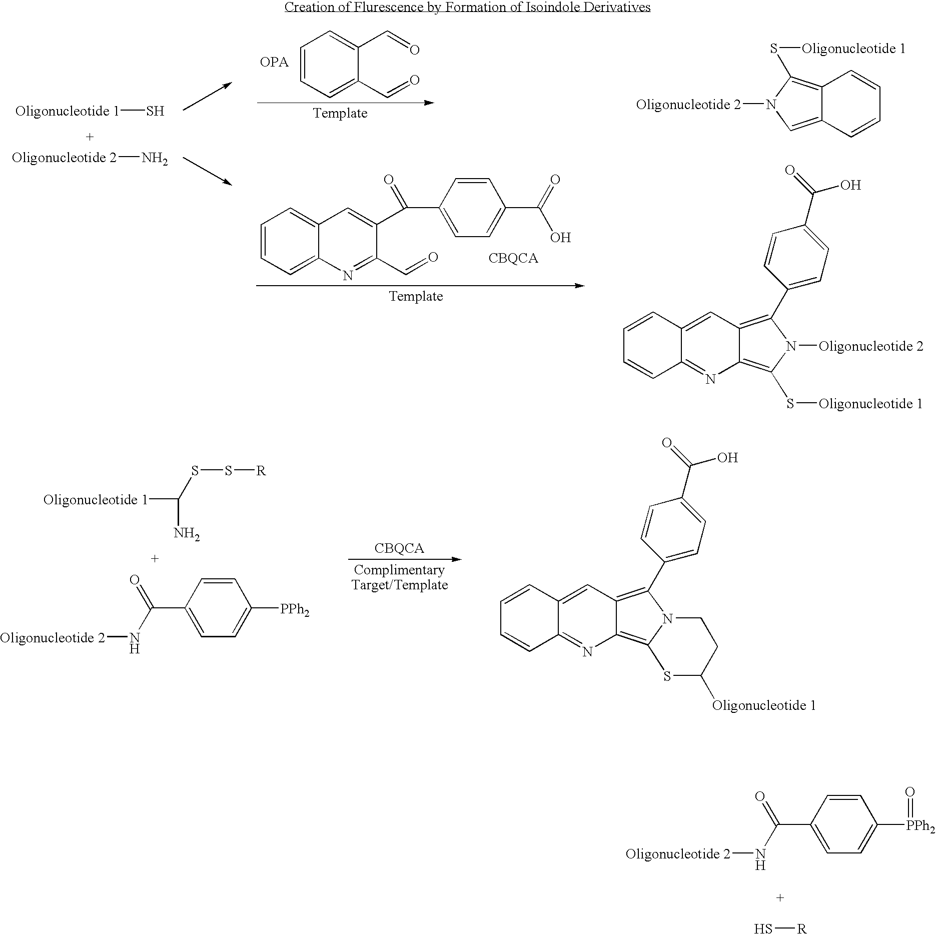
Polymethine Dye Reporter Chemistry

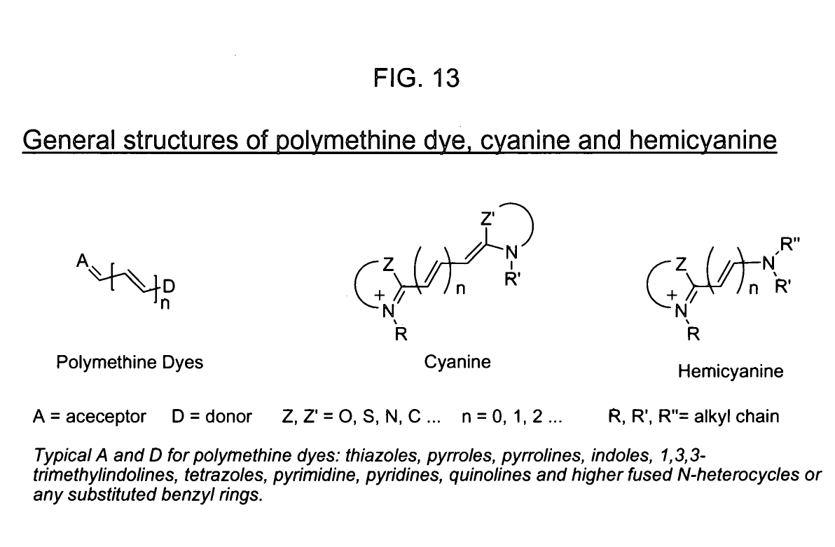

Polymethine dye is characterized by a chain of methine (—CH═) groups with an electron donor and an electron acceptor at opposite ends of their polyene chain (FIG. 13, Zollinger, Color Chemistry: Syntheses, Properties, and Applications of Organic Dyes and Pigments, 3nd Edn., Verlag Helvetica Chimica Acta, Postfach, Switzerland, 2003). Typical A and D terminals for polymethine dyes (as shown in FIG. 13) include thiazoles, pyrroles, pyrrolines, indoles, 1,3,3-trimethylindolines, tetrazoles, pyrimidine, pyridines, quinolines, and higher fused N-heterocycles or any substituted benzyl rings. If the terminals are both N-atom containing heterocycles, the compound is named cyanine. If only one N-atom is part of the ring system, the compound is named hemicyanine. By changing the number of the vinyl group in the polyene chain, the fluorescence emission wavelength of the polymethine dye can be tuned from near-UV to near-IR. The terminal group may also provide mean for finer tuning.

Polymethine dyes are generally synthesized by nucleophilic and/or electrophilic substitutions, preceded or followed by deprotonation (Raue, Ullmann's Encyclopedia of Industrial Chemistry, 5^{th} Edn., UCH, Weinheim 1990, Vol. A16, p487.) Scheme 1 below is an example of an asymmetric cyanine dye synthesis. 2-Methyl heterocyclic quaternary salt reacts with one equivalent of electrophilic coupling reagent diphenylformamidine to form amidine or hemicyanine. Stepwise nucleophilic addition of second heterocyclic quaternary salt leads to asymmetrical cyanine dye. N-acylated hemicyanine may react with second heterocycle on solid phase under relatively mild condition (Mason, et al., J. Org. Chem. 2005, 70, 2939-2949).

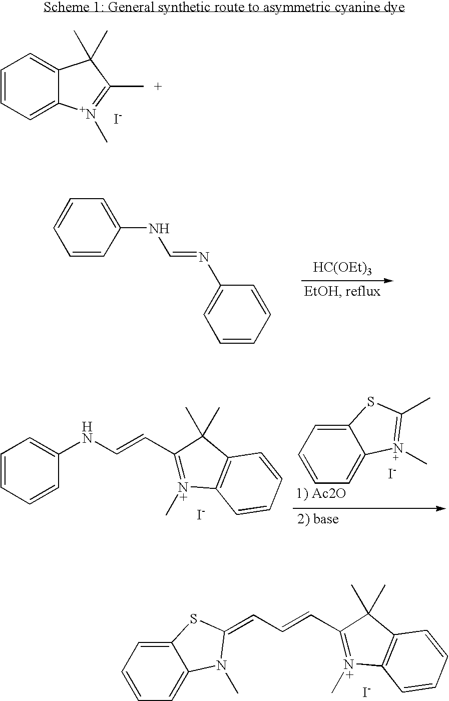

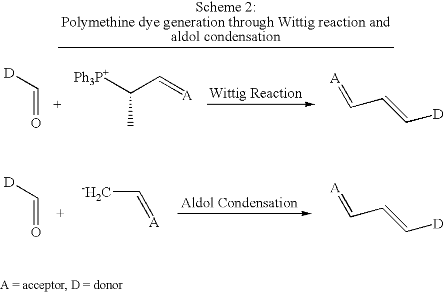

Aldol condensation has been frequently used to synthesize hemicyanine dyes (Hassner, et al., J. Org. Chem. 1984, 49, 2546-2551; Jedrzejewska, et al., Dyes and Pigments 2003, 58, 47-58; Sczepan, et al., Photochem. Photobiol. Sci. 2003, 2, 1264-1271). Here the active-hydrogen component is a quaternary salt while the carbonyl component has an amino-substituent on the aromatic ring. This type of aldol condensation is generally performed under reflux condition in anhydrous alcohol with catalytic amount of base, however, aqueous condition has also been attempted for some active aldehydes (potassium carbonate dilute solution, pH 8, 70° C., 24 hr; reference: Wang, et al., Dyes and Pigments 2003, 59, 163-172).

By choosing aldehyde and the quaternary salt bearing active-hydrogen with optimized chemical activities, aldol condensation may be used for the synthesis of polymethine dye under nucleic acid-templated reaction conditions. DNA-conjugated aldehyde and quaternary salt bearing active-hydrogen may be utilized in detection systems of the present invention. The general approach described here can also be used to attach these precursors to other biopolymers such as sugars, peptides and proteins. The general method for synthesis of polymethine dye by aldol condensation under aqueous condition and the generation of polymethine dye through nucleic acid-templated reaction are useful reporter chemistries.

Wittig reaction allows the preparation of an alkene by the reaction of an aldehyde or ketone with the ylide generated from a phosphonium salt. So far, there is little literature on the synthesis of hemicyanine through Wittig reaction (Zhmurova, et al., Zhurnal Organicheskoi Khimii, 1975, 11, 2160-2162.). Here, the aldehyde and ylide were refluxed in sodium phenolate containing benzene for 9 hr.

While Wittig reagent is known to be able to react with aldehyde at mild basic condition via nucleic acid-templated chemistry (Gartner, et al., J. Am. Chem. Soc. 2002, 124, 10304-10306), the general strategy of synthesis of polymethine dye by nucleic acid-templated Wittig reaction as well as methodologies for synthesizing the Wittig reagent precursors described here are useful reporter chemistries.

(i) Synthesis of Polymethine Dye by Wittig Reaction in Aqueous Solution

Switching the Wittig reaction condition from anhydrous to aqueous media, fast reaction and high yield can be achieved for the synthesis of polymethine dyes. Schemes 3 and 4 below provide two separate examples for the synthesis of cyanines and hemicyanines under aqueous condition.

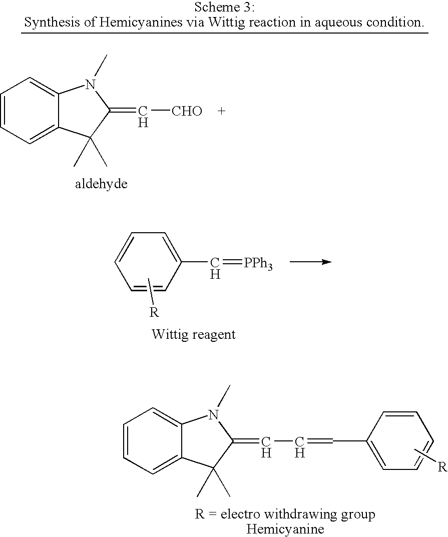

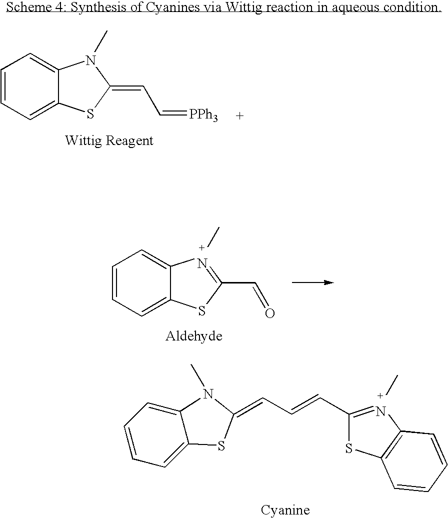

(ii) Attachment of Precursors to DNA

The precursor for aldol and Wittig reactions can be easily conjugated to DNA through amide bond formation. First, an acid heterocyclic or aromatic precursor is synthesized. The acid is then converted to the active N-hydroxysucciimide ester that readily reacts with DNA bearing amine functionality.

(iii) Synthesis of Aldehyde Precursors for Aldol Condensation and Wittig Reaction

The acid functionality in aldehyde precursors is introduced either through quaternization if a nitrogen containing heterocycle is involved (Scheme 5 and Scheme 6) or hydrolysis of a cyano group by hydrogen peroxide if a cyano substituted aromatic aldehyde is involved, for example. Disilylated tert-butylacetaldimine or Wittig reagents can be used repeatedly for the two-carbon homologation of aldehydes into the corresponding α,β-enals if the extensively conjugated aldehyde is required (Bellassoued, et al., J. Org. Chem. 1993, 58, 2517-2522).

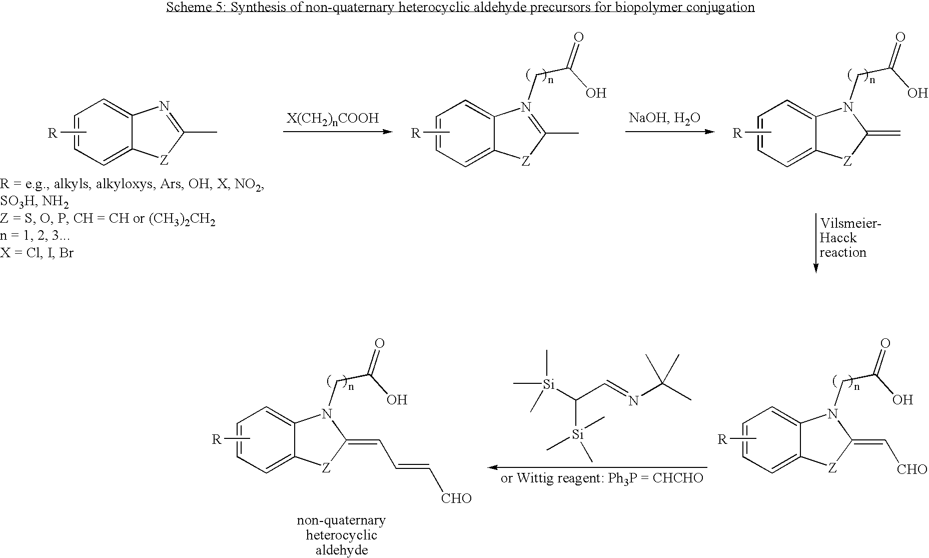

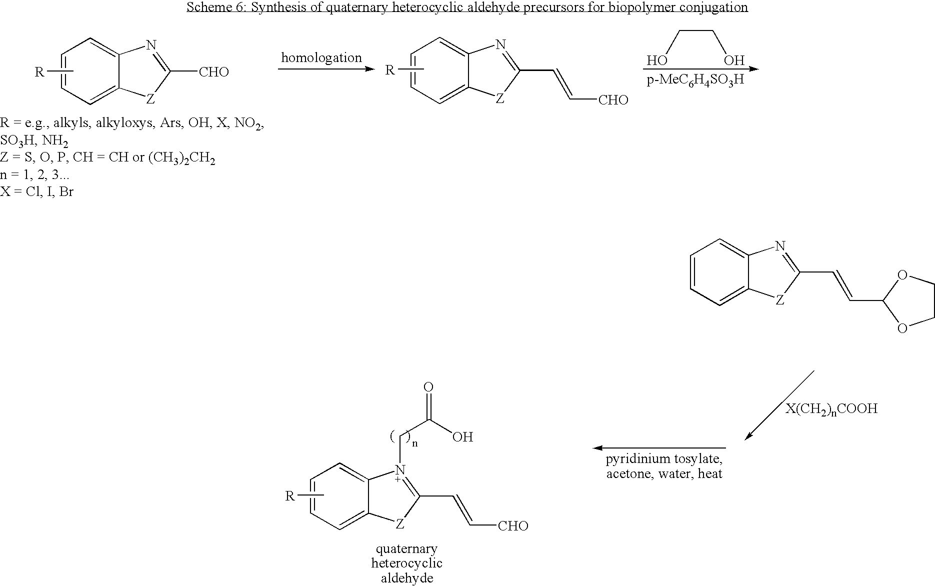

(iv) Synthesis of Precursors for Wittig or Horner Reaction

Heterocyclic triphenyl phosphine precursor can be conveniently linked to DNA through one of the phenyl groups. Scheme 7 provides a general method for synthesizing benzylic type phosphorane (Wittig reagent). The reactive halide is first synthesized from the corresponding benzylic alcohol and then reacts with 4-(diphenylphosphino)benzoic acid to form the phosphonium salt. For synthesizing some special amino substituted aromatic phosphonium salt, a convenient one-pot procedure without isolation of halide reagent was used (Scheme 8, Porrès, et al., Synthesis 2003, 10, 1541-1544). For synthesizing specifically Wittig reagents for cyanine, however, there are few challenges. First, it is difficult to obtain heterocyclic phosphonium salt precursor. Secondly, little is known about the reactivities of these reagents toward aldehyde.

Scheme 9 describes a general methodology for synthesis non-quaternary heterocyclic phosphorane. Alternative phosphonate reagent is also proposed here for Horner reaction (Scheme 10).

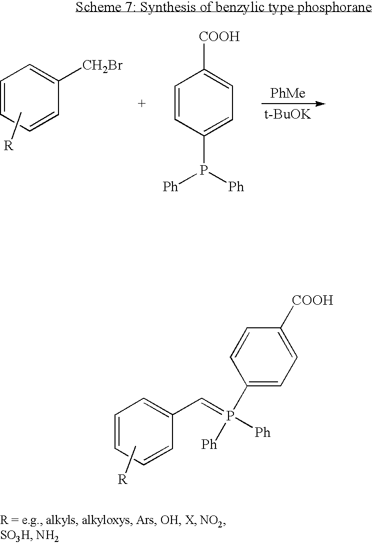

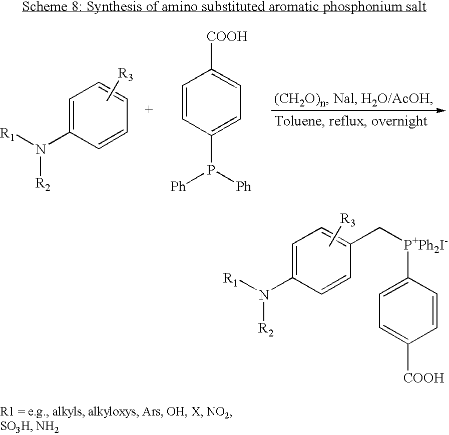

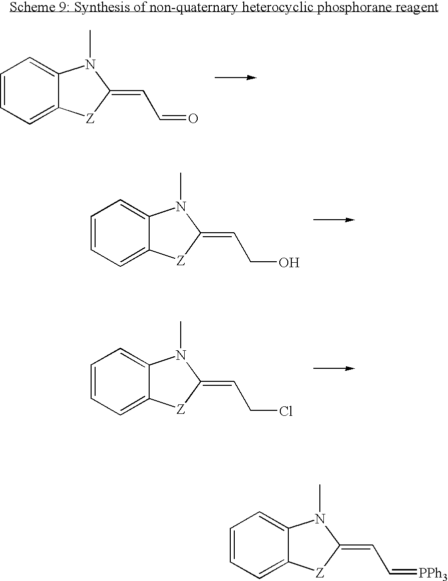

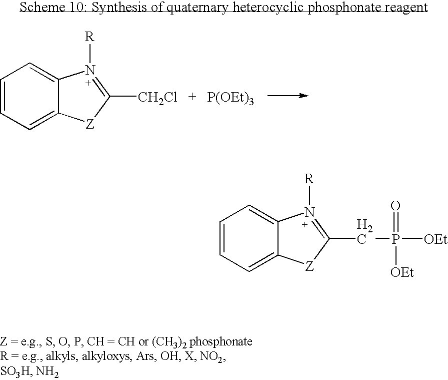

(v) Synthesis of Heterocyclic Precursors Bearing Active-Hydrogen for Aldol Condensation

Most of the heterocyclic precursors bearing active-hydrogen such as methyl group are commercially available. The acid functionality can be easily introduced to these compounds through N-quaternization (Scheme 11).

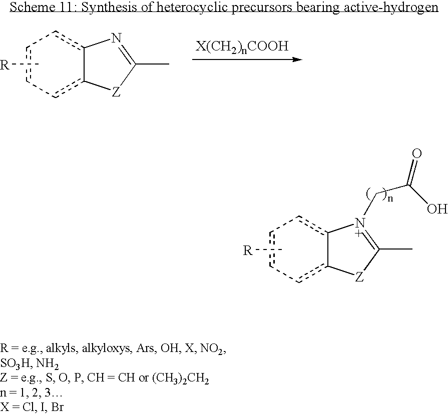

(vi) Polymethine Generation Through Nucleic Acid-Templated Wittig Reaction

Scheme 12 and Scheme 13 illustrate polymethine dye synthesis through nucleic acid-templated reactions including Wittig reaction and aldol condensation. For nucleic acid-templated Wittig reaction, a fluorescence polymethine dye conjugated single-strand DNA is generated with non-fluorescence phosphine oxide conjugated to other DNA strand. For aldol condensation, the polymethine dye is covalently linked to both DNA strands. They provide useful reporter chemistry and a method for the homogeneous fluorescence assay of biological system both in vitro and in vivo.

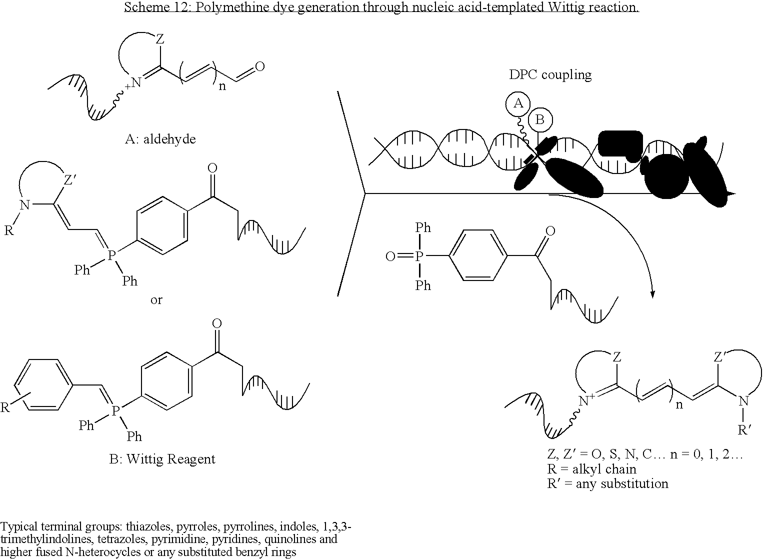

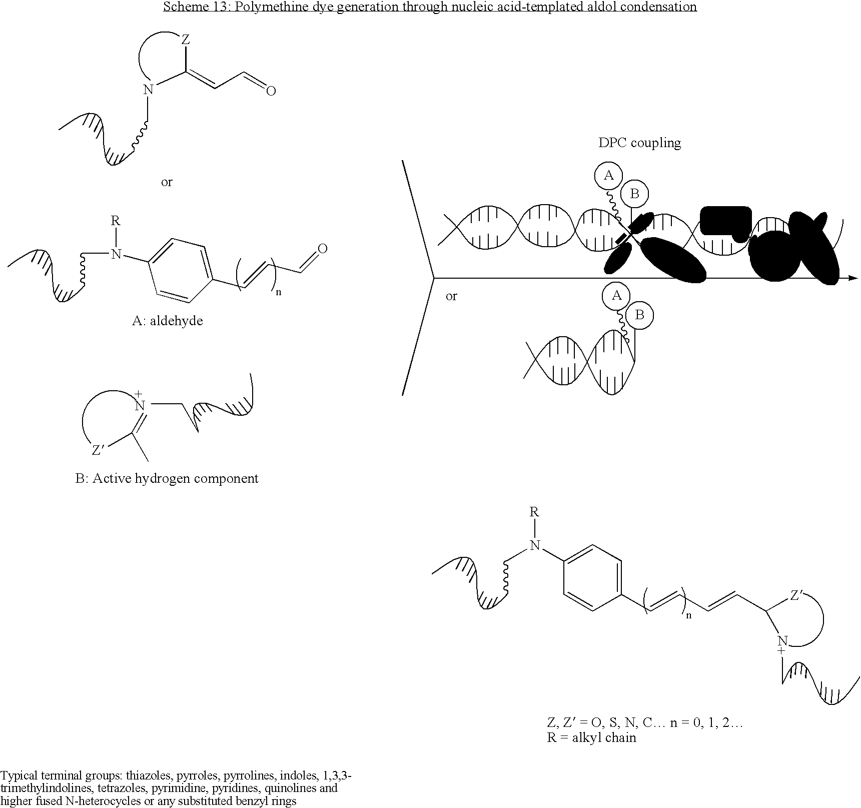

A variety of polymethine dyes may be generated (range from near UV to near IR) via nucleic acid-templated reactions. Since nucleic acid-templated chemistry is based on Watson-Crick base pairing, a multi-dye system can be established by using multi DNA probes attached with different polymethine dye precursors.

Chemical Reactions Useful in Biodetection Employing Nucleic Acid-Templated Chemistry

(i) Coupling Reactions

The reactive groups may be, for example, electrophiles (e.g., acetyl, amides, acid chlorides, esters, nitriles, imines), nucleophiles (e.g., amines, hydroxyl groups, thiols), catalysts (e.g., organometallic catalysts), or side chains.

(ii) Functional Group Transformations

Nucleic acid-templated chemistry can be used to effect functional group transformations that either (i) unmask or (ii) interconvert functionality used in coupling reactions, (iii) interconversions of functional groups present on a reactive group.

(iii) Reaction Conditions

Nucleic acid-templated reactions can occur in aqueous or non-aqueous (i.e., organic) solutions, or a mixture of one or more aqueous and non-aqueous solutions. Reaction conditions preferably are optimized to suit the nature of the reactive groups, oligonucleotides used, and the sample detection conditions.

(iv) Classes of Chemical Reactions

Known chemical reactions can be considered for use in nucleic acid-templated reactions, e.g., reactions such as those listed in March's Advanced Organic Chemistry, Organic Reactions, Organic Syntheses, organic text books, journals such as Journal of the American Chemical Society, Journal of Organic Chemistry, Tetrahedron, etc., and Carruther's Some Modern Methods of Organic Chemistry. The chosen reactions should be compatible with nucleic acids such as DNA or RNA or are compatible with the detection environment.

Reactions useful in nucleic-acid templated chemistry include, for example, substitution reactions, carbon-carbon bond forming reactions, elimination reactions, acylation reactions, and addition reactions. An illustrative but not exhaustive list of aliphatic nucleophilic substitution reactions useful in the present invention includes, for example, S_{N}2 reactions, S_{N}1 reactions, S_{N}i reactions, allylic rearrangements, nucleophilic substitution at an aliphatic trigonal carbon, and nucleophilic substation at a vinylic carbon.

Specific aliphatic nucleophilic substitution reactions with oxygen nucleophiles include, for example, hydrolysis of alkyl halides, hydrolysis of gen-dihalides, hydrolysis of 1,1,1-trihalides, hydrolysis of alkyl esters or inorganic acids, hydrolysis of diazo ketones, hydrolysis of acetal and enol ethers, hydrolysis of epoxides, hydrolysis of acyl halides, hydrolysis of anhydrides, hydrolysis of carboxylic esters, hydrolysis of amides, alkylation with alkyl halides (Williamson Reaction), epoxide formation, alkylation with inorganic esters, alkylation with diazo compounds, dehydration of alcohols, transetherification, alcoholysis of epoxides, alkylation with onium salts, hydroxylation of silanes, alcoholysis of acyl halides, alcoholysis of anhydrides, esterfication of carboxylic acids, alcoholysis of carboxylic esters (transesterfication), alcoholysis of amides, alkylation of carboxylic acid salts, cleavage of ether with acetic anhydride, alkylation of carboxylic acids with diazo compounds, acylation of caroxylic acids with acyl halides, acylation of carboxylic acids with carboxylic acids, formation of oxonium salts, preparation of peroxides and hydroperoxides, preparation of inorganic esters (e.g., nitrites, nitrates, sulfonates), preparation of alcohols from amines, and preparation of mixed organic-inorganic anhydrides.

Specific aliphatic nucleophilic substitution reactions with sulfur nucleophiles, which tend to be better nucleophiles than their oxygen analogs, include, for example, attack by SH at an alkyl carbon to form thiols, attack by S at an alkyl carbon to form thioethers, attack by SH or SR at an acyl carbon, formation of disulfides, formation of Bunte salts, alkylation of sulfinic acid salts, and formation of alkyl thiocyanates.

Aliphatic nucleophilic substitution reactions with nitrogen nucleophiles include, for example, alkylation of amines, N-arylation of amines, replacement of a hydroxy by an amino group, transamination, transamidation, alkylation of amines with diazo compounds, amination of epoxides, amination of oxetanes, amination of aziridines, amination of alkanes, formation of isocyanides, acylation of amines by acyl halides, acylation of amines by anhydrides, acylation of amines by carboxylic acids, acylation of amines by carboxylic esters, acylation of amines by amides, acylation of amines by other acid derivatives, N-alkylation or N-arylation of amides and imides, N-acylation of amides and imides, formation of aziridines from epoxides, formation of nitro compounds, formation of azides, formation of isocyanates and isothiocyanates, and formation of azoxy compounds.

Aliphatic nucleophilic substitution reactions with halogen nucleophiles include, for example, attack at an alkyl carbon, halide exchange, formation of alkyl halides from esters of sulfuric and sulfonic acids, formation of alkyl halides from alcohols, formation of alkyl halides from ethers, formation of halohydrins from epoxides, cleavage of carboxylic esters with lithium iodide, conversion of diazo ketones to α-halo ketones, conversion of amines to halides, conversion of tertiary amines to cyanamides (the von Braun reaction), formation of acyl halides from carboxylic acids, and formation of acyl halides from acid derivatives.

Aliphatic nucleophilic substitution reactions using hydrogen as a nucleophile include, for example, reduction of alkyl halides, reduction of tosylates, other sulfonates, and similar compounds, hydrogenolysis of alcohols, hydrogenolysis of esters (Barton-McCombie reaction), hydrogenolysis of nitriles, replacement of alkoxyl by hydrogen, reduction of epoxides, reductive cleavage of carboxylic esters, reduction of a C—N bond, desulfurization, reduction of acyl halides, reduction of carboxylic acids, esters, and anhydrides to aldehydes, and reduction of amides to aldehydes.

Although certain carbon nucleophiles may be too nucleophilic and/or basic to be used in certain embodiments of the invention, aliphatic nucleophilic substitution reactions using carbon nucleophiles include, for example, coupling with silanes, coupling of alkyl halides (the Wurtz reaction), the reaction of alkyl halides and sulfonate esters with Group I (I A) and II (II A) organometallic reagents, reaction of alkyl halides and sulfonate esters with organocuprates, reaction of alkyl halides and sulfonate esters with other organometallic reagents, allylic and propargylic coupling with a halide substrate, coupling of organometallic reagents with esters of sulfuric and sulfonic acids, sulfoxides, and sulfones, coupling involving alcohols, coupling of organometallic reagents with carboxylic esters, coupling of organometallic reagents with compounds containing an esther linkage, reaction of organometallic reagents with epoxides, reaction of organometallics with aziridine, alkylation at a carbon bearing an active hydrogen, alkylation of ketones, nitriles, and carboxylic esters, alkylation of carboxylic acid salts, alkylation at a position a to a heteroatom (alkylation of 1,3-dithianes), alkylation of dihydro-1,3-oxazine (the Meyers synthesis of aldehydes, ketones, and carboxylic acids), alkylation with trialkylboranes, alkylation at an alkynyl carbon, preparation of nitriles, direct conversion of alkyl halides to aldehydes and ketones, conversion of alkyl halides, alcohols, or alkanes to carboxylic acids and their derivatives, the conversion of acyl halides to ketones with organometallic compounds, the conversion of anhydrides, carboxylic esters, or amides to ketones with organometallic compounds, the coupling of acyl halides, acylation at a carbon bearing an active hydrogen, acylation of carboxylic esters by carboxylic esters (the Claisen and Dieckmann condensation), acylation of ketones and nitriles with carboxylic esters, acylation of carboxylic acid salts, preparation of acyl cyanides, and preparation of diazo ketones, ketonic decarboxylation.

Reactions which involve nucleophilic attack at a sulfonyl sulfur atom may also be used in the present invention and include, for example, hydrolysis of sulfonic acid derivatives (attack by OH), formation of sulfonic esters (attack by OR), formation of sulfonamides (attack by nitrogen), formation of sulfonyl halides (attack by halides), reduction of sulfonyl chlorides (attack by hydrogen), and preparation of sulfones (attack by carbon).

Aromatic electrophilic substitution reactions may also be used in nucleotide-templated chemistry. Hydrogen exchange reactions are examples of aromatic electrophilic substitution reactions that use hydrogen as the electrophile. Aromatic electrophilic substitution reactions which use nitrogen electrophiles include, for example, nitration and nitro-de-hydrogenation, nitrosation of nitroso-de-hydrogenation, diazonium coupling, direct introduction of the diazonium group, and amination or amino-de-hydrogenation. Reactions of this type with sulfur electropliles include, for example, sulfonation, sulfo-de-hydrogenation, halosulfonation, halosulfo-de-hydrogenation, sulfurization, and sulfonylation. Reactions using halogen electrophiles include, for example, halogenation, and halo-de-hydrogenation. Aromatic electrophilic substitution reactions with carbon electrophiles include, for example, Friedel-Crafts alkylation, alkylation, alkyl-de-hydrogenation, Friedel-Crafts arylation (the Scholl reaction), Friedel-Crafts acylation, formylation with disubstituted formamides, formylation with zinc cyanide and HCl (the Gatterman reaction), formylation with chloroform (the Reimer-Tiemann reaction), other formylations, formyl-de-hydrogenation, carboxylation with carbonyl halides, carboxylation with carbon dioxide (the Kolbe-Schmitt reaction), amidation with isocyanates, N-alkylcarbamoyl-de-hydrogenation, hydroxyalkylation, hydroxyalkyl-de-hydrogenation, cyclodehydration of aldehydes and ketones, haloalkylation, halo-de-hydrogenation, aminoalkylation, amidoalkylation, dialkylaminoalkylation, dialkylamino-de-hydrogenation, thioalkylation, acylation with nitriles (the Hoesch reaction), cyanation, and cyano-de-hydrogenation. Reactions using oxygen electrophiles include, for example, hydroxylation and hydroxy-de-hydrogenation.

Rearrangement reactions include, for example, the Fries rearrangement, migration of a nitro group, migration of a nitroso group (the Fischer-Hepp Rearrangement), migration of an arylazo group, migration of a halogen (the Orton rearrangement), migration of an alkyl group, etc. Other reaction on an aromatic ring include the reversal of a Friedel-Crafts alkylation, decarboxylation of aromatic aldehydes, decarboxylation of aromatic acids, the Jacobsen reaction, deoxygenation, desulfonation, hydro-de-sulfonation, dehalogenation, hydro-de-halogenation, and hydrolysis of organometallic compounds.

Aliphatic electrophilic substitution reactions are also useful. Reactions using the S_{E}1, S_{E}2 (front), S_{E}2 (back), S_{E}i, addition-elimination, and cyclic mechanisms can be used in the present invention. Reactions of this type with hydrogen as the leaving group include, for example, hydrogen exchange (deuterio-de-hydrogenation, deuteriation), migration of a double bond, and keto-enol tautomerization. Reactions with halogen electrophiles include, for example, halogenation of aldehydes and ketones, halogenation of carboxylic acids and acyl halides, and halogenation of sulfoxides and sulfones. Reactions with nitrogen electrophiles include, for example, aliphatic diazonium coupling, nitrosation at a carbon bearing an active hydrogen, direct formation of diazo compounds, conversion of amides to α-azido amides, direct amination at an activated position, and insertion by nitrenes. Reactions with sulfur or selenium electrophiles include, for example, sulfenylation, sulfonation, and selenylation of ketones and carboxylic esters. Reactions with carbon electrophiles include, for example, acylation at an aliphatic carbon, conversion of aldehydes to β-keto esters or ketones, cyanation, cyano-de-hydrogenation, alkylation of alkanes, the Stork enamine reaction, and insertion by carbenes. Reactions with metal electrophiles include, for example, metalation with organometallic compounds, metalation with metals and strong bases, and conversion of enolates to silyl enol ethers. Aliphatic electrophilic substitution reactions with metals as leaving groups include, for example, replacement of metals by hydrogen, reactions between organometallic reagents and oxygen, reactions between organometallic reagents and peroxides, oxidation of trialkylboranes to borates, conversion of Grignard reagents to sulfur compounds, halo-de-metalation, the conversion of organometallic compounds to amines, the conversion of organometallic compounds to ketones, aldehydes, carboxylic esters and amides, cyano-de-metalation, transmetalation with a metal, transmetalation with a metal halide, transmetalation with an organometallic compound, reduction of alkyl halides, metallo-de-halogenation, replacement of a halogen by a metal from an organometallic compound, decarboxylation of aliphatic acids, cleavage of alkoxides, replacement of a carboxyl group by an acyl group, basic cleavage of β-keto esters and β-diketones, haloform reaction, cleavage of non-enolizable ketones, the Haller-Bauer reaction, cleavage of alkanes, decyanation, and hydro-de-cyanation. Electrophlic substitution reactions at nitrogen include, for example, diazotization, conversion of hydrazines to azides, N-nitrosation, N-nitroso-de-hydrogenation, conversion of amines to azo compounds, N-halogenation, N-halo-de-hydrogenation, reactions of amines with carbon monoxide, and reactions of amines with carbon dioxide.

Aromatic nucleophilic substitution reactions may also be used in the present invention. Reactions proceeding via the S_{N}Ar mechanism, the S_{N}1 mechanism, the benzyne mechanism, the S_{RN}1 mechanism, or other mechanism, for example, can be used. Aromatic nucleophilic substitution reactions with oxygen nucleophiles include, for example, hydroxy-de-halogenation, alkali fusion of sulfonate salts, and replacement of OR or OAr. Reactions with sulfur nucleophiles include, for example, replacement by SH or SR. Reactions using nitrogen nucleophiles include, for example, replacement by NH₂, NHR, or NR₂, and replacement of a hydroxy group by an amino group. Reactions with halogen nucleophiles include, for example, the introduction halogens. Aromatic nucleophilic substitution reactions with hydrogen as the nucleophile include, for example, reduction of phenols and phenolic esters and ethers, and reduction of halides and nitro compounds. Reactions with carbon nucleophiles include, for example, the Rosenmund-von Braun reaction, coupling of organometallic compounds with aryl halides, ethers, and carboxylic esters, arylation at a carbon containing an active hydrogen, conversions of aryl substrates to carboxylic acids, their derivatives, aldehydes, and ketones, and the Ullmann reaction. Reactions with hydrogen as the leaving group include, for example, alkylation, arylation, and amination of nitrogen heterocycles. Reactions with N₂⁺ as the leaving group include, for example, hydroxy-de-diazoniation, replacement by sulfur-containing groups, iodo-de-diazoniation, and the Schiemann reaction. Rearrangement reactions include, for example, the von Richter rearrangement, the Sommelet-Hauser rearrangement, rearrangement of aryl hydroxylamines, and the Smiles rearrangement.

Reactions involving free radicals can also be used, although the free radical reactions used in nucleotide-templated chemistry should be carefully chosen to avoid modification or cleavage of the nucleotide template. With that limitation, free radical substitution reactions can be used in the present invention. Particular free radical substitution reactions include, for example, substitution by halogen, halogenation at an alkyl carbon, allylic halogenation, benzylic halogenation, halogenation of aldehydes, hydroxylation at an aliphatic carbon, hydroxylation at an aromatic carbon, oxidation of aldehydes to carboxylic acids, formation of cyclic ethers, formation of hydroperoxides, formation of peroxides, acyloxylation, acyloxy-de-hydrogenation, chlorosulfonation, nitration of alkanes, direct conversion of aldehydes to amides, amidation and amination at an alkyl carbon, simple coupling at a susceptible position, coupling of alkynes, arylation of aromatic compounds by diazonium salts, arylation of activated alkenes by diazonium salts (the Meerwein arylation), arylation and alkylation of alkenes by organopalladium compounds (the Heck reaction), arylation and alkylation of alkenes by vinyltin compounds (the Stille reaction), alkylation and arylation of aromatic compounds by peroxides, photochemical arylation of aromatic compounds, alkylation, acylation, and carbalkoxylation of nitrogen heterocycles Particular reactions in which N₂⁺ is the leaving group include, for example, replacement of the diazonium group by hydrogen, replacement of the diazonium group by chlorine or bromine, nitro-de-diazoniation, replacement of the diazonium group by sulfur-containing groups, aryl dimerization with diazonium salts, methylation of diazonium salts, vinylation of diazonium salts, arylation of diazonium salts, and conversion of diazonium salts to aldehydes, ketones, or carboxylic acids. Free radical substitution reactions with metals as leaving groups include, for example, coupling of Grignard reagents, coupling of boranes, and coupling of other organometallic reagents. Reaction with halogen as the leaving group are included. Other free radical substitution reactions with various leaving groups include, for example, desulfurization with Raney Nickel, conversion of sulfides to organolithium compounds, decarboxylative dimerization (the Kolbe reaction), the Hunsdiecker reaction, decarboxylative allylation, and decarbonylation of aldehydes and acyl halides.

Reactions involving additions to carbon-carbon multiple bonds are also used in nucleotide-templated chemistry. Any mechanism may be used in the addition reaction including, for example, electrophilic addition, nucleophilic addition, free radical addition, and cyclic mechanisms. Reactions involving additions to conjugated systems can also be used. Addition to cyclopropane rings can also be utilized. Particular reactions include, for example, isomerization, addition of hydrogen halides, hydration of double bonds, hydration of triple bonds, addition of alcohols, addition of carboxylic acids, addition of H₂S and thiols, addition of ammonia and amines, addition of amides, addition of hydrazoic acid, hydrogenation of double and triple bonds, other reduction of double and triple bonds, reduction of the double and triple bonds of conjugated systems, hydrogenation of aromatic rings, reductive cleavage of cyclopropanes, hydroboration, other hydrometalations, addition of alkanes, addition of alkenes and/or alkynes to alkenes and/or alkynes (e.g., pi-cation cyclization reactions, hydro-alkenyl-addition), ene reactions, the Michael reaction, addition of organometallics to double and triple bonds not conjugated to carbonyls, the addition of two alkyl groups to an alkyne, 1,4-addition of organometallic compounds to activated double bonds, addition of boranes to activated double bonds, addition of tin and mercury hydrides to activated double bonds, acylation of activated double bonds and of triple bonds, addition of alcohols, amines, carboxylic esters, aldehydes, etc., carbonylation of double and triple bonds, hydrocarboxylation, hydroformylation, addition of aldehydes, addition of HCN, addition of silanes, radical addition, radical cyclization, halogenation of double and triple bonds (addition of halogen, halogen), halolactonization, halolactamization, addition of hypohalous acids and hypohalites (addition of halogen, oxygen), addition of sulfur compounds (addition of halogen, sulfur), addition of halogen and an amino group (addition of halogen, nitrogen), addition of NOX and NO₂X (addition of halogen, nitrogen), addition of XN₃ (addition of halogen, nitrogen), addition of alkyl halides (addition of halogen, carbon), addition of acyl halides (addition of halogen, carbon), hydroxylation (addition of oxygen, oxygen) (e.g., asymmetric dihydroxylation reaction with OSO₄), dihydroxylation of aromatic rings, epoxidation (addition of oxygen, oxygen) (e.g., Sharpless asymmetric epoxidation), photooxidation of dienes (addition of oxygen, oxygen), hydroxysulfenylation (addition of oxygen, sulfur), oxyamination (addition of oxygen, nitrogen), diamination (addition of nitrogen, nitrogen), formation of aziridines (addition of nitrogen), aminosulfenylation (addition of nitrogen, sulfur), acylacyloxylation and acylamidation (addition of oxygen, carbon or nitrogen, carbon), 1,3-dipolar addition (addition of oxygen, nitrogen, carbon), Diels-Alder reaction, heteroatom Diels-Alder reaction, all carbon 3+2 cycloadditions, dimerization of alkenes, the addition of carbenes and carbenoids to double and triple bonds, trimerization and tetramerization of alkynes, and other cycloaddition reactions.

In addition to reactions involving additions to carbon-carbon multiple bonds, addition reactions to carbon-hetero multiple bonds can be used in nucleotide-templated chemistry. Exemplary reactions include, for example, the addition of water to aldehydes and ketones (formation of hydrates), hydrolysis of carbon-nitrogen double bond, hydrolysis of aliphatic nitro compounds, hydrolysis of nitriles, addition of alcohols and thiols to aldehydes and ketones, reductive alkylation of alcohols, addition of alcohols to isocyanates, alcoholysis of nitriles, formation of xanthates, addition of H₂S and thiols to carbonyl compounds, formation of bisulfite addition products, addition of amines to aldehydes and ketones, addition of amides to aldehydes, reductive alkylation of ammonia or amines, the Mannich reaction, the addition of amines to isocyanates, addition of ammonia or amines to nitriles, addition of amines to carbon disulfide and carbon dioxide, addition of hydrazine derivative to carbonyl compounds, formation of oximes, conversion of aldehydes to nitriles, formation of gem-dihalides from aldehydes and ketones, reduction of aldehydes and ketones to alcohols, reduction of the carbon-nitrogen double bond, reduction of nitriles to anines, reduction of nitriles to aldehydes, addition of Grignard reagents and organolithium reagents to aldehydes and ketones, addition of other organometallics to aldehydes and ketones, addition of trialkylallylsilanes to aldehydes and ketones, addition of conjugated alkenes to aldehydes (the Baylis-Hillman reaction), the Reformatsky reaction, the conversion of carboxylic acid salts to ketones with organometallic compounds, the addition of Grignard reagents to acid derivatives, the addition of organometallic compounds to CO₂ and CS₂, addition of organometallic compounds to C═N compounds, addition of carbenes and diazoalkanes to C═N compounds, addition of Grignard reagents to nitriles and isocyanates, the Aldol reaction, Mukaiyama Aldol and related reactions, Aldol-type reactions between carboxylic esters or amides and aldehydes or ketones, the Knoevenagel reaction (e.g., the Nef reaction, the Favorskii reaction), the Peterson alkenylation reaction, the addition of active hydrogen compounds to CO₂ and CS₂, the Perkin reaction, Darzens glycidic ester condensation, the Tollens' reaction, the Wittig reaction, the Tebbe alkenylation, the Petasis alkenylation, alternative alkenylations, the Thorpe reaction, the Thorpe-Ziegler reaction, addition of silanes, formation of cyanohydrins, addition of HCN to C═N and C═N bonds, the Prins reaction, the benzoin condensation, addition of radicals to C═O, C═S, C═N compounds, the Ritter reaction, acylation of aldehydes and ketones, addition of aldehydes to aldehydes, the addition of isocyanates to isocyanates (formation of carbodiimides), the conversion of carboxylic acid salts to nitriles, the formation of epoxides from aldehydes and ketones, the formation of episulfides and episulfones, the formation of β-lactones and oxetanes (e.g., the Patemo-Büchi reaction), the formation of β-lactams, etc. Reactions involving addition to isocyanides include the addition of water to isocyanides, the Passerini reaction, the Ug reaction, and the formation of metalated aldimines.

Elimination reactions, including α, β, and γ eliminations, as well as extrusion reactions, can be performed using nucleotide-templated chemistry, although the strength of the reagents and conditions employed should be considered. Preferred elimination reactions include reactions that go by E1, E2, E1cB, or E2C mechanisms. Exemplary reactions include, for example, reactions in which hydrogen is removed from one side (e.g., dehydration of alcohols, cleavage of ethers to alkenes, the Chugaev reaction, ester decomposition, cleavage of quarternary ammonium hydroxides, cleavage of quaternary ammonium salts with strong bases, cleavage of amine oxides, pyrolysis of keto-ylids, decomposition of toluene-p-solfonylhydrazones, cleavage of sulfoxides, cleavage of selenoxides, cleavage of sulfornes, dehydrogalogenation of alkyl halides, dehydrohalogenation of acyl halides, dehydrohalogenation of sulfonyl halides, elimination of boranes, conversion of alkenes to alkynes, decarbonylation of acyl halides), reactions in which neither leaving atom is hydrogen (e.g., deoxygenation of vicinal diols, cleavage of cyclic thionocarbonates, conversion of epoxides to episulfides and alkenes, the Ramberg-Bäcklund reaction, conversion of aziridines to alkenes, dehalogenation of vicinal dihalides, dehalogenation of α-halo acyl halides, and elimination of a halogen and a hetero group), fragmentation reactions (i.e., reactions in which carbon is the positive leaving group or the electrofuge, such as, for example, fragmentation of γ-amino and γ-hydroxy halides, fragmentation of 1,3-diols, decarboxylation of β-hydroxy carboxylic acids, decarboxylation of β-lactones, fragmentation of α,β-epoxy hydrazones, elimination of CO from bridged bicyclic compounds, and elimination of CO₂ from bridged bicyclic compounds), reactions in which C≡N or C═N bonds are formed (e.g., dehydration of aldoximes or similar compounds, conversion of ketoximes to nitriles, dehydration of unsubstituted amides, and conversion of N-alkylformamides to isocyanides), reactions in which C═O bonds are formed (e.g., pyrolysis of β-hydroxy alkenes), and reactions in which N═N bonds are formed (e.g., eliminations to give diazoalkenes). Extrusion reactions include, for example, extrusion of N₂ from pyrazolines, extrusion of N₂ from pyrazoles, extrusion of N₂ from triazolines, extrusion of CO, extrusion of CO₂, extrusion of SO₂, the Story synthesis, and alkene synthesis by twofold extrusion.

Rearrangements, including, for example, nucleophilic rearrangements, electrophilic rearrangements, prototropic rearrangements, and free-radical rearrangements, can also be performed using nucleotide-templated chemistry. Both 1,2 rearrangements and non-1,2 rearrangements can be performed. Exemplary reactions include, for example, carbon-to-carbon migrations of R, H, and Ar (e.g., Wagner-Meerwein and related reactions, the Pinacol rearrangement, ring expansion reactions, ring contraction reactions, acid-catalyzed rearrangements of aldehydes and ketones, the dienone-phenol rearrangement, the Favorskii rearrangement, the Arndt-Eistert synthesis, homologation of aldehydes, and homologation of ketones), carbon-to-carbon migrations of other groups (e.g., migrations of halogen, hydroxyl, amino, etc.; migration of boron; and the Neber rearrangement), carbon-to-nitrogen migrations of R and Ar (e.g., the Hofmann rearrangement, the Curtius rearrangement, the Lossen rearrangement, the Schmidt reaction, the Beckman rearrangement, the Stieglits rearrangement, and related rearrangements), carbon-to-oxygen migrations of R and Ar (e.g., the Baeyer-Villiger rearrangement and rearrangment of hydroperoxides), nitrogen-to-carbon, oxygen-to-carbon, and sulfur-to-carbon migration (e.g., the Stevens rearrangement, and the Wittig rearrangement), boron-to-carbon migrations (e.g., conversion of boranes to alcohols (primary or otherwise), conversion of boranes to aldehydes, conversion of boranes to carboxylic acids, conversion of vinylic boranes to alkenes, formation of alkynes from boranes and acetylides, formation of alkenes from boranes and acetylides, and formation of ketones from boranes and acetylides), electrocyclic rearrangements (e.g., of cyclobutenes and 1,3-cyclohexadienes, or conversion of stilbenes to phenanthrenes), sigmatropic rearrangements (e.g., (1,j) sigmatropic migrations of hydrogen, (1,j) sigmatropic migrations of carbon, conversion of vinylcyclopropanes to cyclopentenes, the Cope rearrangement, the Claisen rearrangement, the Fischer indole synthesis, (2,3) sigmatropic rearrangements, and the benzidine rearrangement), other cyclic rearrangements (e.g., metathesis of alkenes, the di-π-methane and related rearrangements, and the Hofmann-Löffler and related reactions), and non-cyclic rearrangements (e.g., hydride shifts, the Chapman rearrangement, the Wallach rearrangement, and dyotropic rearrangements).

Oxidative and reductive reactions may also be performed using nucleotide-templated chemistry. Exemplary reactions may involve, for example, direct electron transfer, hydride transfer, hydrogen-atom transfer, formation of ester intermediates, displacement mechanisms, or addition-elimination mechanisms. Exemplary oxidations include, for example, eliminations of hydrogen (e.g., aromatization of six-membered rings, dehydrogenations yielding carbon-carbon double bonds, oxidation or dehydrogenation of alcohols to aldehydes and ketones, oxidation of phenols and aromatic amines to quinones, oxidative cleavage of ketones, oxidative cleavage of aldehydes, oxidative cleavage of alcohols, ozonolysis, oxidative cleavage of double bonds and aromatic rings, oxidation of aromatic side chains, oxidative decarboxylation, and bisdecarboxylation), reactions involving replacement of hydrogen by oxygen (e.g., oxidation of methylene to carbonyl, oxidation of methylene to OH, CO₂R, or OR, oxidation of arylmethanes, oxidation of ethers to carboxylic esters and related reactions, oxidation of aromatic hydrocarbons to quinones, oxidation of amines or nitro compounds to aldehydes, ketones, or dihalides, oxidation of primary alcohols to carboxylic acids or carboxylic esters, oxidation of alkenes to aldehydes or ketones, oxidation of amines to nitroso compounds and hydroxylamines, oxidation of primary amines, oximes, azides, isocyanates, or notroso compounds, to nitro compounds, oxidation of thiols and other sulfur compounds to sulfonic acids), reactions in which oxygen is added to the subtrate (e.g., oxidation of alkynes to α-diketones, oxidation of tertiary amines to amine oxides, oxidation of thioesters to sulfoxides and sulfones, and oxidation of carboxylic acids to peroxy acids), and oxidative coupling reactions (e.g., coupling involving carbanoins, dimerization of silyl enol ethers or of lithium enolates, and oxidation of thiols to disulfides).

Exemplary reductive reactions include, for example, reactions involving replacement of oxygen by hydrogen (e.g., reduction of carbonyl to methylene in aldehydes and ketones, reduction of carboxylic acids to alcohols, reduction of amides to amines, reduction of carboxylic esters to ethers, reduction of cyclic anhydrides to lactones and acid derivatives to alcohols, reduction of carboxylic esters to alcohols, reduction of carboxylic acids and esters to alkanes, complete reduction of epoxides, reduction of nitro compounds to amines, reduction of nitro compounds to hydroxylamines, reduction of nitroso compounds and hydroxylamines to amines, reduction of oximes to primary amines or aziridines, reduction of azides to primary amines, reduction of nitrogen compounds, and reduction of sulfonyl halides and sulfonic acids to thiols), removal of oxygen from the substrate (e.g., reduction of amine oxides and azoxy compounds, reduction of sulfoxides and sulfones, reduction of hydroperoxides and peroxides, and reduction of aliphatic nitro compounds to oximes or nitriles), reductions that include cleavage (e.g., de-alkylation of amines and amides, reduction of azo, azoxy, and hydrazo compounds to amines, and reduction of disulfides to thiols), reductive couplic reactions (e.g., bimolecular reduction of aldehydes and ketones to 1,2-diols, bimolecular reduction of aldehydes or ketones to alkenes, acyloin ester condensation, reduction of nitro to azoxy compounds, and reduction of nitro to azo compounds), and reductions in which an organic substrate is both oxidized and reduced (e.g., the Cannizzaro reaction, the Tishchenko reaction, the Pummerer rearrangement, and the Willgerodt reaction).

Various and general aspects of nucleic acid-templated chemistry are discussed in detail below. Additional information may be found in U.S. Patent Application Publication Nos. 2004/0180412 A1 (U.S. Ser. No. 10/643,752) by Liu et al. and 2003/0113738 A1 (U.S. Ser. No. 10/101,030) by Liu et al.

There are a number of advantages to the methods of signal creation encompassed by the invention disclosed here. For example, because the reactive moieties appended to the probes initially do not have detectable properties until a hybridization event (or in the case of non-nucleic acid targets, a hybridization event following a binding event) and subsequent reaction take place, assays employing probes and chemistries according to the invention have low to no background and therefore high signal-to-noise ratio. This in turn provides practical advantages of assays possessing high sensitivity and a wide dynamic range. Thus, smaller amounts of analyte may be detected with the potential to do so using detection instrumentation that is simpler and of lower cost. Many different types of signal generation (fluorescence generation, release of fluorescence, cofactor release etc.) can be supported through this mechanism.

An additional important practical advantage is that assays may be constructed so as to be homogeneous. Homogeneous assays require no or little sample preparation, nor do they typically require that analytes be immobilized on a solid-support for the purpose of reagent removal, background reduction, solvent or buffer exchange, and/or detection as is typically needed for heterogeneous assays. Because the formation of a double stranded DNA of high Tₘ is a homogeneous reaction, placing fluorophore precursors on the oligonucleotides supports an entirely homogenous phase assay for binding to the target. Formation of the double stranded structure itself is nearly instantaneous.

Another practical benefit of the invention is that probes and reagents can be added directly to the sample, and the resulting solution can be monitored for signal generation without any further manipulation such as attachment to solid-support, washing, etc. As a result this invention provides for very simple assays that can be performed in non-laboratory settings without the need for expensive or cumbersome equipment.

Because obtaining a double stranded DNA of high Tₘ normally requires the use of two separate binders to sites located as distances compatible with the spacer arms on the oligonucleotides, very high specificity of binding can be obtained.

Furthermore, the use of two binders which themselves become associated through the annealed DNAs should result in an enhanced affinity (avidity) effect. Therefore, two weak binders should exhibit an enhanced avidity of binding. Two binders, both of which may be weak but which have different specificity (binding to different sites) should exhibit enhanced avidity and specificity. This is highly advantages for low level detection when only weak binders are available.

The following examples contain important additional information, exemplification and guidance that can be adapted to the practice of this invention in its various embodiments and equivalents thereof. Practice of the invention will be more fully understood from these following examples, which are presented herein for illustrative purpose only, and should not be construed as limiting in anyway.

### EXAMPLES

### Example 1

### Creation of Fluorescence by Hybridization Induced Azidocoumarin Reduction

Five oligonucleotides were prepared using standard phosphoramidite chemistry (Glen Research, Sterling Va., USA). Oligonucleotides bearing 5′-amino groups (Oligo2 and Oligo6) were prepared using 5′-Amino-Modifier 5 and Oligonucleotides bearing 3′-amino groups (Oligo4 and Oligo5) were prepared using 3′-Amino-Modifier C7 CPG (Glen Research, Sterling Va., USA)

| | |
|---|---|
| Oligo1 | |
| 5′-GTGGTAGTTGGAGCTGGTGGCGTAGGCAA | (SEQ. ID. NO. 19) |
| GA-3′ | |
| Oligo2 | |
| 5′-H2N-AGCTCCAACTACCAC-3′ | (SEQ. ID. NO. 20) |
| Oligo4 | |
| 5′-GTGGTAGTTGGAGCT-NH2-3′ | (SEQ. ID. NO. 21) |
| Oligo5 | |
| 5′-TCTTGCCTACGCCAC-NH2-3′ | (SEQ. ID. NO. 22) |
| Oligo6 | |
| 5′-H2N-AGATCCCACTAGCAC-3′ | (SEQ. ID. NO. 23) |

Oligo1, Oligo4 and Oligo5 were removed from the synthesis support and purified by reversed-phase HPLC. The amino groups of Oligo2 and Oligo6 were converted while resin-bound to their triphenyl phosphine derivatives and these were purified and isolated (Sakurai et al., J. Amer. Chem. Soc. (2005) Vol. 127, pp 1660-1667) to give Oligo2-TPP and Oligo-6TPP, respectively.

Amino group bearing Oligo4 and Oligo5 were converted to their azidocoumarin derivatives (Oligo4-AzC and Oligo5-AzC, respectively) by reaction of each oligo with the N-hydroxysuccinimide ester of 7-azido-4-methylcoumarin-3-acetic acid (Thevenin et al., Eur. J. Biochem (1992) Vol. 206, pp-471-477). The reaction was performed by adding 1 uL of triflouroacetic acid to 5 uL of N-methylmorpholine to prepare a buffer to which was added 10 uL of water containing 6.6 nmol of Oligo 4 or Oligo 5, followed by addition of 30 uL of a 0.16 M solution of the coumarin NHS-ester in dimethylformamide. Each reaction was allowed to proceed for 2 hours at room temperature, whereupon 50 uL of 0.1 M aqueous triethylammonium acetate was added. The mixtures were applied to a NAP-5 desalting columns (Amersham Biosciences, Piscataway N.J. USA) and eluted according to the manufacturers instructions the eluate was purified by RP-HPLC to provide Oligo4-AzC and Oligo5-AzC, in yields of 77% and 70%, respectively. Product identity was confirmed by Maldi-ToF mass spectrometry.

To demonstrate the hybridization-specific creation of fluorescence, various combinations of complementary and non-complementary oligonucleotides bearing azido-coumarin and triphenyl phosphine moieties were allowed to react at room temperature in a buffer comprised of 30% aqueous formamide, 50 mM NaCl, and 10 mM sodium phosphate, pH 7.2. The reaction progress was monitored over time using a Victor Multilabel fluorimeter (EG&G Wallach, Turku Finlnad) set to excite the sample at 360 nm and monitor light emission at 455 nm

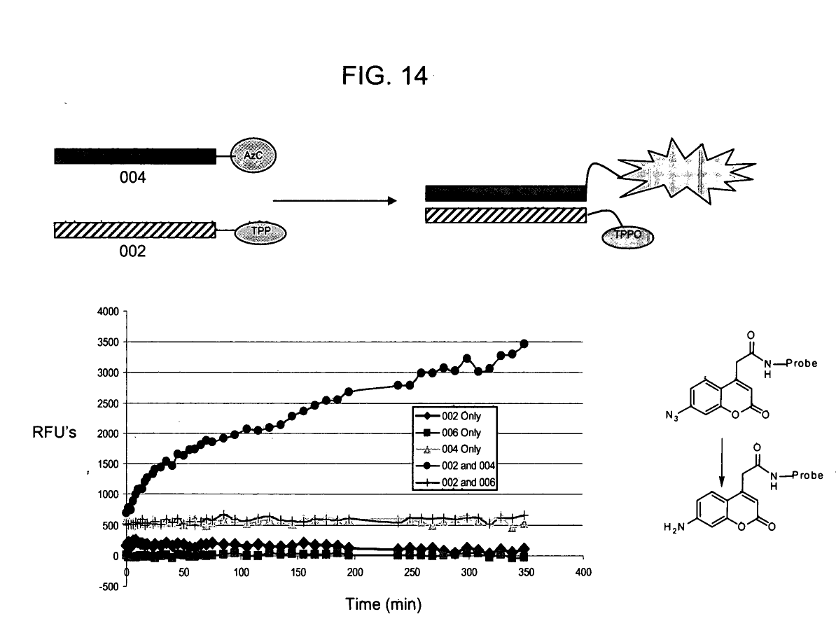

FIG. 14 shows that when Oligo4-AzC and Oligo2-TPP are combined to final concentrations of 200 nM and 400 nM respectively, a rapid increase in fluorescence is observed. In this figure 004 denotes Oligo4-AzC, 002 denote Oligo2-TPP, and 006 denotes Oligo6-TPP. The fluorescence does not occur when Oligo6-TPP is substituted for Oligo2-TPP. Whereas Oligo2-TPP is perfectly complementary in its base-pairing ability to Oligo4-AzC, Oligo6-TPP is not, as it contains three mismatched nucleotides. The results support the conclusion that the creation of fluorescence is due to the ability of Oligo2-TPP to hybridize to Oligo4-AzC thus facilitating a reaction between the TPP and azidocoumarin moieties in the resulting hybrid. The lack of signal in the case of reaction of Oligo6-TPP with Oligo4-AzC is consistent with inability of these two oligonucleotides to form a duplex, therefore the reaction is not facilitated. Control reactions containing each single oligonucleotide were performed to rule out any non-specific effects.

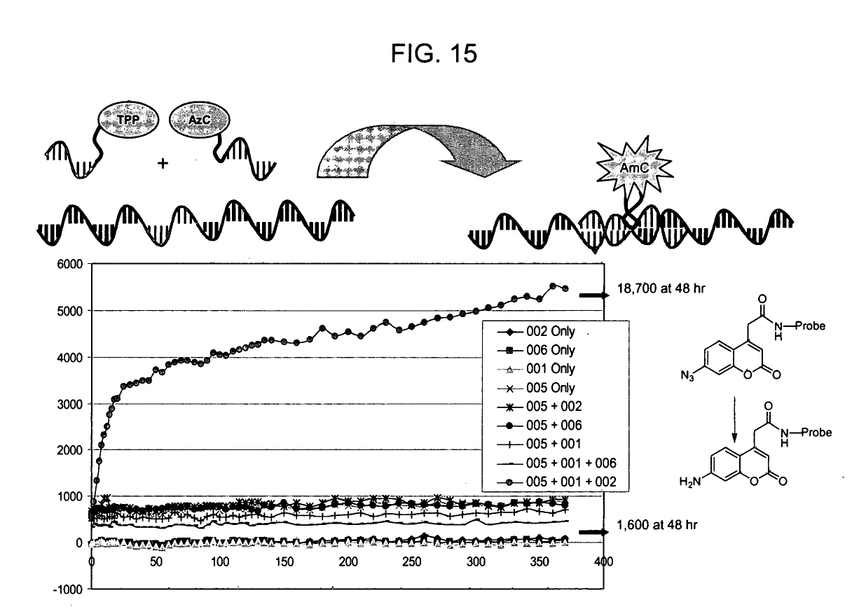

Results of additional experiments involving ternary complexes are shown in FIG. 15. In these experiments Oligo1 is tested for its ability to bring together by hybridization two perfectly complementary oligonucleotides (Oligo5-AzC and Oligo-2TPP) versus its ability to bring together one perfectly complementary oligonucleotide (Oligo5-AzC) and one partially-complementary oligonucleotide (Oligo6-TPP). Oligo1 and Oligo5-AzC were at 200 nM final concentration, whereas Oligo2-TPP and Oligo6-TPP were employed at 400 nM final concentration. In FIG. 15, 001 denotes Oligo1, 002 denotes Oligo2-TPP, 005 denotes Oligo5-AzC, and 006 denotes Oligo6-TPP. The results show that fluorescence is generated only when the combination of fully complementary oligonucleotides is present (Oligo1, Oligo5-AzC and Oligo2-TPP).

### Example 2

### Gene Painting

Gene Painting is a method of sequence detection based upon developing signal at multiple sites within a target. The multiple sites typically lie within a gene sequence that one wishes to show the presence, absence or the quantity of. Within a relatively long sequence, for example a 5,000 base sequence, one can target smaller sequences, typically 40-50 bases, which are unique to that sequence. These are targeted by pairs of oligonucleotide probes, each typically 10-20 bases long. If the probes averaged about 12 bases in length, about 400 pairs of probes can “paint” a 5,000 base long sequence. Each of these probe pairs is a reactive pair (via nucleic acid template chemistry, as described in FIG. 1) and produces a fluorophore from prefluorophore precursors. The total fluorescence generated is the sum of the generation of all 400 fluorophores. To detect, for example, a 5,000 base-long unique gene sequence in a sample of corn genomic DNA simply requires preparation of a sample of corn DNA and its addition to a mixture of 400 oligonucleotide detection probes at a suitable ionic strength, temperature, and formamide concentration. The total fluorescence generated is expected to be proportional to the amount of this gene sequence in the corn DNA. The calculated detection levels based upon the known sensitivity of commercial fluorescence instruments is within the range calculated for the expected fluorescence yield of the nucleic acid templated chemistry-based gene painting technique.

### Example of Assay Design

One exemplary application of the invention is to detect a copy of a transgenic gene in a genetically engineered plant such as corn. The target gene may be, for example, resistance to a herbicide. The gene could be present in a single copy or multiple copies per genome. A typical application is to determine if a particular batch of corn contained this gene or not, and to quantitate the number of average gene copies per genome.

An example of an assay for this gene according to the present invention first involves isolation of circa 100 μg or more of total corn DNA by homogenizing the corn in a blender. The corn DNA can be isolated using any one of a number of kits for extraction and purification of plant DNA. The DNA is sheared to a small average size by, for example, sending it through a hyperdermic needle to render it easier to denature into single strands. The DNA then is heated briefly to 100° C. and quickly cooled to render it single-stranded. A reaction mixture is then added which contains 400 pairs of oligonucleotide probes, each specific for a DNA sequence in the target gene, and each pair containing the two DPC-reactive prefluorophores. Upon incubation, typically at a mildly elevated temperature (37° C.) the fluorescence generated is measured in a fluorescence microplate reader. The fluorescence generated is calibrated using reference samples of corn DNA with known quantities of the target gene. The expected amount of fluorophore generated in this example is about 30 femtomoles, which is well within the detection limits of commercially available microplate readers.

### Example 3

### Oligonucleotide Hybridization, Concentration and Melting Temperatures

A model system was prepared which included two twenty-mer oligonucleotides with a ten-base complementary region and ten-base single stranded spacer arms, further linked to a six carbon spacer arm. These oligos were synthesized both with and without a 5′-biotin (with a 6-carbon spacer arm). As shown below, the complementary region is underlined. A third oligo was identical to the (−) strand oligo but with 4 base mismatches (italicized) to the (+) strand.

| | | |
|---|---|---|
| Oligo 26 | | |
| (+) strand | 5′ CTTCGGCCCAGATATCGT | (SEQ. ID. NO. 24) |
| Oligo 27 | | |
| (−) strand | 3′ GTCTATAGCATCGACATC | (SEQ. ID. NO. 25) |
| Oligo 28 | | |
| (−) mis- | 3′ TACTATAGTGTCGACATC | (SEQ. ID. NO. 26) |
| match | | |

Melting curves of the 10-base pair oligonucleotide pair (oligo 26+oligo 27) were examined by measuring fluorescence of SYBR dye binding to double stranded DNA in a Bio-Rad iCycler (Lipsky, et al., Clinical Chemistry 47[4], 635-44. 2001.) The binding curves are presented as the first derivative of the slope of the melting curve, such that a maximum value represents a point of inflection in the curve (a Tₘ, or in a mixed population of double stranded sites, a “local” Tₘ). Binding curves can be obtained up to at least 70° C. as avidin retains biotin binding activity up to this temperature and beyond.

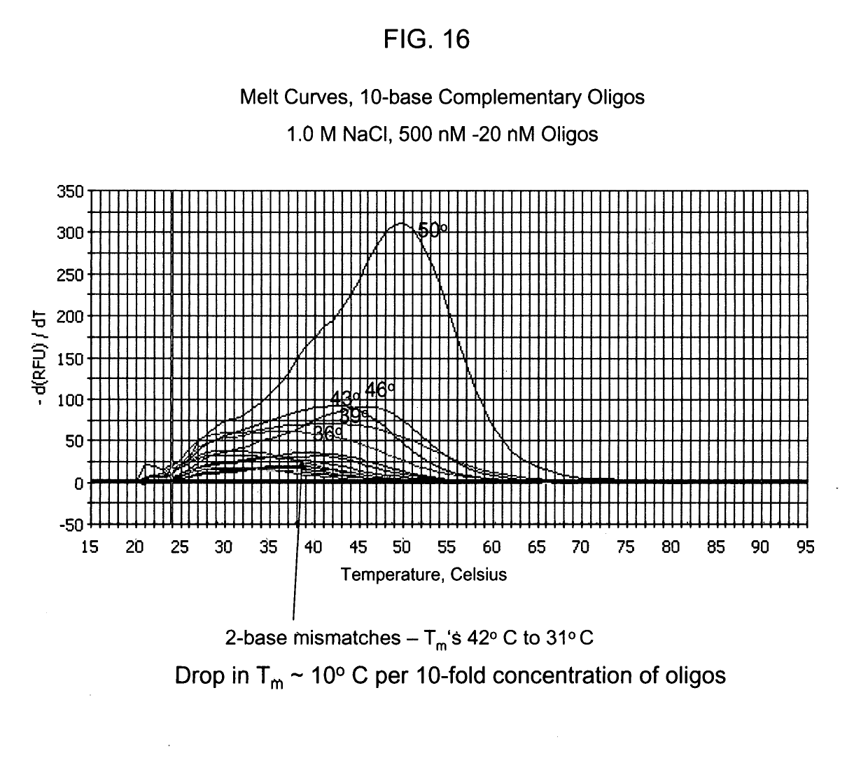

To check the dependence of this particular pair of oligonucleotides upon concentration, melting curves were generated for the oligonucleotide pair varied over the range from 500 to 20 nM (FIG. 16). (See, e.g., Lipsky, et al., Clinical Chemistry 47[4], 635-44. 2001). The observed Tₘ dropped at the rate of about 10° C. per each ten-fold reduction in concentration (where RFU indicates relative fluorescence units) of the oligonucleotide pair, similar to prediction in the graph of FIG. 16. The melting curves were essentially identical for biotinylated and non biotinylated oligonucleotide pairs. The four base mismatched pair showed essentially no double stranded structure.

To test whether binding the (+) and (−) strands to a protein target would cause an increase in Tₘ, the biotinylated version of these oligonucleotides were incubated in the presence of avidin. Avidin contains 4 equivalent binding sites, which are spaced relatively close together and bind to biotin very tightly (Kₐ˜<10⁻¹⁵ M) and non-cooperatively.

Presented with equal molar concentrations of oligonucleotides #26 and #27 in biotinylated form, it would be expected that about half of the biotin binding sites are occupied by complementary pairs of oligonucleotides, and about half with the same oligonucleotide (non-complementary pairs). The prediction is that one would observe two melting curve peaks in the presence of avidin. One peak would be the result of any pairs of oligonucleotides which were either not bound to avidin (free in solution) or which had only one partner of the two bound to avidin, which should not exhibit a proximity effect upon Tₘ. A second peak of significantly, higher Tₘ would represent a pair of biotinylated oligos both bound to avidin, which should exhibit a proximity effect.

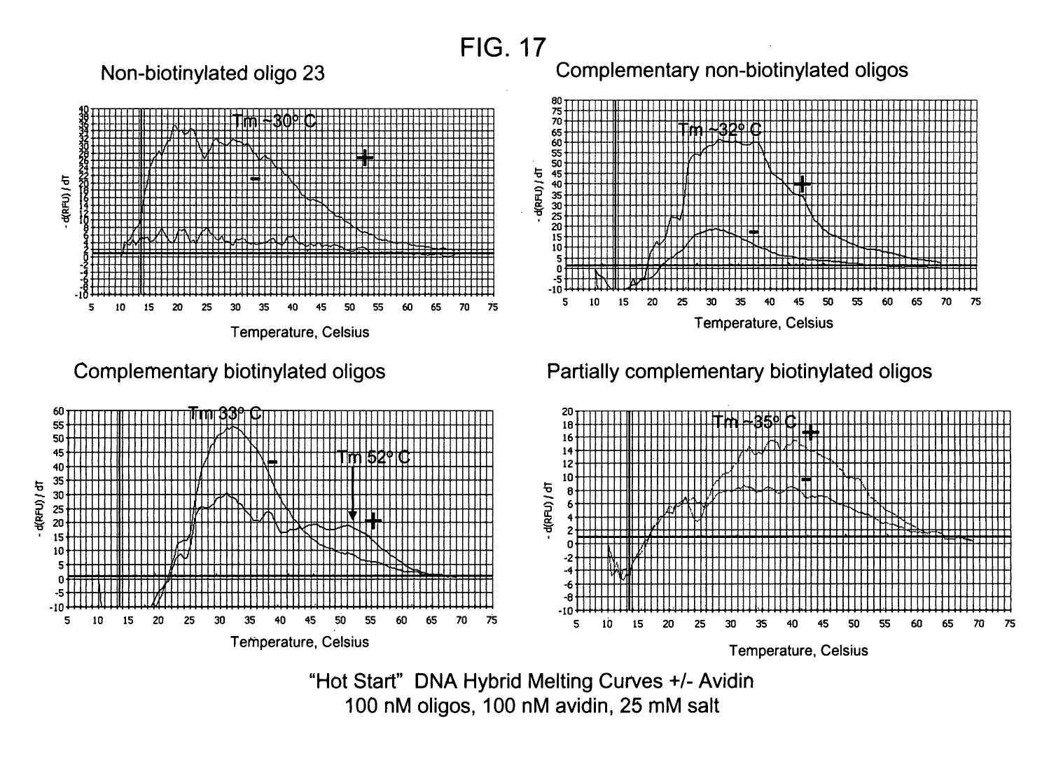

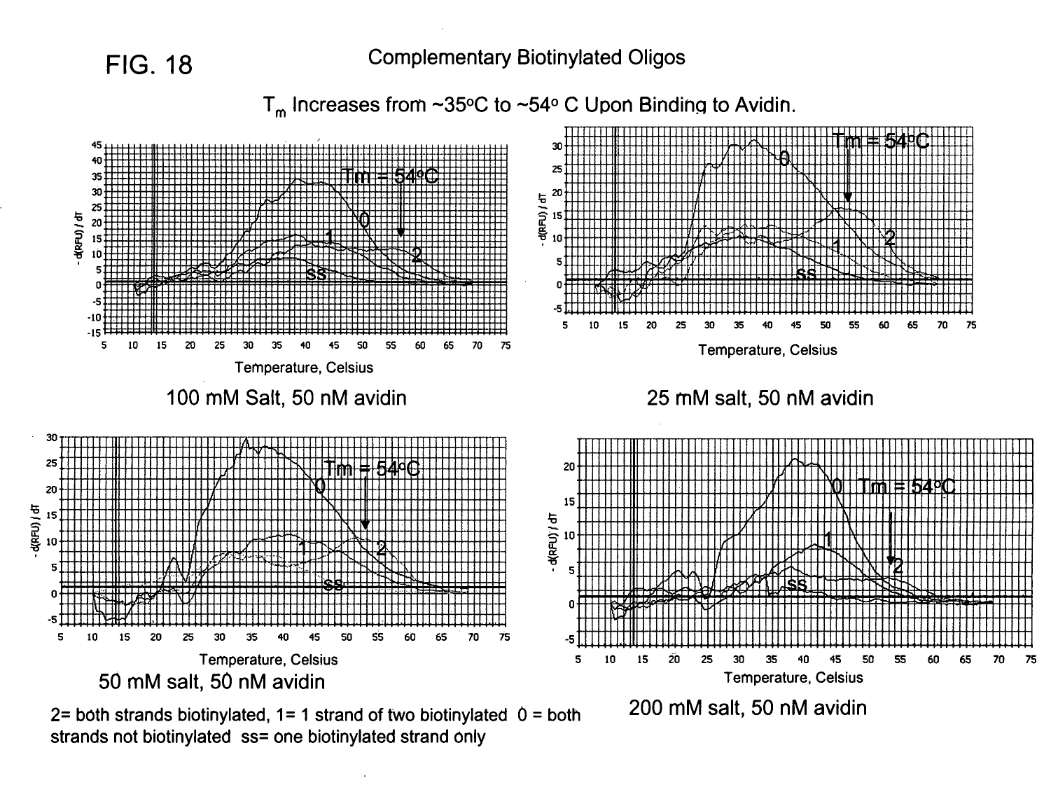

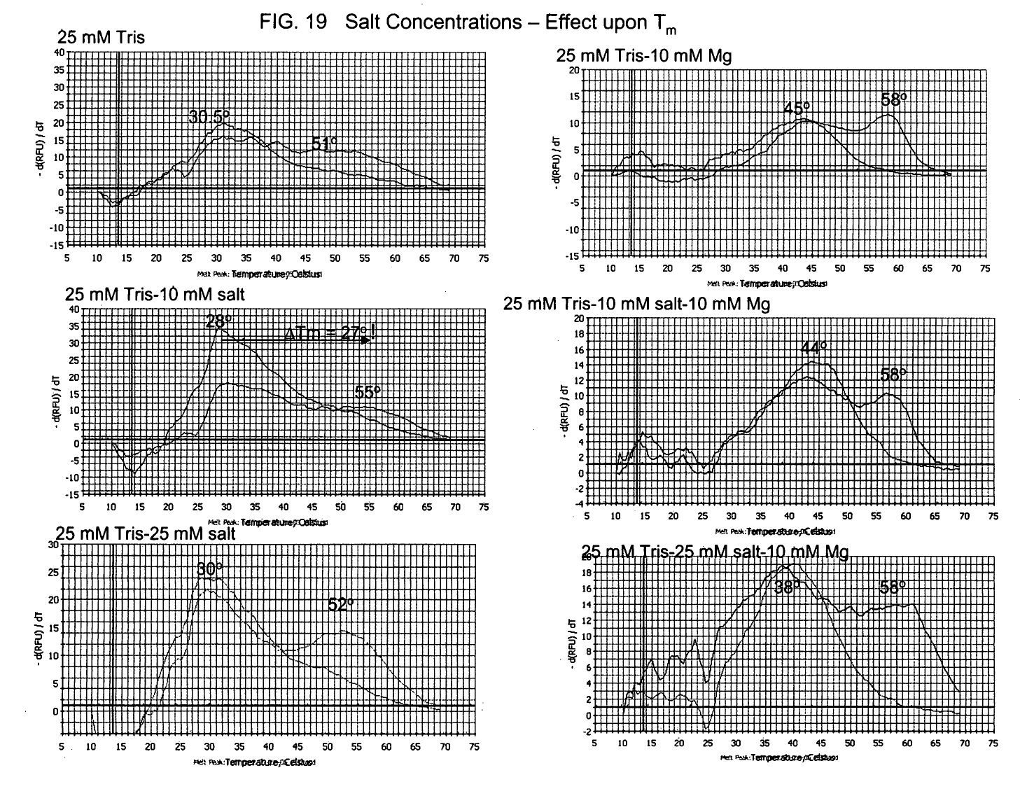

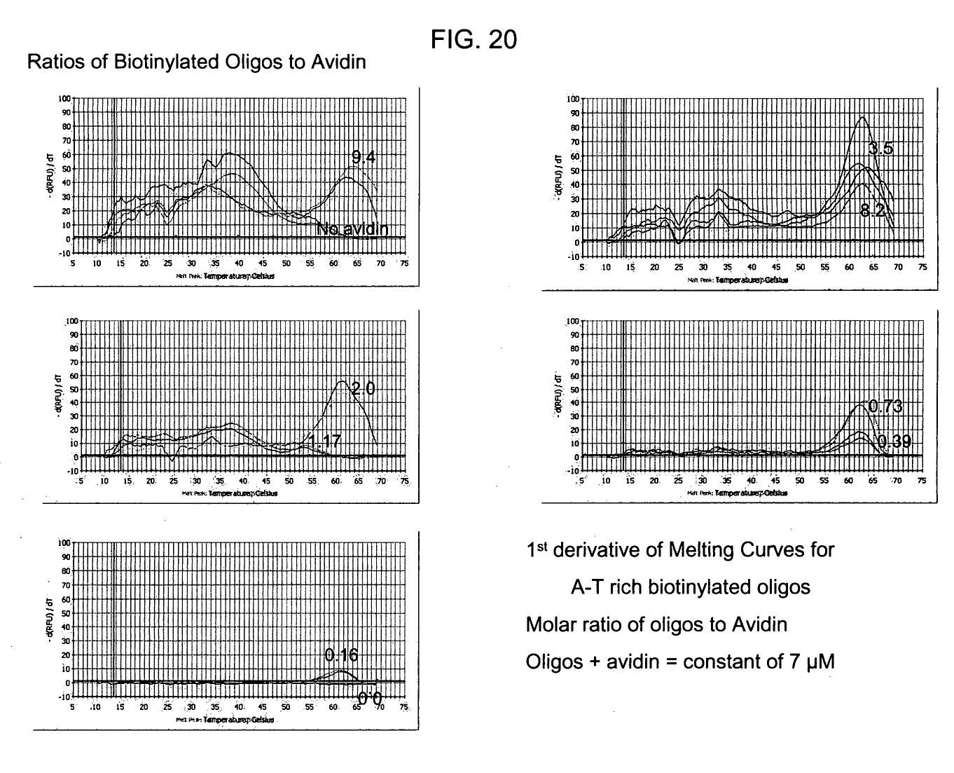

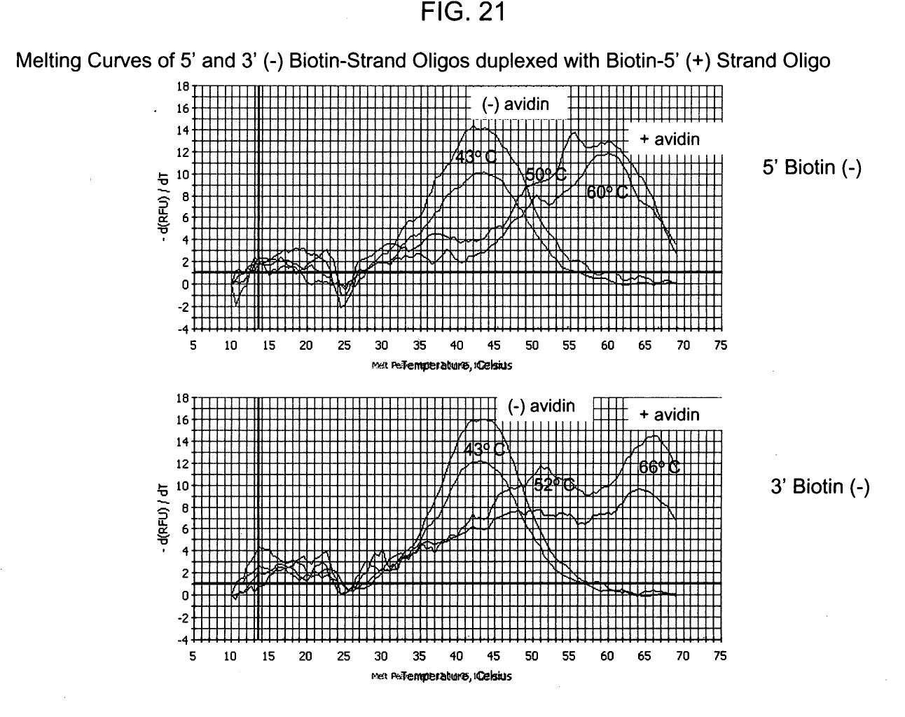

Such an experiment was conducted as shown in FIG. 17. The oligonucleotides were added to a solution in the presence or absence of avidin held at 60° C., a so-called hot start. In a “hot start,” the oligonucleotides bind to the biotin binding sites at a temperature well above their Tₘ in solution, assuring that they are single stranded. The solution was then ramped down to 10° C. and a melting curve analysis performed ascending to 70° C. As shown in FIG. 17, the melting curves of non-biotinylated oligo pair in the presence or absence of avidin showed a Tₘ of 30-32° C. (where RFU indicates relative fluorescence units). In the presence of avidin, however, two well separated Tₘ peaks were generated with Tₘ values of 33° C. and 52° C. The elevated temperature peak (Tₘ raised almost 20° C.) was observed only in the presence of two complementary biotinylated oligonucleotides in the presence of avidin. The difference in Tₘ+/−biotin tended to be greatest at lower salt concentrations (FIG. 18) and slightly higher in the presence of 10 mM magnesium chloride (FIG. 19) (where RFU indicates relative fluorescence units). The optimal molar ratio of biotinylated oligonucleotides to avidin was found to be about 3.5:1, (with total concentration of oligos+avidin=0.7 μM) consistent with avidin possessing four equivalent binding sites (FIG. 20) (RFU indicates relative fluorescence units). This is important because it substantiates that the requirement that the oligonucleotides bind to the same molecule of avidin for the Tₘ effect to occur. The substitution of a 3′ biotinylated (−) strand oligo for a 5′ biotinylated strand oligonucleotide showed little difference in Tₘ values (FIG. 21) (RFU indicates relative fluorescence units) with previous results in which both oligonucleotides were 5′ biotinylated.

Results were essentially identical if the experiment was conducted by adding equimolar amounts of both the oligonucleotides at room temperature, ramping to 60° C., and then obtaining the melting curves. In this method (as well as the hot start method) suitable melting curves can be generated by adding an excess molar of each oligo relative to avidin if desired. (Large excesses of pairs of oligos increases the size of the low Tₘ peak, however, as predicted.) This was not detrimental in forming high Tₘ hybrid DNA since the pairs of oligos competed equally for biotin binding sites as long as they were added together in equal molar amounts. If oligos were added one at a time, it was important to add about a 2:1 molar ratio of the first oligo to avidin followed by a 2:1 ratio of the second oligo. With sequential addition, adding an excess molar amount of either oligo relative to avidin occupies all the binding sites of the avidin with the first oligo and prevents occupying adjacent sites with the second, complementary oligo and exhibiting the elevated Tₘ effect. These observations are consistent with the mechanism being binding of adjacent pairs of complementary oligos to two adjacent biotin binding sites to obtain hybrids exhibiting the elevated Tₘ peaks.

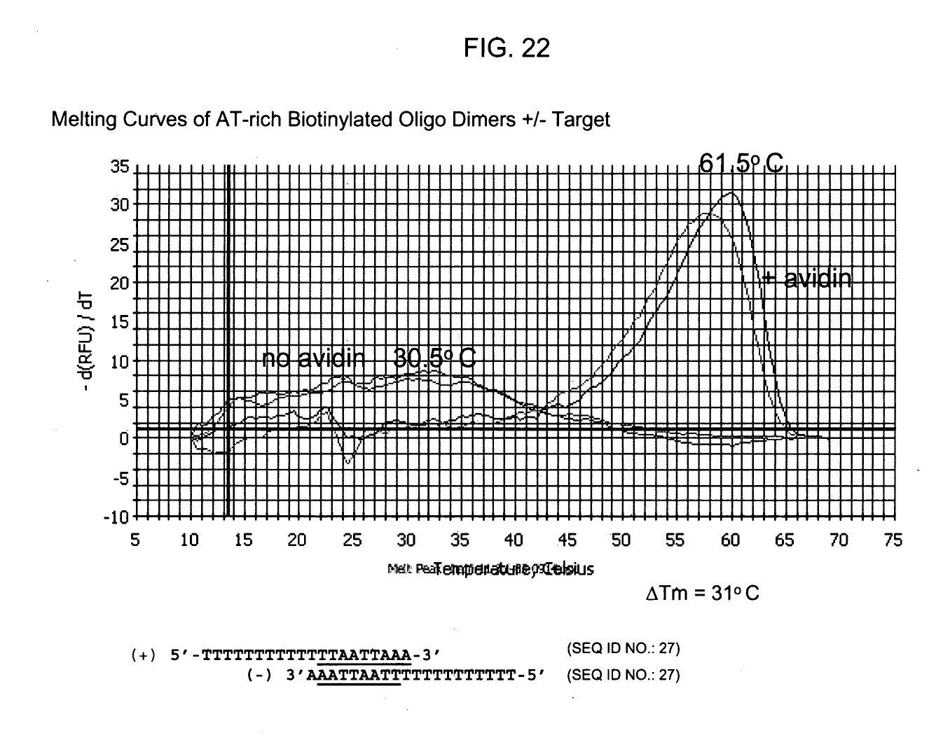

Experiments were also conducted with a 10-base self-complementary oligonucleotide which was composed entirely of A and T. (Oligo 31: 5′-biotin-spacer arm-TTTTTTTTTTTTTAATTAAA) (SEQ. ID. NO. 27). Because this oligonucleotide was homogeneous in base composition and composed entirely of AT, it melted at a lower Tₘ than the above-described model system and produced a fairly sharp melting curve. In the presence of avidin, its Tₘ was increased from 30.5° C. to 61.5° C. (FIG. 22) (where RFU indicates relative fluorescence units). Since this oligonucleotide was self-complementary, all binding events lead to complementary strands, rather than only ½ of the events. Thus, only a single peak of increased Tₘ was observed.

These experiments were repeated using anti-biotin antibody as a target rather than avidin. Anti-biotin antibody contains two biotin binding sites located near the ends of the Fab portion of the antibody, but the binding sites are much further apart than the biotin binding sites on avidin.

### Example 4

### Detection of Protein Targets—Aptamers as Target Binders

Here, an exemplary system was designed to utilize nucleic acid-templated azidocoumarin (AzC)-triphenylphosphine (TPP) chemistry to detect a protein target upon aptamer binding and annealing of the two complementary DNA probes.

Materials

Human PDGF-BB and PDGF-AA was obtained from R&D Systems (220-BB and 220-AA, respectively). Anti-human PDGF-B Subunit monoclonal antibody was obtained from R&D Systems (MAB2201). Buffers included Tris/Mg buffer, at 50 mM Tris/HCl, pH 8.0-10 mM MgCl₂. Oligonucleotides used were as follows:

Oligonucleotide Sequences Used in this Example

| Oligo #/ | | | | |
|---|---|---|---|---|
| (SEQ. | | 5′- | 3′- | |
| ID #) | Sequence (5′ to 3′) | Mod'f. | Mod'f. | Description |
| 201 | CAGGCTACGGCACGTAGAGCATCACCATGATCCTGC | TPP | none | DPC-aptamer |
| (28) | CCCCCCCCCATATTTAAGC | | | probe |
| 202 | GCTTAAATATCCCCCCCCCCCAGGCTACGGCACGTA | none | AZC | DPC-aptamer |
| (29) | GAGCATCACCATGATCCTG | | | probe |
| 203 | GTGGGAATGGTGCCCCCCCCCCCAGGCTACGGCAC | none | AZC | DPC-aptamer |
| (30) | GTAGAGCATCACCATGATCCTG | | | probe-mismatch |
| 204 | GTGGTAGTTGGAGTCGTGGCGTAGGCAAGA | none | none | target |
| (31) | | | | |
| 205 | GTGGTAGTTGGAGTCACACGTGGCGTAGGCAAGA | none | none | target |
| (32) | | | | |
| 206 | GTGGTAGTTGGAGCTCACACCACACGTGGCGTAGG | none | none | target |
| (33) | CAAGA | | | |
| 207 | GTGGTAGTTGGAGTCACACACACCACACACAGTGG | none | none | target |
| (34) | CGTAGGCAAGA | | | |
| 208 | GTGGTAGTTGGAGCTCACACCACACCAACCACACC | none | none | target |
| (35) | ACACCACACACACCACACGTGGCGTAGGCAAGA | | | |
| 209 | GTGTGGTGTGGTGTGGTGTG | none | none | splint |
| (36) | | | | K-ras target |
| 210 | GTGGCGTAGGCAAGAGTGGTAGTTGGAGCT | none | none | outward facing |
| (37) | | | | |
| 211 | GTGGGAATGGTG | none | TPP | TPP probe |
| (38) | | | | |
| 212 | AGATCCCACTAGCAC | TPP | none | TPP probe |
| (39) | | | | |
| 213 | AGCTCCAACTACCAC | TPP | none | TPP “mismatch” |
| (40) | | | | |
| 214 | TCTTGCCTACGCCAC | none | AZC | AZC probe |
| (41) | | | | |
| 215 | CAGGCTACGGCACGTAGAGCATCACCATGATCCTG | none | none | aptamer |
| (42) | | | | |

Methods

DPC Reaction conditions. Except as noted, each 100 microliter reaction contained, in a total volume of 100 μl, 1×Tris/Mg buffer, 40 picomoles of TPP and AzC reaction probes, 40 picomoles of target oligonucleotide or of target protein, and typically 25-30% v/v of formamide, Samples were incubated at 25° C. in a Wallac Victor 1420 spectrophotometer and the increase in fluorescence monitored with excitation at 355 nm and emission at 460 nm.

Results: Detection of PDGF-BB by Aptamer-DPC Probes

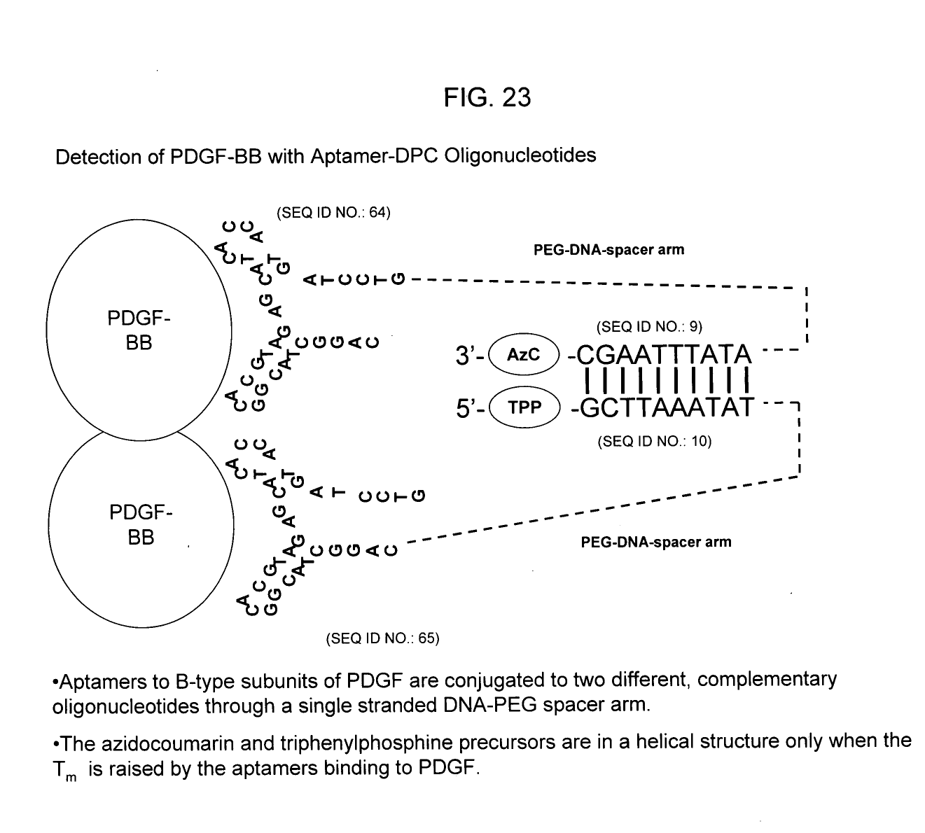

As illustrated in FIG. 23, an aptamer sequence directed against platelet-derived growth factor (PDGF) B-subunits was selected (Fang, et al., Chem. BioChem. 4, 829-34. 2003). This belongs to a family of aptamers with strong affinity for PDGF B subunit (10⁻⁹ M), and about ten-fold reduced affinity for PDGF A subunit. (Green, et al., Biochemistry 35, 14413-24. 1996) The probe sequences were synthesized, each containing a complementary 10-mer DNA sequence, a C₁₀ spacer sequence, and the same 35-mer aptamer sequence. (Oligos #201, #202). Each sequence contained a 5′-TPP or 3′-AZC group with the aptamer linked 3′ or 5′, respectively. A second AzC probe, oligo #203, was the same as oligo #202 except that its annealing sequence was entirely mismatched to the TPP oligo (#201).

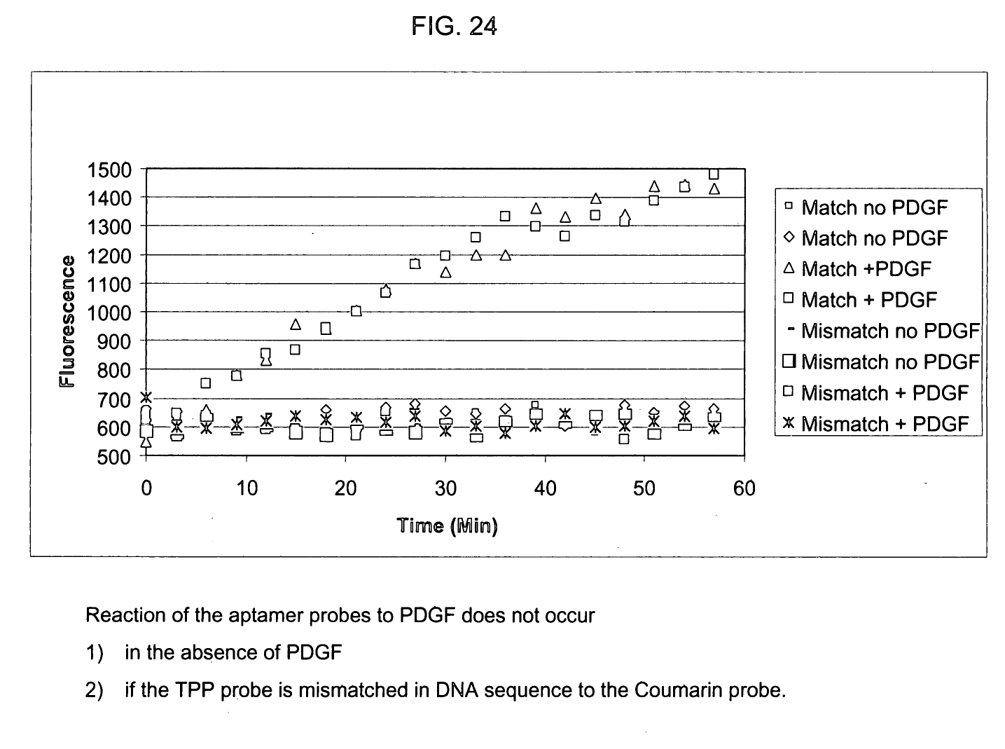

As shown in FIG. 24, in the presence of 30% (volume) formamide, the reaction of the TPP and AzC probes with each other was entirely dependent upon the presence of PDGF-BB and complementary DNA sequences on the probes. The reaction failed in the absence of either probe.

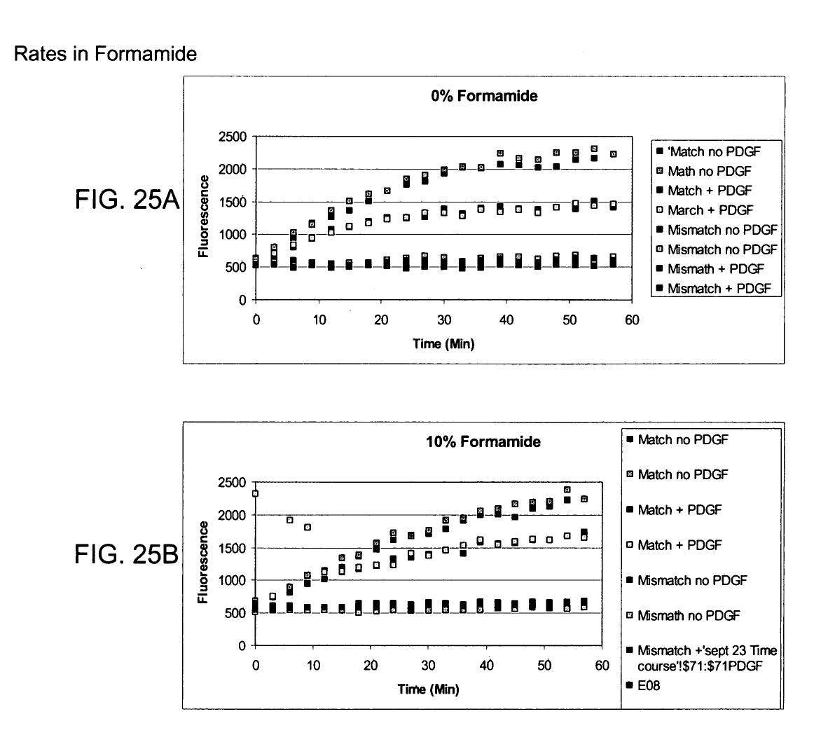

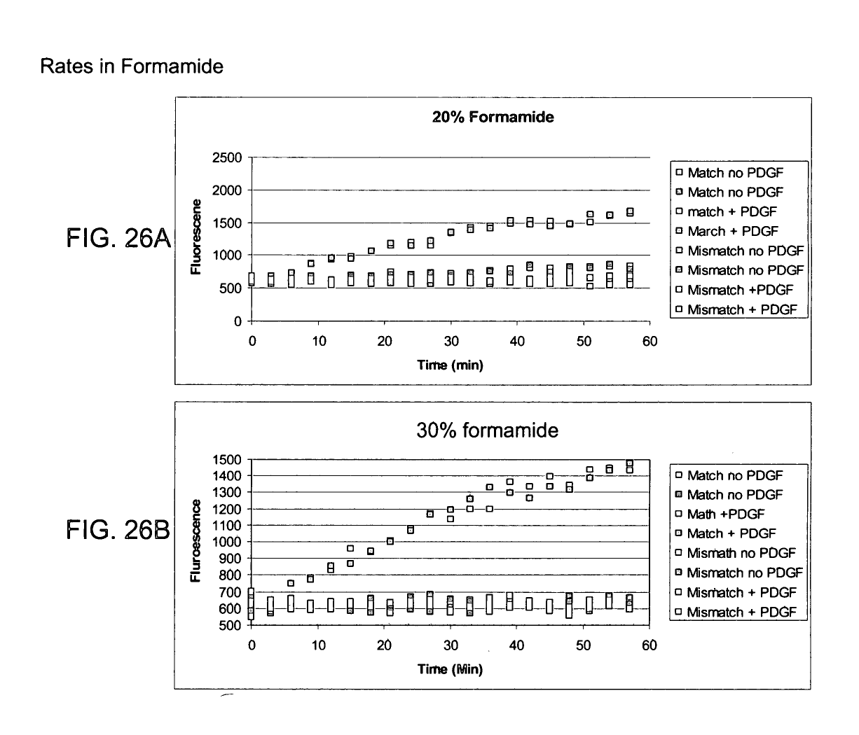

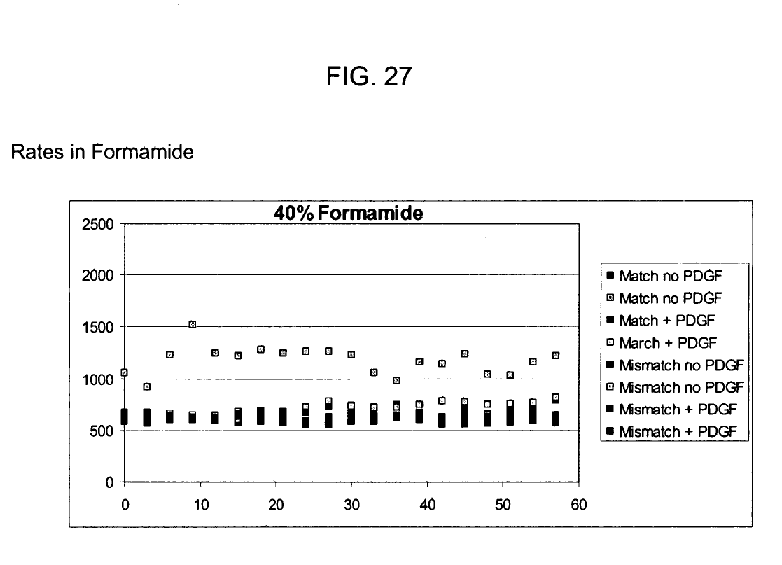

The DNA-dependence of the reaction was critically dependent upon the melting temperature of the DNA relative to the assay temperature. In the presence of 0% formamide (with the calculated and observed Tₘ>T_{assay} the reaction took place in the presence or absence of the target protein PDGF-BB (FIG. 25A). In fact, under these conditions, addition of PDGF-BB did not increase, but reduced the reaction rate by about 50%. In 10% formamide, PDGF-BB was less inhibitory (FIG. 25B). In 20% formamide (FIG. 26A), the situation was completely reversed—the reaction rate was now weak except in the presence of PDGF-BB. In 30% formamide (FIG. 26B) the reaction was completely dependent upon the presence of PDGF-BB. In 40% formamide, the reaction was very slow with any set of reactants (FIG. 27). In all cases, the mismatched probes produced little or no reaction.

DNA melting experiments with the complementary sequences, monitored with SYBR Green had indicated a Tₘ of the sequence of about 30° C. in the Tris/Mg buffer in the absence of formamide, and about 7° C. lower for every 10% increase in formamide. Tₘ in the optimal formamide concentration for the detection assay, 30%, was 10° C.

In 0% formamide, the oligonucleotides can form at least a partial duplex even in the absence of PDGF-BB (Tₘ slightly higher than T_{assay}). The DNA target-dependence of the reactions in 20% and 30% formamide is explained by the assay being conducted at a temperature greater than the Tₘ in the absence of protein target. No reaction occurs unless the Tₘ of the complex is increased by the binding of the two probes to the PDGF-BB target. At 40% formamide, the reaction doesn't occur with any set of reactions. The likely explanation is that either the Tₘ had been reduced so low that binding to PDGF-BB could not raise it above T_{assay}, or that formamide had inhibited PDGF-BB binding to the aptamers. A more complex situation is the observed inhibition of reaction rate upon addition of PDGF-BB in the absence of formamide. Since half of the duplexes formed by PDGF-BB are non-productive (50% will be homoduplexes) the reduction in rate is likely due to PDGF-BB binding preventing these homoduplexes from disassociating and then reassociating in solution with complementary pairs to form heteroduplexes. This situation should not occur using pairs of probes specifically directed against different binding sites in a heterodimeric target.

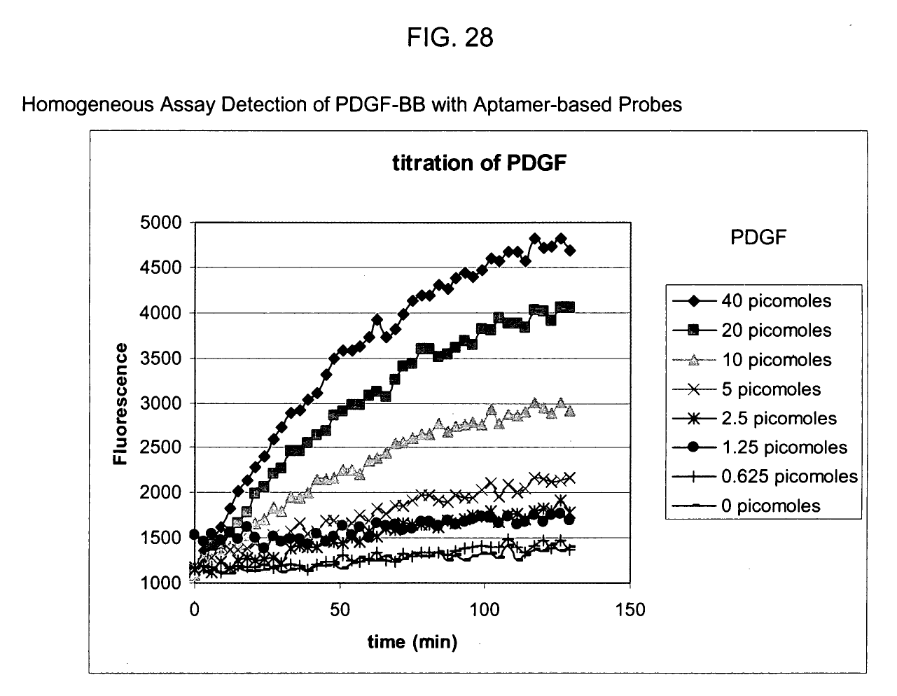

The sensitivity of the assay (FIG. 28) was calculated by measuring reaction rates generated from a dilution series of PDGF-BB concentrations. The minimum detection level on the Wallac instrument was estimated at 0.8 picomoles in a 100 microliter assay volume, based upon the calculated value of three times the standard deviation of the background noise of the assay.

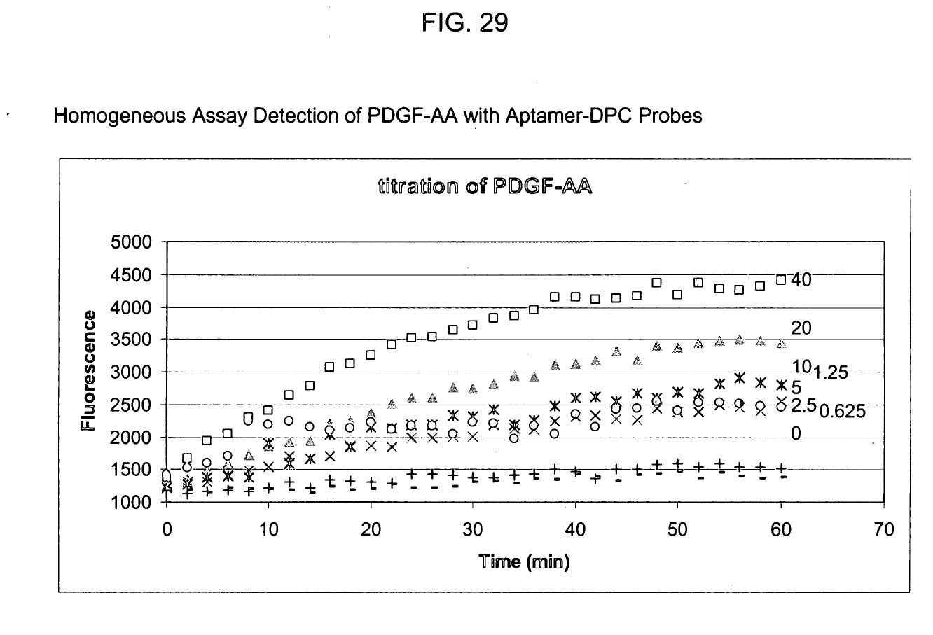

The assay sensitivity was also determined using PDGF-AA as a target. The aptamer monomer is expected to have an affinity for PDGF-AA about ten times weaker than for PDGF-BB. However, since the assay involves forming a complex of two aptamer-dimers to either type of PDGF, the avidity of binding of the dimer is expected to be tighter than the affinity of the monomer, and its affinity should be substantially tighter (lower Kᵢ) than the concentrations tested of the target PDGFs (down to about 1 nanomolar). As shown in FIG. 29, the reaction rates of the aptamer DPC probes to PDGF-AA at low or high concentrations (0, 1.25, 2.5, 5, 10, 20, and 40 pmole of PDGF-AA) were not substantially different than the reaction rates with PDGF-BB. This is consistent with the model of an aptamer pair binding as a dimer and exhibiting increased avidity.

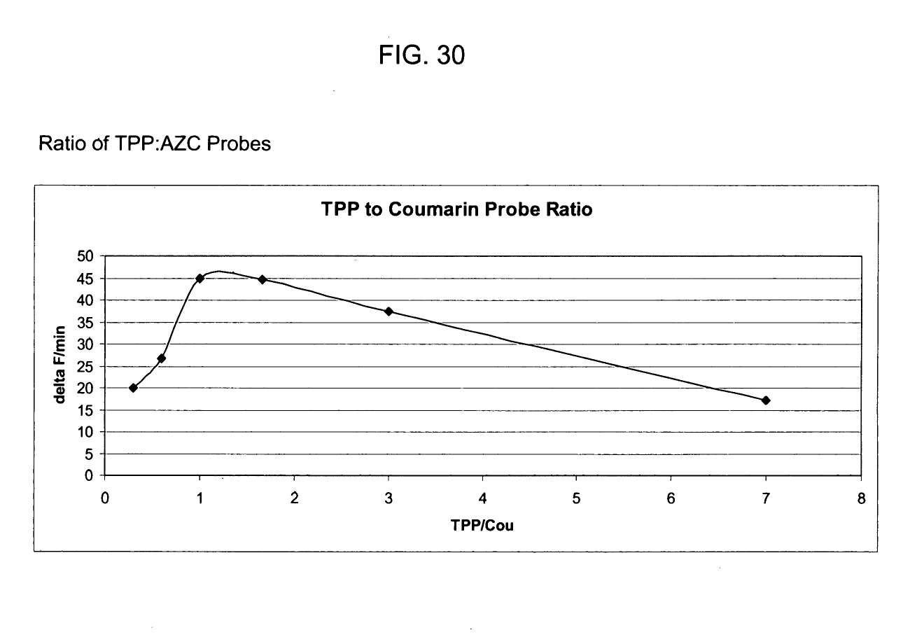

Ratios of TPP to AzC Probes. To confirm the model of the reaction mechanism (FIG. 4, the optimal ratio of TPP to AzC probes would be expected to be 1:1), FIG. 30 was an experiment in which the total amount of the two probes was kept constant, at 800 nMoles probes/reaction, while the ratio of the two probes was varied. The ratio producing the highest reaction rate was approximately 1:1, consistent with the expected mechanism.

Thus, in this model system fluorescence was not generated unless the aptamers bound and the complementary sequences in the two probes annealed to each other.

### Example 5

### Zip-Coded Architecture for Nucleic Acid-Templated Chemistry Based-Biodetection with Aptamer Binders

FIG. 10 illustrates in more detail an exemplary zip-code architect. The TPP pair contained, first, a PDGF-aptamer on the 5′-end, a C18 polyethylene-glycol based spacer, and an 18-mer zip code sequence. The TPP reporter sequence contained a complementary anti-zip code sequence on its 3′ terminus, a C18 PEG spacer, and a ten base pair reporter sequence terminating in a 5′ TPP group. The pair of oligonucleotides comprising the AzC detection probe contained a 3′-aptamer linked through a C18 PEG spacer to a separate zip code, and a detection oligonucleotide linked to a 5′ anti-zip code, a C18 PEG spacer, and a reporter oligonucleotide (complementary to the TPP oligonucleotide) terminating in a 3′ AzC group.

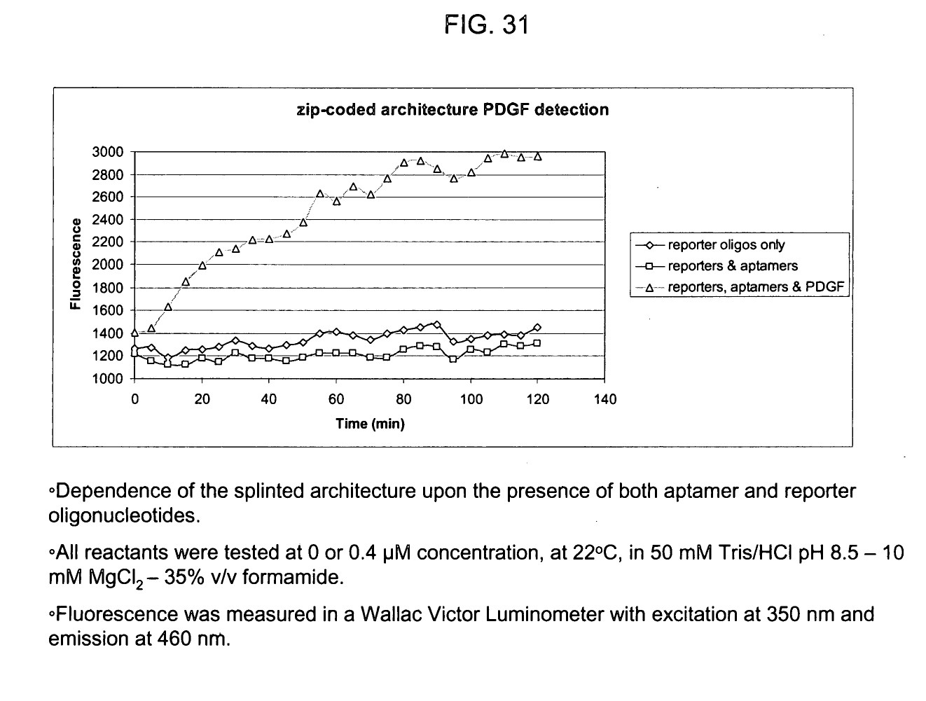

The reaction, in 35% formamide at 22° C., was dependent upon the presence of both of the reporter oligonucleotides, both of the aptamer oligonucleotides, and the target, PDGF-BB (FIG. 31). At 22° C. in the absence of formamide, the reaction proceeded independently of the presence of PDGF. This is consistent with the behavior of the above-described “one-piece” architech, and reflects that the mechanism of fluorescence generation in 35% formamide is dependent the increased thermal stability of the reporter sequence duplex in formamide upon addition of PDGF. In the absence of formamide at 22° C., the reporter oligonucleotide duplex is stable both in the presence and absence of PDGF.

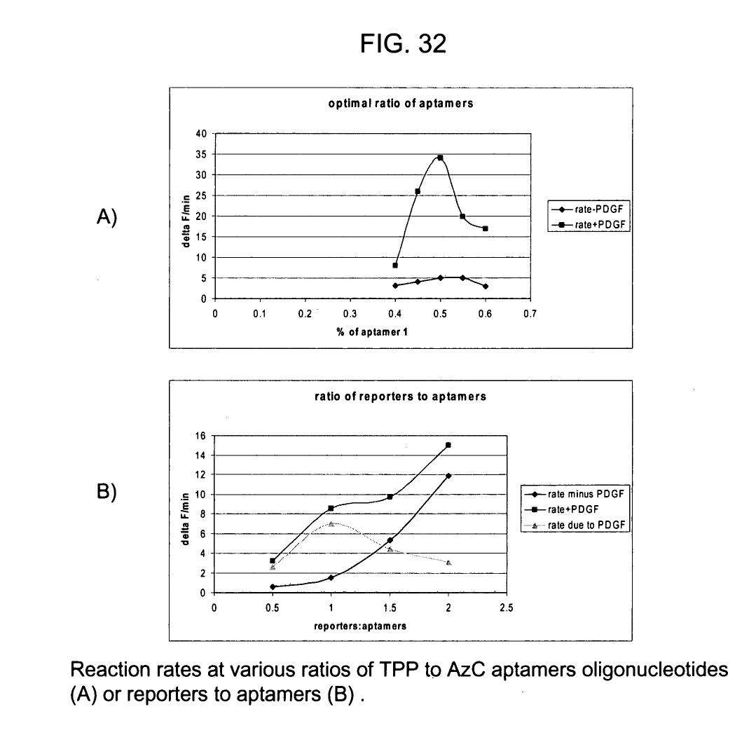

Confirmation of the correctness of the model was obtained with experiments varying the ratio of the TPP and AzC aptamer oligos (FIG. 32). These experiments indicated that the optimal ratio of the aptamer oligos was the expected 1:1 ratio (i.e. 50% TPP oligo with a total concentration of PDGF and aptamer oligos of 0.4 μM). The optimal ratio of total reporter oligonucleotides to total aptamer oligos was also 1:1. No PDGF-dependent reaction occurred in the complete absence of either one of the reporter or aptamer oligonucleotides. At higher than stoicheometric concentrations of reporter oligonucleotides, the PDGF-independent signal increased (background) but the PDGF-dependent signal remained about constant. Both of these observations are consistent with the model that the complex is assembled in the ratio of 1:1:1 for each of the aptamer oligos, each of the reporter oligos, and PDGF.

These experiments indicate that the complex can self-assemble in solution, such that each zip code and its anti-zip code anneal to each other with minimal interference with the aptamer sequence or the reporter sequences.

Experiments were also conducted to determine if the order of addition, and thus assembly of the aptamer and reporter probes, was of any importance. Slightly slower reaction rates were obtained if the aptamer oligonucleotides were first incubated with PDGF before adding the reporter oligonucleotides, compared with adding all probes together as a mixture. Somewhat greater reaction rates were obtained if each pair of aptamer oligonucleotides and reporter oligonucleotides was first incubated and allowed to assemble with each other before the two sets were mixed together and incubated with PDGF. The reason for this may be that there is some steric hindrance to zip code-anti zip code annealing to aptamer probe if the aptamer probe is already bound to target.

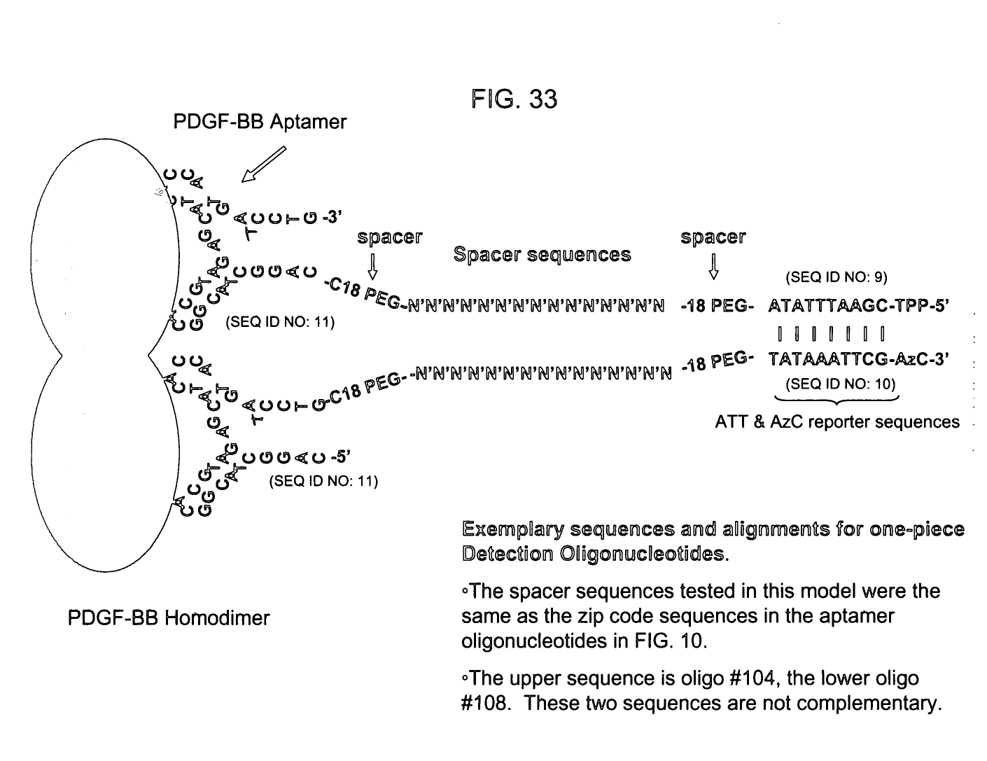

As a control, a set of one-piece TPP and AzC probes was compared which contained only the zip code sequences and no zip code-anti zip code sequences (FIG. 33). The reaction rates of this one-piece system were similar to that of the two-piece system, except that the rate enhancement due to the addition of PDGF was typically slightly better than that of the two-piece system.

The sequence of the aptamer-containing TPP and AzC probes was also systematically varied to determine any constraints on the design. The aptamer-containing TPP and AzC oligos were synthesized, both having the same sequences as described in FIG. 10 but with the following changes: (1) omission of the C18-PEG spacer. (Oligos 119 & 122); (2) replacement of the C18-PEG spacer with the sequence C₁₀. (oligos 120 & 123); (3) replacement of the C18-PEG spacer with the sequence C₂₀. (oligos 121 & 124); (4) Omission of the C18-PEG spacer and omitting 3 3′-bases in the zip code region (reduction to 15 bases in length). (oligos 127 & 129); and (5) omission of the C18-PEG spacer and omitting 6 3′-bases in the zip code region (reduction to 12 bases in length). (oligos 128 & 130).

Oligonucleotides Used in this Example Included:

| | | |
|---|---|---|
| Oligo#/ | Sequence (5′-3′) | Modification |

(SEQ. ID NO. 43)

| | | |
|---|---|---|
| 106 | GGACTCGAGCACCAATAC-X-TATAAATTCG-AZC | X = C18 PEG; |
| | | AZC = 3′-AzC. |

(SEQ. ID NO. 44)

| | | |
|---|---|---|
| 109 | CGAATTTATA-X-CTGACCATCGATGGCAGC | X = C18 PEG, |
| | | 5′-TPP |

(SEQ. ID NO. 45)

| | | |
|---|---|---|
| 112 | CAGGCTACGGCACGTAGAGCATCACCATGATCCTG-X-GCTGCCATCGATGGTCAG | X = C18 PEG |

(SEQ. ID NO. 46)

| | | |
|---|---|---|
| 113 | GTATTGGTGCTCGAGTCC-X-CAGGCTACGGCACGTAGAGCATCACCATGATCCTG | X = C18 PEG |

(SEQ. ID NO. 47)

| | |
|---|---|
| 119 | GTATTGGTGCTCGAGTCCCAGGCTACGGCACGTAGAGCATCACCATGATCCTG |

(SEQ. ID NO. 48)

| | |
|---|---|
| 120 | GTATTGGTGCTCGAGTCCCCCCCCCCCCCAGGCTACGGCACGTAGAGCATCACCATGATCCTG |

(SEQ. ID NO. 49)

| | |
|---|---|
| 121 | GTATTGGTGCTCGAGTCCCCCCCCCCCCCCCCCCCCCCCAGGCTACGGCACGTAGAGCATCACCATGATCCTG |

(SEQ. ID NO. 50)

| | |
|---|---|
| 122 | CAGGCTACGGCACGTAGAGCATCACCATGATCCTGGCTGCCATCGATGGTCAG |

(SEQ. ID NO. 51)

| | |
|---|---|
| 123 | CAGGCTACGGCACGTAGAGCATCACCATGATCCTGCCCCCCCCCCGCTGCCATCGATGGTCAG |

(SEQ. ID NO. 52)

| | |
|---|---|
| 124 | CAGGCTACGGCACGTAGAGCATCACCATGATCCTGCCCCCCCCCCCCCCCCCCCCGCTGCCATCGATGGTCAG |

(SEQ. ID NO. 53)

| | |
|---|---|
| 127 | CAGGCTACGGCACGTAGAGCATCACCATGATCCTGGCTGCCATCGATGGT |

(SEQ. ID NO. 54)

| | |
|---|---|
| 128 | CAGGCTACGGCACGTAGAGCATCACCATGATCCTGGCTGCCATCGAT |

(SEQ. ID NO. 55)

| | |
|---|---|
| 129 | TTGGTGCTCGAGTCCCAGGCTACGGCACGTAGAGCATCACCATGATCCTG |

(SEQ. ID NO. 56)

| | |
|---|---|
| 130 | GTGCTCGAGTCCCAGGCTACGGCACGTAGAGCATCACCATGATCCTG |

None of these changes resulted in a significant difference in the performance of the system. Experiments 4) and 5) also resulted in a 3 and 6-base single stranded (not annealed to zip code) structure immediately upstream of the C18 spacer in the reporter oligonucleotides.

The results of these experiments indicate that the aptamer-based PDGF detection system can be assembled separating the binding and DPC functions into two separate oligonucleotides. Through the selection of appropriate zip code sequences, the detection format described in FIG. 9 self-assembled into pairs of annealed oligonucleotides which will function similarly to oligonucleotides synthesized in a single piece. The reporter and aptamer oligonucleotides may be separately assembled prior to introduction of target, or all species may be added together in almost any order. This process may be extended to the solution-phase assembly of more than one pair of annealed detection oligos, for example, to detect multiple targets. Detection of multiple targets may require using different reporter oligonucleotides which generate separately discernable signals (for example, different wavelengths of emitted light).

These results indicate that a zip-coded reporting approach can be effectively designed, for example, using aptamer-containing oligonucleotides.

While the results with the aptamer system indicate that a stable complex between binding and reporter sequences can be formed simply by annealing the zip code and anti-zip code regions, it should be noted that there are technologies to covalently and irreversibly link the two oligonucleotides together, with a high likelihood of retaining activity of the reporter reactive groups. For example, the oligonucleotides may be incubated in pairs (a binder oligonucleotide and a reactive oligonucleotide for nucleic acid-template chemistry) at a temperature at which the zip codes and anti-zip codes are mostly double stranded, but the rest of the sequences are single-stranded. Adding an intercalating, photoactivatable cross-linker such as Trioxalen, followed by UV irradiation, may irreversibly crosslink the two strands. Similarly, UV irradiation may introduce thymidine dimers between separate strands of annealed sequences. Alternately, a sequence may be introduced complementary to a short target (splice) DNA, abutting 3′ and 5′, which may then be ligated with DNA ligase. The splice oligonucleotide may alternately be composed of RNA, and removed after ligation with RNase H, which hydrolyzes RNA annealed to DNA. This can result in converting the two oligonucleotides into a single piece of single-stranded DNA. These methods can lead to cost-effective production of oligonucleotide reagents in detection kits against specific targets.

Relevant references for this example include Capaldi, et al., Nucleic Acid Res. 28[7], e21. 2000; Castiglioni, et al., Appl. and Exper. Microbio. 2004, 7161-72. 2004; Fang, et al., Chem. BioChem. 4, 829-34. 2003; Gerry, et al., J. Mol. Biol. 292, 251-62. 1999.

### Example 6

### Zip-Coded Architecture for DPC-Based Biodetection—Antibody Binders

In another embodiment, the aptamer sequences are replaced with non-DNA binders such as antibodies. For PDGF and other protein targets, the aptamer sequences are replaced with chemically active groups, such as aldehydes, and reacted with non-DNA binder sequences such as antibodies or receptors to the protein targets (FIG. 34). The optimal design for the binder and reporter oligonucleotides may be achieved with considerations on the size and geometry of the binder and size and geometry of the binding sites of the target. A longer, or shorter spacer arms, for example, may be used to optimally span the distance between binding sites on the target and avoid steric hindrance due to the binders themselves.

Referring to FIG. 34, the zip-coded oligonucleotide designed to hybridize to the TPP reporter molecule was synthesized containing a 5′-amino group. The zip-coded oligonucleotide designed to hybridize to the AzC reporter molecule contained a 3′-amino group. Synthesis of the conjugates between the oligonucleotides and anti-PDGF-BB antibody were performed by SoluLink Biosciences (San Diego, Cailf.).

The SoluLink technology for conjugation of the antibody and oligonucleotides first requires modification of the primary amino groups of the antibody with succinimidyl 2-hydrazinonicotinate acetone hydrazone) to incorporate an acetone hydrazone onto the antibody. The primary amines of the oligonucleotides are separately activated with succinimimdyl 4-formylbenzoate. The two activated molecules are mixed in the desired ratio (typically 6:1) and reacted at a mildly acidic pH to form a stable hydrazone linkage. The details of this chemistry are available at www.SoluLink.com. Two conjugates were prepared: one containing the zip code to anneal to the AzC-containing reporter oligonucleotide, and the other containing the zip code to anneal to the TPP-containing reporter oligonucleotide.

The antibody-oligonucleotide conjugates received from SoluLink were further purified by gel chromatography on a 1.6×60 cm column of Superdex S-200 (Amersham Biosciences) in PBS buffer (0.01 M potassium phosphate, pH 7.4-0.138 M sodium chloride). The main antibody peak, eluting at about 0.6 times the column volume, was collected and a later eluting peak of contaminating non-conjugated oligonucleotide was discarded. The antibody conjugate was concentrated by reversed dialysis with a Pierce (Rockford, Ill.) 30 K molecular weight cut-off Slide-A-Lyzer using Pierce Concentrating Solution. The protein content was determined using the Bio-Rad Micro BCA Reagent Kit and the oligonucleotide content determined using SYBR Gold DNA binding dye (Molecular Probes (Eugene, Oreg.). The conjugates were both determined to contain an average of approximately 3 oligonucleotides per protein molecule.

Recombinant human PDGF-BB (220-BB) and mouse monoclonal anti-PDGF-BB (MAB220) were obtained from R&D Systems (Minneapolis Minn.).

Sequences used in this study included (where AzC indicates azidocoumarin and TPP indicates triphenylphosphine):

| | |
|---|---|
| Name | Sequence (5′-3′) |

| | |
|---|---|
| TPP reporter | TPP-(amino modifier C6)-CGAATTTATA-C18PEG-TCAGCATCGTACCTCAGC |
| | (SEQ ID NO.: 9) (SEQ ID NO.: 58) |
| AzC reporter | GGACTCGAGCACCAATAC-C18 PEG-TATAAATTCG-(amino modifier C7)-AzC |
| | (SEQ ID NO.: 14) (SEQ ID NO.: 10) |
| AzC zip code | TTGGTGCTCGAGTCCCCCCCCCCCCCCCCCCCCCC-(amino modifier C7) |
| | (SEQ ID NO.: 59) |
| TPP zip code | (amino modifier C6)-CCCCCCCCCCCCCCCCCCCCGCTGAGGTACGATGCTGA |
| | (SEQ ID NO.: 60) |

In addition, the 5′ amino modifier C6 was obtained from Glen Research (from Glen Research phosphoramidite 110-1906). The 3′-amino modifier C7 was obtained from Glen Research (from Glen Research CPG 20-2957). The C18 PEG was obtained from Glen Research (from Glen Research phosphoramidite 10-1918).

Assembly of Antibody-oligo Conjugates with Reporter Oligonucleotides.

The two antibody-oligo conjugates with their reporter were first assembled separately in a volume of 10 μl. Each assembly contained 0.5 μM (5 picomoles) of antibody-oligonucleotide conjugate and 0.15 μM of (15 pmoles) of complementary reporter oligonucleotide in 0.05 M Tris/HCl pH 8-0.01 M magnesium chloride. Each was incubated for at least 15 minutes at 4° C. before use in the detection reaction mixture.

Detection Reaction of Anti-PDGF-BB DPC Conjugates/Reporters with PDGF-BB

To conduct detection reaction, each reaction may contain in a volume of 50 μl: 10 μl of each conjugate assembly, prepared as described above, and variable amounts of PDGF-BB, in a buffer of 0.05 M Tris/HCl pH 8-0.01 M magnesium chloride-40% volume/volume formamide. The conjugates are present in this reaction mixture at 0.2 μM. Samples are incubated in the wells of a black 96-well microplate in a Wallac Victor Luminometer at 25° C. Fluorescence can be followed vs. time with excitation at 355 nm and emission at 460 nm.

Reactions typically may be carried out at 25° C., monitoring fluorescence generation at the wavelength optimums of the reaction product, 7-amino coumarin.

### Example 7

### Development and Clinical Significance of a BCR-ABL Fusion Protein Assay

A modular assay platform may be developed that provides broad applications for the specific in vitro and in vivo detection of proteins in complex biological milieus. This platform utilizes nucleic acid-templated chemistry (or DNA Programmed Chemistry, “DPC”) that enables the coupling of in situ protein recognition to de novo signal generation.

This approach is expected to have a significant impact for early diagnosis and therapeutic monitoring of cancer patients. For certain applications, this approach is advantageous by providing a simple homogeneous assay format to facilitate the development of point-of-care assays. For other applications, this approach may be used with flow cytometry, for example, or adapted for in vivo imaging.

A flow cytometry-based assay can be set up for BCR-ABL fusion protein to identify the subpopulation(s) of cells responsible for minimal residual disease (MRD) in CML patients. Heterogeneity within the same tumor has proven to be a major challenge to successful pharmacotherapy. Even in those cases, such as chronic myeloid leukemia CML (Goldman, et al., N Engl J Med 349 1451-1464 (2003); Sawyers, N Engl J Med 340 1330-1340 (1999)), where the cause has been elucidated at the molecular level (Rowley, Nature 243 290-293 (1973); Lugo, et al., Science 247 1079-1082 (1990)) and specific targeting (Druker, et al., Nat Med,. 2, 561-566 (1996); Deininger, et al., J. Blood, 105, 2640-2653 (2005)) has resulted in high rates of remission (Sawyers, et al., Blood, 99, 3530-3539 (2002); Kantarjian, et al., N Engl J Med 346, 645-652, (2002); Talpaz, et al., Blood 99, 1928-1937 (2002)), diverse mechanisms underlying primary and secondary resistance and disease persistence (Deininger, et al. Blood, 105, 2640-2653 (2005); Bhatia, et al. Blood 101, 4701-4707 (2003); Elrick, et al. Blood 105 1862-1866 (2005)) have, thus far, prevented high cure rates. While PCR-based approaches are quite sensitive for detecting MRD (Cortes, et al., Blood 102, 83-86 (2003)), they alone do not provide information about the molecular basis for the MRD in an individual patient. The protein-based assay described here may enable a specific cell-based approach using multiparameter flow cytometry (Irish, et al., Cell 118, 217-228 (2004)) to define MRD-causing cell profiles (e.g., status of influx and efflux pumps (Crossman, et al., Blood 106, 1133-1134 (2005); Thomas, et al., Blood 104 3739-3745 (2004); Mountford, et al., Blood 104 Abstract 716 (ASH) (2004)), integrin (Bueno-da-Silva, et al., Cell Death Differ. 10, 592-598 (2003)) and cytokine receptors (Chu, et al., Blood 103 3167-3174 (2004)), apoptosis modulators (Aichberger, et al., Blood 106 Abstract 1987 (ASH) (2005); Aichberger, et al., Blood 105, 33003-3311 (2005)), and signaling pathway activation (Jamieson, et al., N Engl J Med 351, 657-667 (2004)) in individual patients. Having this information enables the most informed clinical decisions and helps to define a focus for the development of new therapeutic strategies. By analogy, the results of this specific objective, focused on CML, can be extended to identify the subpopulations of cells responsible for MRD in ALL and AML patients. The inherent modularity of this protein assay approach should facilitate the development of flow cytometry-based assays for the E2A-PBX1, TEL/AML1, MLL/AF4 and PML/RARa, AML-ETO fusion proteins associated with ALL and AML, respectively.

Within the goal of extending scalar measurements to include the measurement of proteins in their functionally-relevant and/or (patho)physiological context, this approach is designed to allow the specific detection of homodimers, heterodimers, and protein-protein interactions indicative of the assembly of signal transduction complexes all in the presence of their monomeric counterparts. Thus this approach may be invaluable for the identification and validation of novel bona fide biomarkers that are mechanistically-linked to the pathophysiology of specific types of cancer. This may improve clinical trial design enabling the best treatment for the individual patient.

The fundamental principles of nucleic acid-templated chemistry and its inherent specificity can be used in complex biological environments for bio-detection under conditions where the structural and functional integrity of target analytes are preserved. The attachment of reactive groups to an analyte recognition element (e.g. antibodies, aptamers, or small molecules) directs chemical reaction to occur specifically at those sites containing the analyte of interest. Where the reactants are non-fluorescent and the reaction product is fluorescent, then a very low (“zero”) non-specific background signal can be obtained, allowing the measurement of analytes in complex environments without compromising specificity or sensitivity.

As represented in FIG. 4, a probe pair is used. Each member of the pair binds independently to the protein through its respective non-mutually exclusive recognition element. Each member of the pair contains a complementary deoxyoligonucleotide region designed to anneal to each other only at concentrations much higher than those used in the assay. However, when both probes are bound to the protein simultaneously, their effective concentrations are increased through proximity enabling DNA hybridization between the members of the pair. This protein-dependent hybridization event allows the attached non-fluorescent reactants to undergo a nucleic acid-templated reaction that generates a fluorescent product. In this way, analyte recognition involving two independent binding events triggers de novo signal generation. The protein-dependent hybridization between the members of the probe pair can serve as a point of avidity in the resulting ternary complex. The inherent specificity and affinity of each recognition element (e.g., antibody, aptamer, or low molecular weight ligand) alone is enhanced in this dual recognition assay format thereby improving their effective specificity and sensitivity.

One of the initial studies used the homodimeric BB form of PDGF as the analyte and employed aptamers as protein recognition elements conjugated to complementary deoxoligonucleotides. These, in turn, are attached to the non-fluorescent reactants triphenlyphosphine (5′-linked) and 7-azido-coumarin (3′-linked). Fluorescence generation, strictly dependent upon the presence of PDGF, was observed (FIG. 28). The excitation and emission spectra were indicative of 7-amino-coumarin, the expected product. Increasing concentrations of PDGF under conditions where the aptamer conjugates were not limiting, gave proportional increases in fluorescence signal. Maximal signal occurred when the ratio of complementary conjugates was 1:1. Furthermore, fluorescence generation was strictly dependent upon correct Watson-Crick base pairing of the complementary conjugates. Introduction of single base mismatched deoxoligonucleotides did not lead to PDGF-dependent fluorescence generation.

These data are consistent with the following model: the aptamer portion of the conjugates binds to PDGF inducing, through proximity, high effective molarities. This leads to the formation of a DNA duplex between the complementary pair of conjugates that, in turn, supports nucleic acid-templated reaction product formation. This enables the non-fluorescent precursors to react with each other to generate a signal that is directly coupled to analyte recognition. Fluorescence generation can be blocked using unconjugated aptamers that compete with the aptamer-deoxoligonucleotide-conjugates for PDGF binding. A 25-fold molar excess of unconjugated aptamer was required to compete with the conjugated aptamer to reduce signal generation by 50%.

Assay for Identifying BCR-ABL-Positive Cell Populations in CML Patients with Minimal Residual Disease: A protein assay applying the present invention that features dual recognition of an analyte triggering de novo signal generation can be used for the measurement of BCR-ABL in the context of a cell. Using multiparameter flow cytometry, this approach can identify the population of cells responsible for the MRD. This would be the critical step for defining the MRD-causing cell profile leading to a mechanism-based determination of the best course of treatment for individual patients.

Prepare anti-BCR and anti-ABL deoxyoligonucleotide-antibody DPC conjugates. A general protocol has been developed for conjugating either 5′- or 3′-aldehydic deoxyoligonucleotides to antibodies using the hetero-bifunctional reagent succinimidyl 6-hydrazinonicotinate acetone hydrazone (SANH) based upon published protocols, e.g., (www.solulink.com). The conjugates have been purified using gel exclusion chromatography followed by anion exchange chromatography and the degree of oligonucleotide conjugation per antibody molecule has been quantitated using SYBR Gold fluorescence enhancement. This approach can be applied to commercially available polyclonal and monoclonal anti-BCR and anti-ABL antibodies.

A high quality monoclonal antibody facility can also help generate new antibodies to BCR and ABL. Molecular modeling capabilities may be applied to predict epitopes that are: 1) present in the two clinically relevant fusion protein subtypes, B3/A2 and B2/A2, 2) topologically oriented to enable antibody pairs to bind favorably, 3) likely to be insensitive to fusion protein dimerization, Gleevec binding, known resistant-conferring mutations, and perhaps substrate binding.

Detection of purified BCR-ABL fusion protein. The probe pairs generated can be used to develop an assay for BCR-ABL fusion protein in an analogous manner to the PDGF assay described above. One member of the probe pair will have anti-BCR antibody as its recognition element while the complementary member will utilize anti-ABL as its recognition element. BCR-ABL (B3/A2) fusion protein has been expressed from a p210(bcr-abl)baculovirus expression construct generated by splicing together bcr and abl cDNAs with a bcr-abl junction fragment from K562 cDNA and placing it in pDEST8. Full length BCR and ABL can be used to ensure that the assay is specific for the fusion protein. The limit of detection is determined using the purified B3/A2 fusion protein and fusion protein derived from B2/A2 and B3/A2-positive cell lysates. The extent of interference from BCR-ABL-negative cell lysates can also be determined.

Reactions for fluorophor generation. Reporter chemistry described here in may be applied for the generation of fluorophor. Preferably the chemistry will yield fluorophors with excitation maxima >500nm, emission maxima >600nm with quantum yields greater than 0.5 from relatively stable DPC-based precursors having no appreciable fluorescence themselves.

Flow Cytometry Assay for Identifying BCR-ABL-Positive Cell Populations from CML Patients.

Prepare anti-BCR and anti-ABL deoxyoligonucleotide conjugates that have standard fluorescent dyes used for flow cytometry linked in place of the nucleic acid-templated reactive compound (reactants). These can be used as positive controls for optimizing the fixation and permeabilization conditions to ensure and quanitate intracellular access of the detection probe pairs. Human myeloid patient-derived cell lines can be used. Initial conditions may be based upon protocols implemented for studying activation of intracellular signal transduction pathways (Jamieson, et al., N Engl J Med 351, 657-667 (2004)) using activation-state specific kinase antibodies (Irish, et al., Cell 118, 217-228 (2004)). Based upon the results, a probe pair optimized for flow cytometry are designed and prepared.

A prototype DPC-based flow cytometry assay can be developed. Initially, a variety of B3/A2 and B2/A2 positive patient-derived cell lines that include K562 cells can be used. The specificity and sensitivity can be determined by diluting these positive cells with BCR-ABL negative cells. The objective is to detect 10-30 BCR-ABL-positive cells in the presence of 1 million BCR-ABL-negative cells. Once this objective is achieved, the assay can be further validated with samples from CML patients and healthy volunteers. The specificity and sensitivity of this assay can be compared to validated methods that utilize fluorescence in situ hybridization (FISH) (Schoch, et al., Leukemia 16 53-59 (2002)) and DNA/RNA polymerase chain reaction (PCR) (Elrick, et al., Blood 105 1862-1866 (2005)). Therefore, a fluorescence activated cell sorting (FACS) analysis on samples from several patients can be done.

There is considerable evidence emerging that suggests some of the mechanisms responsible for primary and secondary resistance to Gleevec and disease persistence in patients with CML. In addition to mutations in the kinase domain of BCR-ABL, influx and efflux pumps, integrin and cytokine receptors, apoptosis modulators, and signaling pathways involving MAPkinase and beta-catenin have been implicated. Guided by these results, it should be possible to establish MRD-causing cell profiles in individual patients by using the proposed BCR-ABL protein assay in a multi-parameter flow cytometry format. This approach would be analogous to cell profiling of potentiated phospho-protein networks in cancer cells. The “biosignatures” of these MRD-causing cells could then be compared among individual patients before and in response to various therapeutic regimens. In light of the diversity of potential mechanisms preventing cures, cell profiling could prove invaluable in ensuring that each individual patient receives the most appropriate pharmacotherapy. Irish, et al., Cell 118, 217-228 (2004); Crossman, et al., Blood 106, 1133-1134 (2005); Thomas, et al., Blood 104 3739-3745 (2004); Mountford, et al., Blood 104 Abstract 716 (ASH) (2004); Bueno-da-Silva, et al., Cell Death Differ. 10, 592-598 (2003); Chu, et al., Blood 103 3167-3174 (2004); Aichberger, et al., Blood 106 Abstract 1987 (ASH) (2005); Aichberger, et al., Blood 105, 33003-3311 (2005); Jamieson, et al., N Engl J Med 351, 657-667 (2004).

Various and general aspects of nucleic acid-templated chemistry are discussed in detail below. Additional information may be found in U.S. Patent Application Publication Nos. 2004/0180412 A1 (U.S. Ser. No. 10/643,752) by Liu et al. and 2003/0113738 A1 (U.S. Ser. No. 10/101,030) by Liu et al.

### Example 8

### Nucleic Acid-Templated Generation of Various Dyes

Three oligonucleotides were prepared using standard phosphoramidite chemistry and purified by reversed-phase C18 column (Glen Research, Sterling Va., USA). Oligonucleotides bearing 5′-amino groups (EDC2 and EDC3) were prepared using 5′-Amino-Modifier 5 and Oligonucleotides bearing 3′-amino groups (EDC1) were prepared using 3′-Amino-Modifier C7 CPG (Glen Research, Sterling Va., USA). Concentration of the DNA and heterocyclic conjugated DNA was determined by UV absorbance at 260 nm. The contribution of the UV absorbance at 260 nm from the heterocyclic moiety in the heterocyclic conjugated DNA was negligeable and was not considered.

| | | |
|---|---|---|
| Oligo# | sequence (5′-3′) | SEQ. ID. |

| | | |
|---|---|---|
| EDC1 | GTGGTAGTTGGAGCT-NH2 | (SEQ. ID. NO. 61) |
| EDC2 | H2N-AGCTCCAACTACCAC | (SEQ. ID. NO. 62) |
| EDC3 | H2N-AGATCCCACTAGCAC | (SEQ. ID. NO. 63) |

Synthesis of DNA conjugated heterocyclic precursors for aldol condensation. Scheme 14 provides two examples of the synthesis of DNA conjugated heterocyclic precursors for aldol condensation.

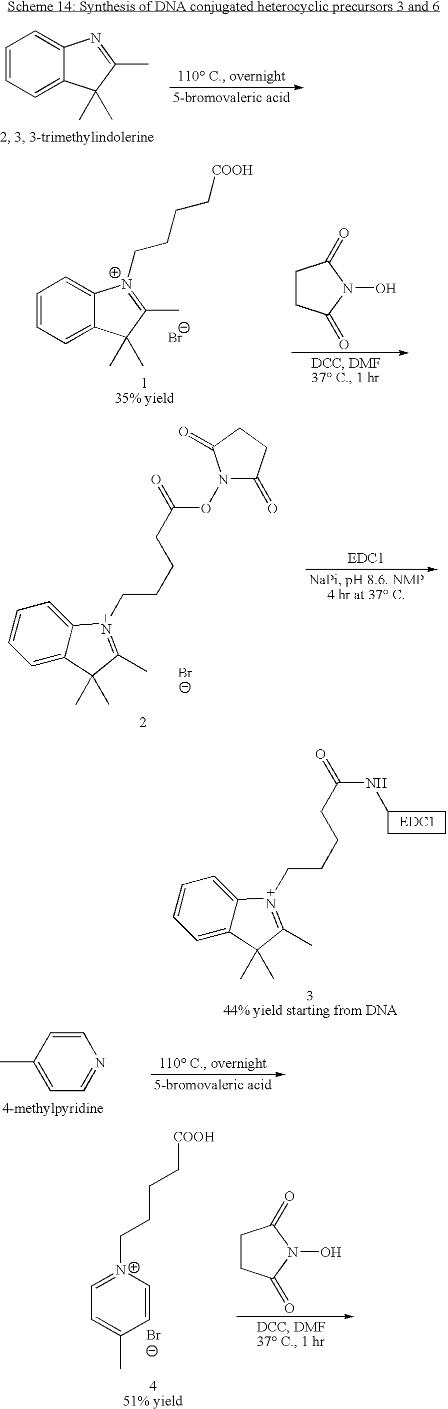

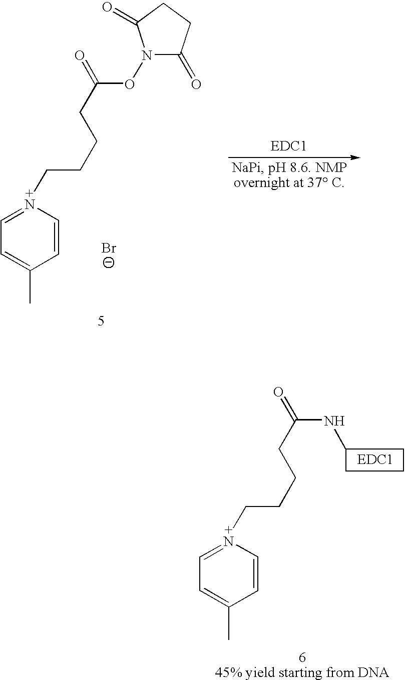

Synthesis of compound 1: To 5-bromovaleric acid (2.435 g, 13.45 mmole) was added 2,3,3-trimethylindolenine (2.141 g, 13.45 mmole). The reaction mixture was heated with rigorous stirring at 110° C. overnight. The dark red sticky oil obtained was transferred to a Gregar extractor and extracted with EtOAc overnight. A light red solid was obtained. The solid was redissolved in 30 mL of MeOH. MeOH was removed under reduced pressure and the remaining residue was treated with 10 mL of EtOAc. Browish solid was precipitated out and filtrated. The solid was washed with 2×50 mL of acetone and 2×100 mL of EtOAc. Total 1.590 g of light brownish solid was obtained (35% yield). ¹H NMR (DMSO) δₚₚₘ: 7.98 (m, 1H), 7.84 (m, 1H), 7.61 (m, 2H), 4.49 (t, 2H), 2.84 (s, 3H), 2.30 (t, 2H), 1.84 (m, 2H), 1.63 (m, 2H), 1.53 (s, 6H). MALDI-MS (positive mode): 260.2419.

Synthesis of compound 2: Compound 1 (0.1 g, 0.294 mmole), N-hydroxy succimide (0.068 g, 0.588 mmole) and N,N′-dicyclohexylcarbodiimide (DCC) (0.085 g, 0.411 mmole) were dissolved in 1.5 mL of DMF. The reaction mixture was stirred at 37° C. for 1 hr. The precipitated dicyclohexylurea (DCU) was removed by filtration, and the filtrate was treated with 15 mL of ether. Light orange solid was washed three times with 10 mL of ether and dried under vacuum for several hours. The solid obtained was used directly for the next reaction. MALDI-MS (positive mode): 357.1590.

Synthesis of compound 3: To a 1.5 mL of centrifugation vial containing 20 nmole of DNA (EDC1) was added 41.6 μL of 0.1 M sodium phosphate buffer (NaPi), pH 8.6, 41.6 μL of compound 2 in NMP (96 mM) and 41.6 μL of NMP. The vial was placed in a shaker and shaked for 4 hr at 37° C. The reaction mixture was desalted by gel filtration using Sephadex G-25 and then purified by reversed-phase C18 column. Total 8.81 nmole of desired product was obtained (44% yield). LC-MS (negative mode): Calcd for C₁₇₂H₂₂₁N₆₀O₉₆P₁₅ (monoisotopic): 1024.4070 [M−5H]⁻⁵; 1280.7473 [M−4H]⁻⁴ Found: 1024.3986 [M−5H]⁵⁻; 1280.7473 [M−4H]⁴⁻

Synthesis of compound 4 (similar procedure of synthesizing compound 1): 4-methylpyridine (1.245 g, 13.37 mmole) and 5-bromovaleric acid (2.4203 g, 13.37 mmole) was heated with rigorous stirring at 110° C. overnight. 50 mL of EtOAc was added to the sticky oil. The burgundy solid obtained was broken up and washed extensively with EtOAc and Acetone. The solid was filtrated and dried under vacuum to afford 1.886 g of 4 as white solid (51% yield). ¹H NMR (CD₃OD) δₚₚₘ: 8.84 (d, 1H), 7.96 (d, 1H), 4.6 (t, 2H), 2.69 (s, 3H), 2.40 (t, 2H), 2.05 (t, 2H), 1.65 (m, 2H). MALDI-MS (positive mode): 194.1457.

Synthesis of compound 5: Compound 5 was synthesized following the same procedure of synthesis compound 2 and was used directly for DNA conjugation without ether precipitation. MALDI-MS (positive mode): 291.1605.

Synthesis of compound 6: Following the general procedure of DNA labeling, 20 nmole of DNA (EDC1) was reacted with compound 5 overnight at 37° C. to afford 9.05 nmole of pure pyridinium conjugated DNA 6 (45% yield). LC-MS (negative mode): Calcd for C₁₆₈H₂₁₇N₆₀O₉₆P₁₅ (monoisotopic): 1264.2385 [M−4H]⁴⁻; 1685.9872 [M−3H]³⁻ Found: 1264.2313 [M−4H]⁴⁻; 1685.9871 [M−3H]³⁻

Synthesis of DNA-conjugated aldehyde precursors for aldol condensation and Wittig reaction. Scheme 15 and Scheme 16 shows two examples of introducing the acid functionality to heterocyclics through N-quaternization. Scheme 17 gives one example of converting a cyano group to an acid group for DNA conjugation.

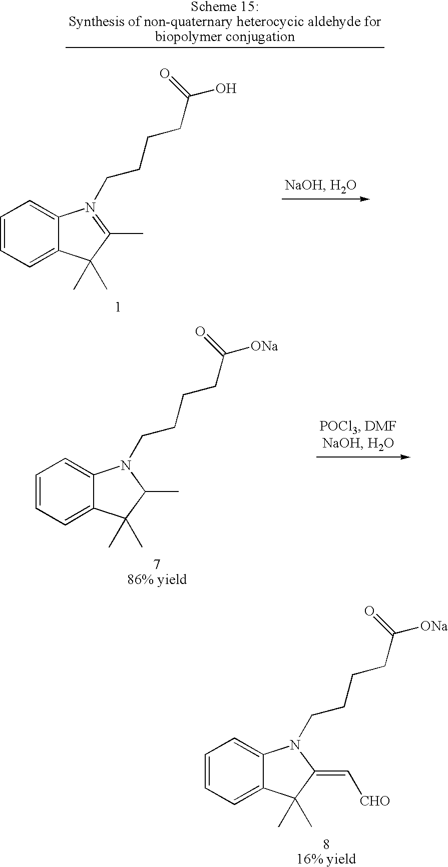

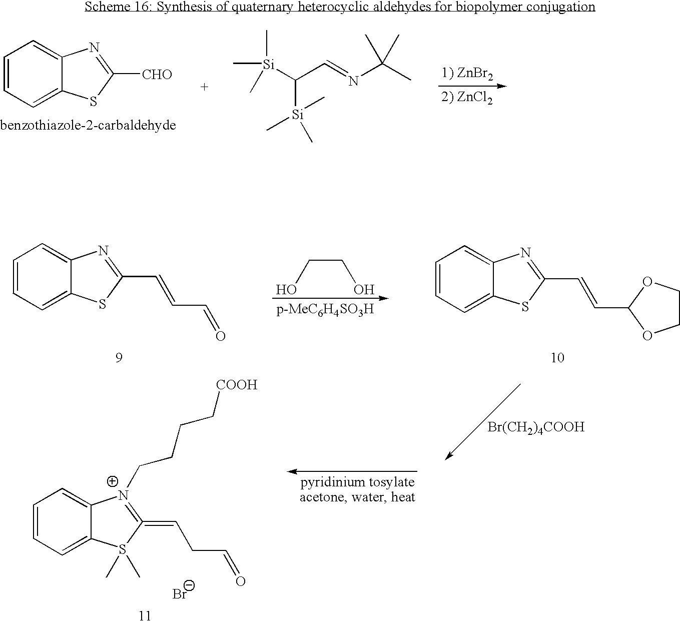

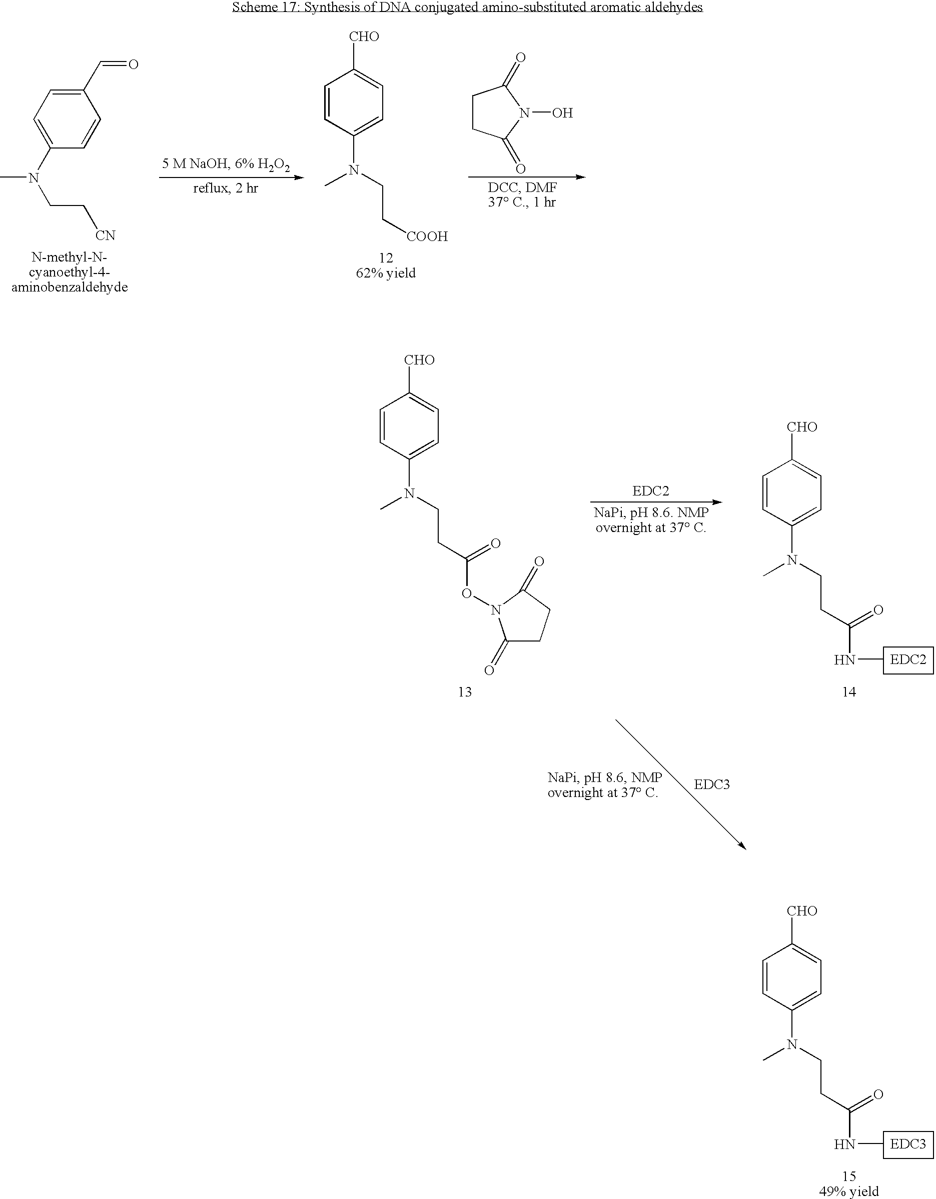

Synthesis of compound 7: A mixture of 1 (0.25 g, 0.735 mmol) and sodium hydroxide (0.039 g, 0.970 mmol) were dissolved in 1.9 mL of water and stirred vigorously at RT. After 3 hour, the reaction mixture was loaded directly onto a 4.3 g of RediSep reversed-phase C18 column. The column was first washed with water to get rid of excess salt and then acetonitrile to elute the product. Total 0.178 g of product was obtained (86% yield). ¹H NMR (DMSO) δₚₚₘ: 7.11 (dd, 1H), 7.05 (dt, 1H), 6.66 (dt, 1H), 6.61 (dd, 1H), 3.85 (d, 2H), 3.45 (t, 2H), 1.48 (m, 4H), 1.88 (t, 2H), 1.24 (s, 6H). (Wang, et al., Dyes and Pigments 2003, 57, 171-179).

Synthesis of compound 8: In a 4 mL of glass vial with PTFE/silicone septa under Ar was added 300 μL of anhydrous DMF. Vial and its contents are cooled in an ice-salt bath for 10 minutes, then 84 μL of phosphorous oxychloride was added. After another 10 minutes, a solution of compound 7 (0.15 g, 0.533 mmole) in 300 μL of DMF was added slowly. The solution became viscous. The vial was transferred to a shaker preheated at 35° C. and shaked for another 45 minute. 200 mg of ice was added to the reaction mixture with careful stirring followed by 450 mg of NaOH in 1.2 mL of water. The resulting suspension was heated rapidly to the boiling point and allowed to cool to RT. The resulting mixture was first purified by a 12 g of RediSep reversed-phase C18 column on a CombiFlash Companion Chromatography system (Teledyne ISCO) (acetonitrile/water) and then by semi-preparative thin layer chromatography (solvent system: 70:29:1 CH₂Cl₂:MeOH:AcOH). Total 26 mg of pure product was obtained (16% yield). ¹H NMR (CD₃OD) δₚₚₘ: 9.79 (d, 1H), 7.35 (d, 1H), 7.31 (t, 1H), 7.11 (t, 2H), 5.51 (d, 1H), 3.85 (t. 2H), 2.25 (t, 2H), 1.73 (m, 4H), 1.65 (s, 6H). (Wang, et al., Dyes and Pigments 2003, 57, 171-179)

Synthesis of compound 9: To a solution of benzothiazole-2-carbaldehyde (102 mg, 0.623 mmole) and ZnBr₂ (140 mg, 0.623 mmol) in 1.5 mL of THF was added a solution of (E)-N-(2,2-bis(trimethylsilyl)ethylidene-2-methylpropan-2-amine (167 mg, 0.685 mmole) in THF (0.3 mL) dropwise at RT. After being stirred for 2 hr, the resulting mixture was hydrolyzed by addition of an aqueous solution of ZnCl₂ (297 mg in 2.2 mL of water) and ether (2.56 mL) (the extent of the hydrolysis was monitored by HPLC analysis). THF was removed by a stream of Ar. The aqueous layer was extracted with CH₂Cl₂. After drying over MgSO₄, the crude product was purified by a 12 g RediSep silica-gel column on a CombiFlash Companion chromatography system (EtOAc/hexanes). 97 mg of product was obtained (82% yield). ¹H NMR (CD₃Cl) δₚₚₘ: 9.8 (d, 1H), 8.1 (d, 1H), 7.9 (d, 1H), 7.7 (d, 1H), 7.6 (t, 1H), 7.5 (t, 1H), 6.9 (dd, 1H). (Bellassoued, et al., A. J. Org. Chem. 1993, 58, 2517-2522)

Synthesis of compound 12: In a 50 mL of round-shaped flask containing N-methyl-N-cyanoethyl-4-aminobenzaldehyde (1.024 g, 5.44 mmole) was added 27.2 mL of 5 N NaOH solution and 6.8 mL of 30% H₂O₂. The reaction mixture was refluxed for 2 hr. After cooling down, the reaction mixture was neutralized by the addition of concentrated HCl (37% w.t.) and extracted with 2×100 mL of EtOAc and 1×100 mL of CH₂Cl₂. The organic layers were combined and washed once with 50 mL of brine and concentrated to dryness. The crude product was purified by a 40 g RediSep silica-gel column on a CombiFlash Companion chromatography system (EtOAc/MeOH). Total 0.702 g of light pinkish solid was obtained (62%). Electrospray MS: M+H 208.0735. (Brady, et al., J. Biol. Chem. 2001, 276, 18812-18818)

Synthesis of compound 13: Compound 13 was synthesized following the same procedure of synthesizing compound 2 and was used directly for DNA conjugation without ether precipitation.

Synthesis of compound 14: Following the general procedure of DNA labeling, 20 nmole of DNA (EDC2) was reacted with compound 13 overnight at 37° C. to afford 8.8 nmole of 14 (44%). LC-MS: Calcd for C₁₅₈H₂₀₄N₅₇O₉₁P₁₅ (monoisotopic): 1203.9710 [M−4H]⁴⁻; 1605.6306 [M−3H]³⁻ Found: 1203.9664 [M−4H]⁴⁻; 1605.6305 [M−3H]³⁻

Synthesis of compound 15: Following the general procedure of DNA labeling, 20 nmole of DNA (EDC3) was reacted with compound 13 overnight at 37° C. to afford 9.7 nmole of 15 (49%). LC-MS: Calcd for C₁₅₉H₂₀₄N₅₉O₉₁P₁₅ (monoisotopic): 1213.9725 [M−4H]⁴⁻; 1618.9660 [M−3H]³⁻ Found: 1213.9620 [M−4H]⁴⁻; 1618.9590 [M−3H]³⁻

Synthesis of precursors for Wittig or Horner reaction. An example of synthsizing amino substituted aromatic phosphonium salt was presented (Scheme 18) here using a convenient one-pot procedure without isolation of halide reagent.

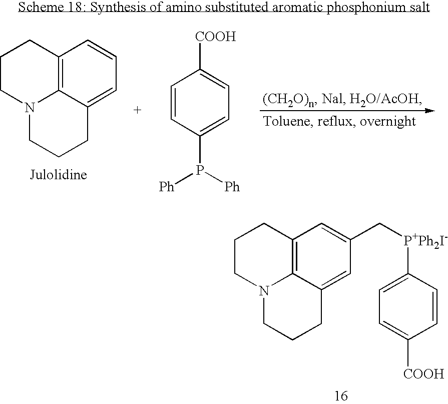

Synthesis of compound 16: To a solution of julolidine (0.97 g, 5.60 mmol), 4-(diphenylphosphino)benzoic acid (1.715 g, 5.60 mmol) and paraformaldehyde (0.168 g) in 8 mL of toluene was added NaI (0.84 g, 5.60 mmol), water (0.397 mL) and HOAc (1.13 mL). The mixture was refluxed for overnight. After addition of 15 mL of water, the reaction mixture was extracted twice with CH₂Cl₂. The combined CH₂Cl₂ layer was washed twice with saturated NaHCO₃, then once with water and dried over Na₂SO₄. After removing the solvent, the residue was purified by a 40 g RediSep silica-gel column on a CombiFlash Companion chromatography system (EtOAc/hexanes). 1.77 g of yellow solid obtained (51% yield). ¹H NMR (CD₃Cl) δₚₚₘ: 8.01 (dd, 2H), 7.86 (t, 2H), 7.77 (m, 4H), 7.62 (m, 4H), 7.52 (m, 2H), 6.20 (s, 2H), 4.77 (d, 2H), 3.03 (t, 4H), 2.36 (t, 4H), 1.75 (m, 4H). MS (positive mode): 492.205

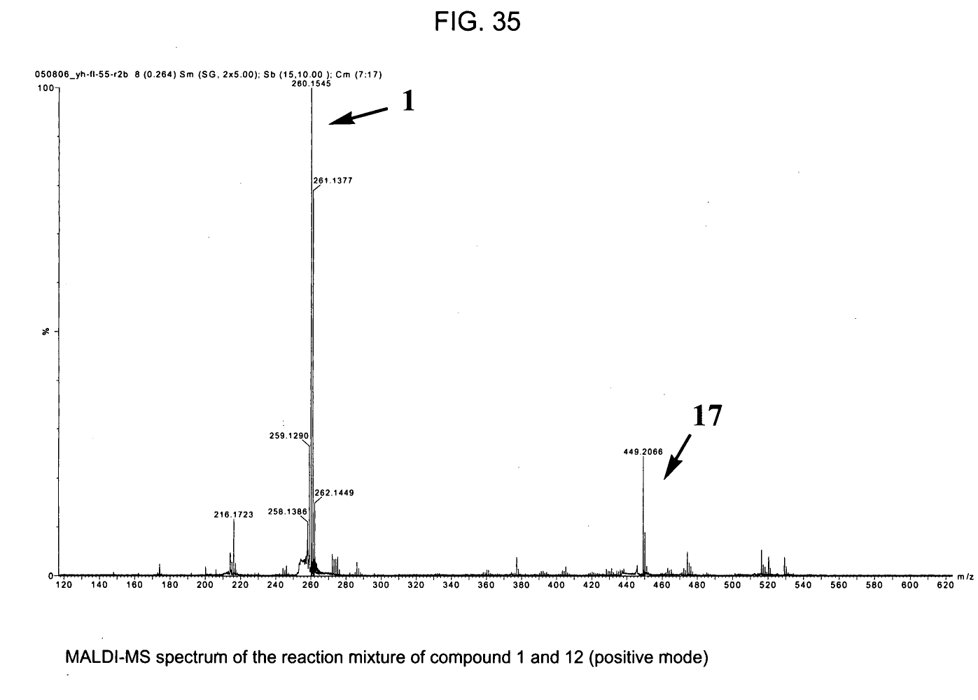

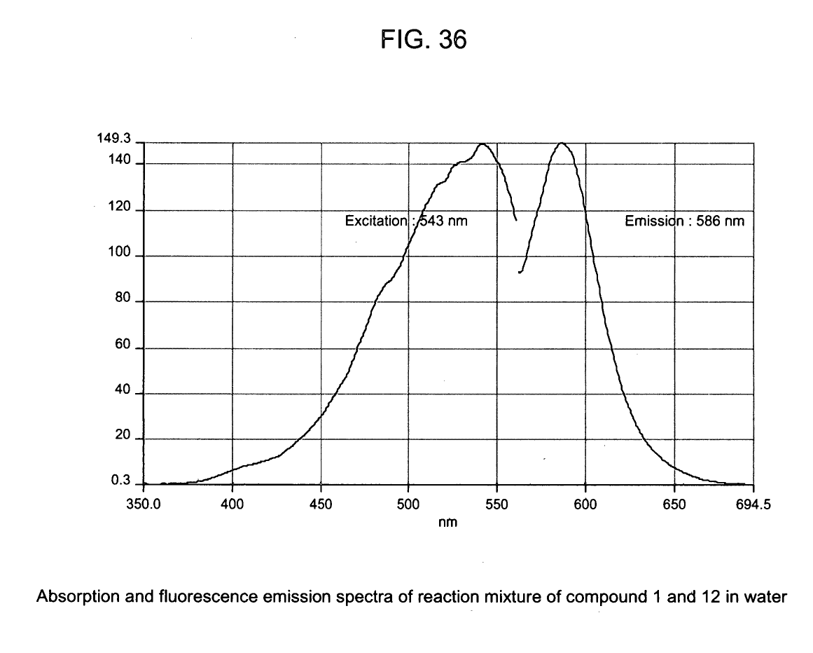

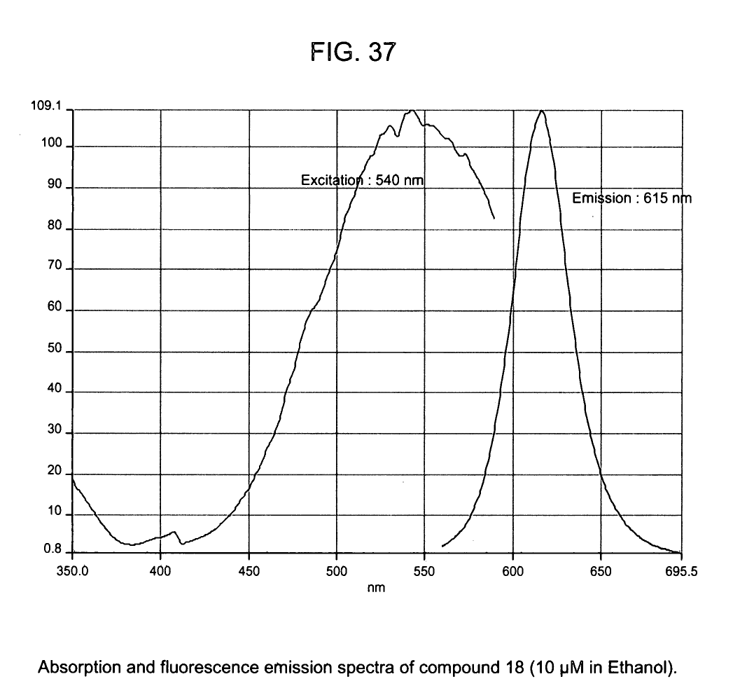

Polymethine generation through aldol condensation in aqueous condition. Although most of the previous literature data indicate the aldol condensation only happens under harsh condition (reflux ethanol under basic condition), we show here two examples where N-quaternary heterocyclic precursor bearing active-hydrogen participates into aldol condensation under mild aqueous condition. In Scheme 19, after mixing compound 1 and 12 in aqueous buffer for just few minutes, a deep purple color was observed. Mass analysis indicates the Aldol condensation product is formed (FIG. 35) and the diluted reaction mixture shows the characteristic hemicyanine dye fluorescence (FIG. 36, Excitation: 543 nm and Emission: 586 nm). Scheme 20 illustrates another example of aldol condensation under aqueous conditions where the purified hemicyaine product exhibit fluorescence at 615 nm (Excitation at 540 nm, FIG. 37).

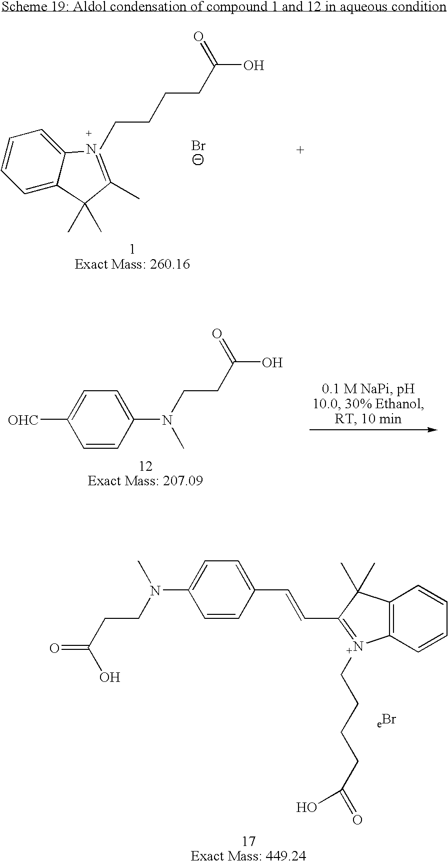

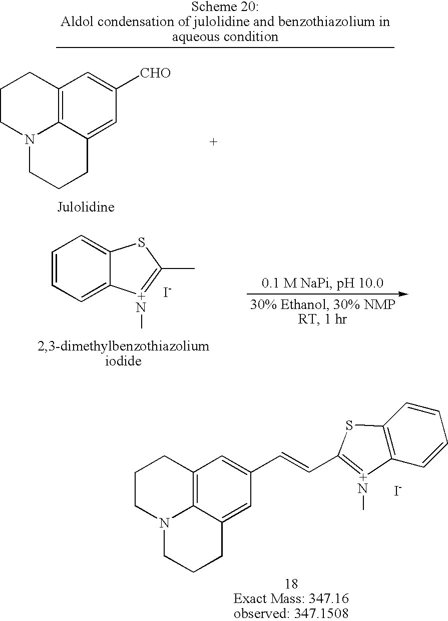

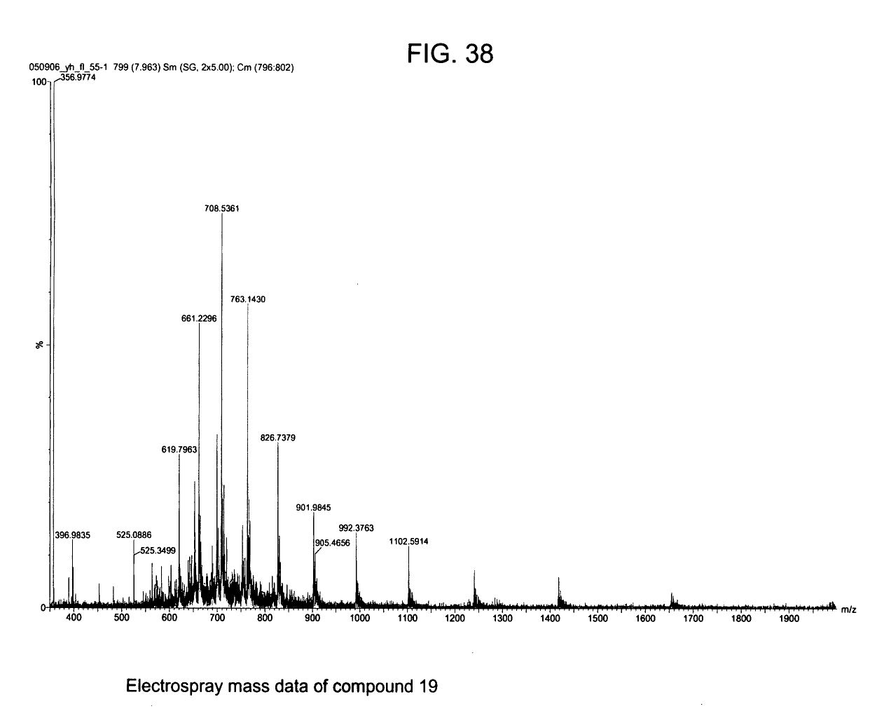

Polymethine generation through nucleic acid-templated reaction. Scheme 21 illustrates an example of the nucleic acid-templated aldol condensation between compound 3 and compound 14. After overnight incubation at 37° C., LC-MS analysis of the product shows the polymethine dye formation (FIG. 38).

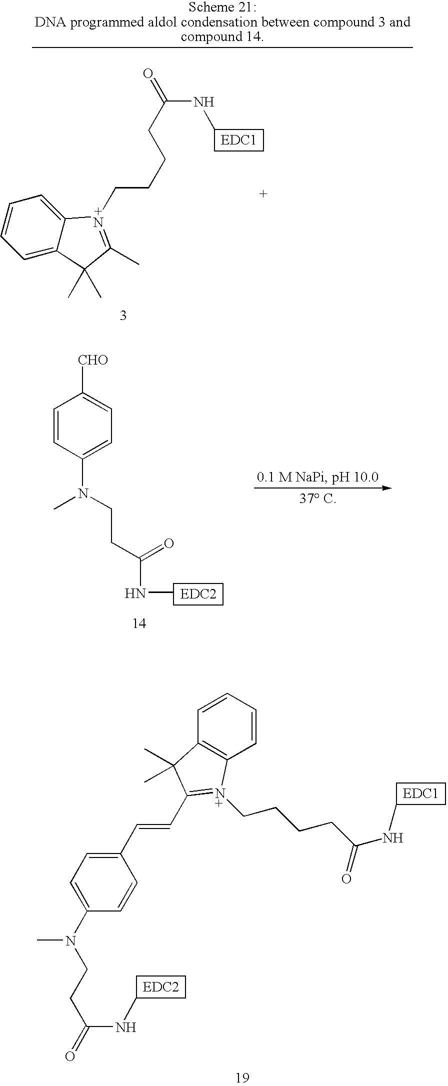

### INCORPORATION BY REFERENCE

The entire disclosure of each of the publications and patent documents referred to herein is incorporated by reference in its entirety for all purposes to the same extent as if each individual publication or patent document were so individually denoted.

### Equivalents

The invention may be embodied in other specific forms without departing form the spirit or essential characteristics thereof. The foregoing embodiments are therefore to be considered in all respects illustrative rather than limiting on the invention described herein, Scope of the invention is thus indicated by the appended claims rather than by the foregoing description, and all changes that come within the meaning and range of equivalency of the claims are intended to be embraced therein.

What is claimed is:

1. A method for detecting a target nucleotide sequence, the method comprising:
(a) providing (1) a first probe comprising (i) a first nucleotide sequence and (ii) a first reactive group linked to the first oligonucleotide sequence, and (2) a second probe comprising (i) a second oligonucleotide sequence and (ii) a second reactive group linked to the second oligonucleotide sequence, wherein the first oligonucleotide sequence and the second oligonucleotide sequence are complementary to two separate regions of the target nucleotide;
(b) combining the first probe and the second probe with a sample to be tested for the presence of the target nucleotide sequence under conditions where the first probe and the second probe hybridize to their respective complementary regions of the target nucleotide sequence if present in the sample thereby bringing into reactive proximity the first reactive group and the second reactive group; and
(c) detecting a reaction between the first reactive group and the second reactive group thereby determining the presence of the target nucleotide sequence.

2. The method of claim 1 wherein the reaction product of the first reactive group and the second reactive group comprises a fluorescent or a chromophoric moiety.

3. The method of claim 2 wherein the reaction product of the first reactive group and the second reactive group comprises a fluorescent moiety.

4. The method of claim 3 wherein the fluorescent moiety is selected from the group consisting of cyanine dyes, hemicyanine dyes and coumarin dyes.

5. The method of claim 3 wherein the fluorescent moiety is a polymethine dye.

6. The method of claim 1 wherein the reaction of the first reactive group and the second reactive group is by chemically coupling the first reactive group and the second reactive group.

7. The method of claim 2 wherein the fluorescent or chromophoric moiety is covalently linked to one or both of the first probe and the second probe.

8. The method of claim 2 wherein the fluorescent or chromophoric moiety is covalently linked to neither the first probe nor the second probe.

9. The method of claim 1 wherein the reaction of the first reactive group and the second reactive group results in the release of an enzyme co-factor.

10-21. (canceled)

22. A method for detecting a biological target, the method comprising:
(a) providing a first probe, the first probe comprising (1) a first binding moiety having binding affinity to the biological target, (2) a first oligonucleotide sequence, and (3) a first reactive group associated with the first oligonucleotide sequence;
(b) providing a second probe, the second probe comprises (1) a second binding moiety having binding affinity to the biological target, (2) a second oligonucleotide sequence, and (3) a second reactive group associated with the second oligonucleotide sequence, wherein the second oligonucleotide is capable of hybridizing to the first oligonucleotide sequence and the second reactive group is reactive to the first reactive group when brought into reactive proximity of one another;
(c) combining the first probe and the second probe with a sample to be tested for the presence of the biological target under conditions where the first and the second binding moieties bind to the biological target;
(d) allowing the second oligonucleotide to hybridize to the first oligonucleotide to bring into reactive proximity the first and the second reactive groups; and
(e) detecting a reaction between the first and the second reactive groups thereby determining the presence of the biological target.

23. The method of claim 22 wherein the first probe further comprises a first linker between the first binding moiety and the first oligonucleotide sequence.

24. The method of claim 22 wherein the second probe further comprises a second linker between the second binding moiety and the second oligonucleotide sequence.

25. The method of claim 22 wherein the biological target is a protein.

26. The method of claim 22 wherein the biological target is an autoantibody.

27. The method of claim 22 wherein the biological target is a cell.

28. The method of claim 22 wherein at least one of the first and the second binding moieties is an antibody to the biological target.

29. The method of claim 22 wherein both the first and the second binding moieties are antibodies to the biological target.

30. The method of claim 22 wherein at least one of the first and the second binding moieties is not an antibody to the biological target.

31. The method of claim 22 wherein at least one of the first and the second binding moieties is an aptamer that binds to the biological target.

32. The method of claim 22 wherein both the first and the second binding moieties are aptamers that binds to the biological target.

33. The method of claim 22 wherein at least one of the first and the second binding moieties is a small molecule binder.

34. The method of claim 22 wherein both the first and the second binding moieties are small molecule binders.

35. The method of claim 22 wherein the first oligonucleotide sequence and the second oligonucleotide sequence comprise a 6 to 30-base complimentary region.

36. The method of claim 22 wherein the reaction between the first and the second reactive groups produces a fluorescent moiety.

37. The method of claim 22 wherein the reaction between the first and the second reactive groups produces a chemiluminescent or a chromophoric moiety.

38. The method of claim 22 wherein in the absence of the biological target in the sample, substantially no detectable reaction occurs between'the first and the second reactive groups.

39. A method for detecting a biological target, the method comprising:
(a) providing a binding complex of the biological target with a first probe, the first probe comprising (1) a first binding moiety having binding affinity to the biological target, (2) a first oligonucleotide sequence, and (3) a first reactive group associated with the first oligonucleotide sequence;
(b) contacting the binding complex of (a) with a second probe, the second probe comprising (1) a second binding moiety having binding affinity to the biological target, (2) a second oligonucleotide sequence, and (3) a second reactive group associated with the second oligonucleotide sequence, wherein the second oligonucleotide is capable of hybridizing to the first oligonucleotide sequence and the second reactive group is reactive to the first reactive group when brought into reactive proximity of one another;
(c) allowing the second oligonucleotide to hybridize to the first oligonucleotide to bring into reactive proximity the first and the second reactive groups; and
(d) detecting a reaction between the first and the second reactive groups thereby determining the presence of the biological target.

40. A method for detecting the presence of a biological target, the method comprising:
(a) binding to the biological target a first probe and a second probe, wherein
(1) the first probe comprises (i) a first binding moiety having binding affinity to the biological target, (ii) a first oligonucleotide sequence, and (iii) a first reactive group associated with the first oligonucleotide sequence and
(2) the second probe comprises (i) a second binding moiety having binding affinity to the biological target, (ii) a second oligonucleotide sequence, and (iii) a second reactive group associated with the second oligonucleotide sequence, wherein the second oligonucleotide is capable of hybridizing to the first oligonucleotide sequence and the second reactive group is reactive to the first reactive group when brought into reactive proximity of one another;
(b) allowing the second oligonucleotide to hybridize to the first oligonucleotide sequence thereby bringing into reactive proximity the first and the second reactive groups; and
(c) detecting a reaction between the first and the second reactive groups thereby determining the presence of the biological target.

41. The method of claim 40 wherein the first probe further comprises a first linker between the first binding moiety and the first oligonucleotide sequence.

42. The method of claim 40 wherein the second probe further comprises a second linker between the second binding moiety and the second oligonucleotide sequence.

43. The method of claim 40 wherein the biological target is a protein.

44. The method of claim 40 wherein the biological target is an autoantibody.

45. The method of claim 40 wherein the biological target is a cell.

46. The method of claim 40 wherein at least one of the first and the second binding moieties is an antibody to the biological target.

47. The method of claim 40 wherein both the first and the second binding moieties are antibodies to the biological target.

48. The method of claim 40 wherein at least one of the first and the second binding moieties is not an antibody to the biological target.

49. The method of claim 40 wherein at least one of the first and the second binding moieties is an aptamer that binds to the biological target.

50. The method of claim 40 wherein both the first and the second binding moieties are aptamers that bind to the biological target.

51. The method of claim 40 wherein at least one of the first and the second binding moieties is a small molecule binder.

52. The method of claim 40 wherein both the first and the second binding moieties are small molecule binders.

53. The method of claim 40 wherein the first oligonucleotide sequence and the second oligonucleotide sequence comprise a 6 to 30-base complimentary region.

54. A method for detecting a biological target, the method comprising:
(a) providing a first probe, the first probe comprises (1) a first binding moiety having binding affinity to the biological target, and (2) a first oligonucleotide zip code sequence;
(b) providing a second probe, the second probe comprises (1) a second binding moiety having binding affinity to the biological target, and (2) a second oligonucleotide zip code sequence,
wherein the first probe is hybridized to a first reporter probe comprising (1) an anti-zip code sequence of oligonucleotides complementary to the first oligonucleotide zip code sequence, (2) a first reporter oligonucleotide, and (3) a first reactive group;
wherein the second probe is hybridized to a second reporter probe comprising (1) an anti-zip code sequence of oligonucleotides complementary to the second oligonucleotide zip code sequence, (2) a second reporter oligonucleotide, and (3) a second reactive group; wherein the second reporter oligonucleotide is capable of hybridizing to the first reporter oligonucleotide sequence and the second reactive group is reactive to the first reactive group when brought into reactive proximity of one another;
(c) contacting the first and the second probes with a sample to be tested for the presence of the biological target;
(d) allowing the first and the second probes to bind to the biological target if present in the sample, whereby the second reporter oligonucleotide hybridizes to the first reporter oligonucleotide sequence to bring into reactive proximity the first and the second reactive groups; and
(e) detecting a reaction between the first and the second reactive groups thereby determining the presence of the biological target.

55. The method of claim 54 wherein the first and the second binding moieties are antibodies.

56. The method of claim 54 wherein the first and the second binding moieties are aptamers.

57. The method of claim 54 wherein the first and the second binding moieties are small molecule binders.

58. The method of claim 54 wherein the reporter chemistry between the first and second reactive groups generate a polymethine or a derivative thereof.

59. The method of claim 54 wherein the reporter chemistry between the first and second reactive groups generate a cyanine or a derivative thereof.

60. The method of claims 54 wherein the reaction between the first and the second reactive groups is a Wittig reaction.

61. The method of claims 54 wherein the reaction between the first and the second reactive groups is an aldol condensation reaction.

62-71. (canceled)


SEQUENCE LISTING (65 sequences)

SEQ ID NO: 1 - DNA, 10 bp, Artificial Sequence
  Probe oligonucleotide
tgtaggtaac

SEQ ID NO: 2 - DNA, 10 bp, Artificial Sequence
  Probe oligonucleotide
gttacctaca

SEQ ID NO: 3 - DNA, 18 bp, Artificial Sequence
  Probe oligonucleotide
cttcttcatgtaggtaac

SEQ ID NO: 4 - DNA, 18 bp, Artificial Sequence
  Probe oligonucleotide
cttcttcagttacctaca

SEQ ID NO: 5 - DNA, 18 bp, Artificial Sequence
  Oligonucleotide
caatggatgtacttcttc

SEQ ID NO: 6 - DNA, 18 bp, Artificial Sequence
  Oligonucleotide
acatccattgacttcttc

SEQ ID NO: 7 - DNA, 18 bp, Artificial Sequence
  Oligonucleotide
caatggatgtacttcttc

SEQ ID NO: 8 - DNA, 18 bp, Artificial Sequence
  Oligonucleotide
acatcctttgacttcttc

SEQ ID NO: 9 - DNA, 10 bp, Artificial Sequence
  Oligonucleotide reporter sequence
cgaatttata

SEQ ID NO: 10 - DNA, 10 bp, Artificial Sequence
  Oligonucleotide reporter sequence
tataaattcg

SEQ ID NO: 11 - DNA, 35 bp, Artificial Sequence
  Oligonucleotide
caggctacggcacgtagagcatcaccatgatcctg

SEQ ID NO: 12 - DNA, 18 bp, Artificial Sequence
  oligonucleotide zip code
gctgccatcgatggtcag

SEQ ID NO: 13 - DNA, 18 bp, Artificial Sequence
  Oligonucleotide anti-zip code
ctgaccatcgatggcagc

SEQ ID NO: 14 - DNA, 18 bp, Artificial Sequence
  Oligonucleotide anti-zip code
ggactcgagcaccaatac

SEQ ID NO: 15 - DNA, 18 bp, Artificial Sequence
  Oligonucleotide zip code
gtattggtgctcgagtcc

SEQ ID NO: 16 - DNA, 15 bp, Artificial Sequence
  Oligonucleotide zip code
gctgccatcgatggt

SEQ ID NO: 17 - DNA, 15 bp, Artificial Sequence
  Oligonucleotide anti-zip code
accatcgatggcagc

SEQ ID NO: 18 - DNA, 13 bp, Artificial Sequence
  Oligonucleotide zip code
ttggtgctcgagt

SEQ ID NO: 19 - DNA, 31 bp, Artificial Sequence
  Oligo1 oligonucleotide
gtggtagttggagctggtggcgtaggcaaga

SEQ ID NO: 20 - DNA, 15 bp, Artificial Sequence
  Oligo2 oligonucleotide
agctccaactaccac

SEQ ID NO: 21 - DNA, 15 bp, Artificial Sequence
  Oligo4 oligonucleotide
gtggtagttggagct

SEQ ID NO: 22 - DNA, 15 bp, Artificial Sequence
  Oligo5 oligonucleotide
tcttgcctacgccac

SEQ ID NO: 23 - DNA, 15 bp, Artificial Sequence
  Oligo6 oligonucleotide
agatcccactagcac

SEQ ID NO: 24 - DNA, 18 bp, Artificial Sequence
  Oligo 26 oligonucleotide
cttcggcccagatatcgt

SEQ ID NO: 25 - DNA, 18 bp, Artificial Sequence
  Oligo 27 oligonucleotide
ctacagctacgatatctg

SEQ ID NO: 26 - DNA, 18 bp, Artificial Sequence
  Oligo 28 oligonucleotide
ctacagctgtgatatcat

SEQ ID NO: 27 - DNA, 20 bp, Artificial Sequence
  AT-rich oligonucleotide
tttttttttttttaattaaa

SEQ ID NO: 28 - DNA, 55 bp, Artificial Sequence
  Oligo 201 oligonucleotide
caggctacggcacgtagagcatcaccatgatcctgccccccccccatatttaagc

SEQ ID NO: 29 - DNA, 55 bp, Artificial Sequence
  Oligo 202 oligonucleotide
gcttaaatatcccccccccccaggctacggcacgtagagcatcaccatgatcctg

SEQ ID NO: 30 - DNA, 57 bp, Artificial Sequence
  Oligo 203 oligonucleotide
gtgggaatggtgcccccccccccaggctacggcacgtagagcatcaccatgatcctg

SEQ ID NO: 31 - DNA, 30 bp, Artificial Sequence
  Oligo 204 oligonucleotide
gtggtagttggagtcgtggcgtaggcaaga

SEQ ID NO: 32 - DNA, 34 bp, Artificial Sequence
  Oligo 205 oligonucleotide
gtggtagttggagtcacacgtggcgtaggcaaga

SEQ ID NO: 33 - DNA, 40 bp, Artificial Sequence
  Oligo 206 oligonucleotide
gtggtagttggagctcacaccacacgtggcgtaggcaaga

SEQ ID NO: 34 - DNA, 46 bp, Artificial Sequence
  Oligo 207 oligonucleotide
gtggtagttggagtcacacacaccacacacagtggcgtaggcaaga

SEQ ID NO: 35 - DNA, 68 bp, Artificial Sequence
  Oligo 208 oligonucleotide
gtggtagttggagctcacaccacaccaaccacaccacaccacacacaccacacgtggcgtaggcaaga

SEQ ID NO: 36 - DNA, 20 bp, Artificial Sequence
  Oligo 209 oligonucleotide
gtgtggtgtggtgtggtgtg

SEQ ID NO: 37 - DNA, 30 bp, Artificial Sequence
  Oligo 210 oligonucleotide
gtggcgtaggcaagagtggtagttggagct

SEQ ID NO: 38 - DNA, 12 bp, Artificial Sequence
  Oligo 211 oligonucleotide
gtgggaatggtg

SEQ ID NO: 39 - DNA, 15 bp, Artificial Sequence
  Oligo 212 oligonucleotide
agatcccactagcac

SEQ ID NO: 40 - DNA, 15 bp, Artificial Sequence
  Oligo 213 oligonucleotide
agctccaactaccac

SEQ ID NO: 41 - DNA, 15 bp, Artificial Sequence
  Oligo 214 oligonucleotide
tcttgcctacgccac

SEQ ID NO: 42 - DNA, 35 bp, Artificial Sequence; identical to SEQ ID NO: 11
  Oligo 215 oligonucleotide

SEQ ID NO: 43 - DNA, 28 bp, Artificial Sequence
  Oligo 106 oligonucleotide
  misc_feature (18)..(19): C18 PEG
ggactcgagcaccaatactataaattcg

SEQ ID NO: 44 - DNA, 28 bp, Artificial Sequence
  Oligo 109 oligonucleotide
  misc_feature (10)..(11): C18 PEG
cgaatttatactgaccatcgatggcagc

SEQ ID NO: 45 - DNA, 53 bp, Artificial Sequence
  Oligo 112 oligonucleotide
  misc_feature (35)..(36): C18 PEG
caggctacggcacgtagagcatcaccatgatcctggctgccatcgatggtcag

SEQ ID NO: 46 - DNA, 53 bp, Artificial Sequence
  Oligo 113 oligonucleotide
  misc_feature (18)..(19): C18 PEG
gtattggtgctcgagtcccaggctacggcacgtagagcatcaccatgatcctg

SEQ ID NO: 47 - DNA, 53 bp, Artificial Sequence; identical to SEQ ID NO: 46
  Oligo 119 oligonucleotide

SEQ ID NO: 48 - DNA, 63 bp, Artificial Sequence
  Oligo 120 oligonucleotide
gtattggtgctcgagtcccccccccccccaggctacggcacgtagagcatcaccatgatcctg

SEQ ID NO: 49 - DNA, 73 bp, Artificial Sequence
  Oligo 121 oligonucleotide
gtattggtgctcgagtcccccccccccccccccccccccaggctacggcacgtagagcatcaccatgatcctg

SEQ ID NO: 50 - DNA, 53 bp, Artificial Sequence; identical to SEQ ID NO: 45
  Oligo 122 oligonucleotide

SEQ ID NO: 51 - DNA, 63 bp, Artificial Sequence
  Oligo 123 oligonucleotide
caggctacggcacgtagagcatcaccatgatcctgccccccccccgctgccatcgatggtcag

SEQ ID NO: 52 - DNA, 73 bp, Artificial Sequence
  Oligo 124 oligonucleotide
caggctacggcacgtagagcatcaccatgatcctgccccccccccccccccccccgctgccatcgatggtcag

SEQ ID NO: 53 - DNA, 50 bp, Artificial Sequence
  Oligo 127 oligonucleotide
caggctacggcacgtagagcatcaccatgatcctggctgccatcgatggt

SEQ ID NO: 54 - DNA, 47 bp, Artificial Sequence
  Oligo 128 oligonucleotide
caggctacggcacgtagagcatcaccatgatcctggctgccatcgat

SEQ ID NO: 55 - DNA, 50 bp, Artificial Sequence
  Oligo 129 oligonucleotide
ttggtgctcgagtcccaggctacggcacgtagagcatcaccatgatcctg

SEQ ID NO: 56 - DNA, 47 bp, Artificial Sequence
  Oligo 130 oligonucleotide
gtgctcgagtcccaggctacggcacgtagagcatcaccatgatcctg

SEQ ID NO: 57 - DNA, 18 bp, Artificial Sequence
  Oligonucleotide zip code
gctgaggtacgatgctga

SEQ ID NO: 58 - DNA, 18 bp, Artificial Sequence
  Oligonucleotide anti-zip code
tcagcatcgtacctcagc

SEQ ID NO: 59 - DNA, 35 bp, Artificial Sequence
  Oligonucleotide
ttggtgctcgagtcccccccccccccccccccccc

SEQ ID NO: 60 - DNA, 38 bp, Artificial Sequence
  Oligonucleotide
ccccccccccccccccccccgctgaggtacgatgctga

SEQ ID NO: 61 - DNA, 15 bp, Artificial Sequence
  Oligo EDC1 oligonucleotide
gtggtagttggagct

SEQ ID NO: 62 - DNA, 15 bp, Artificial Sequence
  Oligo EDC2 oligonucleotide
agctccaactaccac

SEQ ID NO: 63 - DNA, 15 bp, Artificial Sequence
  Oligo EDC3 oligonucleotide
agatcccactagcac

SEQ ID NO: 64 - DNA, 35 bp, Artificial Sequence; identical to SEQ ID NO: 11
  Aptamer oligonucleotide

SEQ ID NO: 65 - DNA, 35 bp, Artificial Sequence; identical to SEQ ID NO: 11
  Aptamer oligonucleotide